(19) United States
(12) Patent Application Publication
(10) Pub. No.: US 2024/0400949 A1
(43) Pub. Date: Dec. 5, 2024

(54) COMPOSITION, COMPOUND, RESIN, SUBSTRATE TREATMENT METHOD, AND MANUFACTURING METHOD FOR SEMICONDUCTOR DEVICE

(71) Applicant: FUJIFILM Corporation, Tokyo (JP)

(72) Inventors: Naoya SHIMOJU, Haibara-gun (JP); Yasuo SUGISHIMA, Haibara-gun (JP); Tomonori TAKAHASHI, Haibara-gun (JP)

(73) Assignee: FUJIFILM Corporation, Tokyo (JP)

(21) Appl. No.: 18/800,584

(22) Filed: Aug. 12, 2024

Related U.S. Application Data
(63) Continuation of application No. PCT/JP2023/003361, filed on Feb. 2, 2023.

(30) Foreign Application Priority Data
Feb. 18, 2022 (JP) 2022-023833
Jun. 17, 2022 (JP) 2022-098218

(51) Int. Cl. C11D 7/32; C07C 211/27; C07C 217/60; C08F 12/22; C08F 12/28; C08F 20/06; C08F 20/28; C08F 20/56; C08F 22/02; C08F 22/38; C08F 26/06; C08F 28/02; C11D 7/34; H01L 21/02

(52) U.S. Cl. CPC C11D 7/3245; C07C 211/27; C07C 217/60; C08F 12/22; C08F 12/28; C08F 20/06; C08F 20/28; C08F 20/56; C08F 22/02; C08F 22/38; C08F 26/06; C08F 28/02; C11D 7/3209; C11D 7/3281; C11D 7/34; H01L 21/0206

(57) ABSTRACT

The present invention provides a composition for a semiconductor device, where the composition is such that the removability of residues is excellent and the dissolution of tungsten is further suppressed. The composition for a semiconductor device contains a resin having a repeating unit A derived from a polymerizable compound containing a nitrogen atom and water, where a ClogP of the polymerizable compound is 0.5 or more and a solubility of the resin in water at 25° C. is 0.01% by mass or more.

### CROSS-REFERENCE TO RELATED APPLICATIONS

This application is a Continuation of PCT International Application No. PCT/JP2023/003361 filed on Feb. 2, 2023, which claims priority under 35 U.S.C. § 119(a) to Japanese Patent Application No. 2022-023833 filed on Feb. 18, 2022 and Japanese Patent Application No. 2022-098218 filed on Jun. 17, 2022. The above applications are hereby expressly incorporated by reference, in their entirety, into the present application.

### BACKGROUND OF THE INVENTION

### 1. Field of the Invention

The present invention relates to a composition, a compound, a resin, a substrate treatment method, and a manufacturing method for a semiconductor device.

### 2. Description of the Related Art

With the progress of miniaturization of semiconductor products, there is an increasing demand for carrying out a step of removing unnecessary metal-containing substances on a substrate with high efficiency and accuracy in a semiconductor product manufacturing process.

Semiconductor devices are manufactured, for example, by disposing a laminate having a metal layer serving as a wiring line material, an etching stop film, and an insulating film on a substrate, forming a resist film on this laminate, and carrying out a photolithography step and a dry etching step.

In the photolithography step, a method of carrying out etching or a method of removing a foreign substance that has adhered to the surface of a substrate by using a composition that dissolves a metal-containing substance is widely known.

For example, in the photolithography step, a metal layer and/or an insulating film on a substrate may be etched by a dry etching treatment using a resist film as a mask. In this case, residues derived from the metal layer and/or the insulating film and the like may adhere to the substrate, the metal layer, and/or the insulating film. In order to remove the adhered residues, washing using a composition is often carried out.

In addition, the resist film that is used as a mask at the time of etching is then removed from the laminate by a dry-type method (dry ashing) with ashing (incineration) or a wet-type method. The residues derived from the resist film or the like may adhere to the laminate from which the resist has been removed by using the dry ashing method.

Further, nowadays, a resist film (a so-called metal hard mask) based on a metal material such as TiN or AlOx has been studied as the resist film in order to realize further miniaturization of semiconductor devices. In a case where the metal hard mask is used as the resist film, a dry etching step (for example, a plasma etching treatment) is carried out using the metal hard mask as a mask and a step of forming holes based on a pattern shape of the metal hard mask and exposing a surface of a metal film serving as a wiring line film are usually carried out.

Etching residues and/or ashing residues are deposited on the substrate that has undergone the dry etching step or the dry ashing step. In a case where a metal hard mask is used as the resist film, the residue component includes, for example, a large amount of a metal component such as a titanium-based metal, and in a case where a photoresist film is used, the residue component includes a large amount of an organic component.

In order to prevent these adhered residues from interfering with the subsequent step, a washing treatment of removing the residues is often carried out using a composition. In addition, examples of the wet-type method of removing the resist film include an aspect in which the resist film is removed using a composition.

As described above, the composition for a semiconductor device is used for a treatment such as the removal of metal-containing substances (etching residues and ashing residues) on a substrate and/or a resist film in the semiconductor device manufacturing step.

For example, JP2012-094852A describes a washing solution that is used in a washing step for a substrate for a semiconductor device, where the washing solution is a washing solution for a substrate for a semiconductor device, containing at least one polymer coagulating agent selected from an organic acid, a sulfonic acid type anionic surfactant, polyvinylpyrrolidone, and a polyethylene oxide-polypropylene oxide block copolymer, and water.

### SUMMARY OF THE INVENTION

As a result of studying a composition for a semiconductor device with reference to JP2012-094852A, the inventors of the present invention found that there is room for further improvement in the performance of suppressing the dissolution of tungsten in a case where the composition is applied to a laminate having a tungsten-containing layer.

Therefore, an object of the present invention is to provide a composition for a semiconductor device, where the composition is such that the removability of residues is excellent and the dissolution of tungsten is further suppressed.

In addition, another object of the present invention is to provide a substrate treatment method using the above composition and a manufacturing method for a semiconductor device.

Further, another object of the present invention is to provide a compound and a resin.

As a result of diligent studies to achieve the objects, the inventors of the present invention found that the above objects can be achieved by a composition containing a specific resin, whereby the present invention was completed.

That is, the inventors of the present invention found that the above-described objects can be achieved by the following configurations.

[1] A composition for a semiconductor device, comprising:
a resin having a repeating unit A derived from a polymerizable compound containing a nitrogen atom; and
water,
in which a ClogP of the polymerizable compound is 0.5 or more, and
a solubility of the resin in water at 25° C. is 0.01% by mass or more.

[2] The composition according to [1], in which the polymerizable compound includes at least one selected from the group consisting of compounds represented by Formulae (1) to (3) described later.

[3] The composition according to [1] or [2], in which the polymerizable compound has at least one selected from the group consisting of a primary amino group, a secondary amino group, a tertiary amino group, and salts thereof.

[4] The composition according to any one of [1] to [3], in which the polymerizable compound has a primary amino group or a salt thereof.

[5] The composition according to any one of [1] to [4], in which the polymerizable compound has an aromatic ring.

[6] The composition according to any one of [1] to [5], in which the resin is a compound represented by Formula (1a) described later.

[7] The composition according to any one of [1] to [6], in which the resin further has a repeating unit B having an acid group.

[8] The composition according to [7], in which the acid group is a carboxy group.

[9] The composition according to [7] or [8], in which a ratio a/b of a molar number a of the repeating unit A to a molar number b of the repeating unit B is 10/90 to 80/20 where the repeating unit A and the repeating unit B are contained in the resin.

[10] The composition according to any one of [1] to [9], in which the resin has a weight-average molecular weight of 1,000 to 500,000.

[11] The composition according to any one of [1] to [10], further comprising a removing agent.

[12] The composition according to any one of [1] to [11], in which a content of the resin is 10 to 10,000 ppm by mass with respect to a total mass of the composition.

[13] The composition according to any one of [1] to [12], further comprising at least one selected from the group consisting of an oxidizing agent, a corrosion inhibitor, a surfactant, an antifoaming agent, and an organic solvent.

[14] The composition according to any one of [1] to [13], in which the composition is a composition for a substrate having a metal-containing which contains at least one of tungsten or a tungsten alloy.

[15] The composition according to any one of [1] to [14], in which the composition is used as a washing solution for removing etching residues or a washing solution for removing residues from a substrate after chemical mechanical polishing.

[16] A substrate treatment method comprising:
a step A of removing a metal-containing substance on a substrate by using the composition according to any one of [1] to [15].

[17] The substrate treatment method according to [16], further comprising a step C of subjecting the substrate obtained in the step A to a rinsing treatment by using a rinsing liquid.

[18] A manufacturing method for a semiconductor device, comprising:
a step of treating a substrate by using the composition according to any one of [1] to [15].

[19] A compound represented by Formula (1a) described later.

[20] A resin comprising:
a repeating unit A derived from the compound according to [19].

[21] A composition comprising:
the compound according to [19]; or
the resin according to [20].

[22] A compound represented by Formula (1b) described later.

According to the present invention, it is possible to provide a composition for a semiconductor device, where the composition is such that the removability of residues is excellent and the dissolution of tungsten is further suppressed.

In addition, the present invention can provide a substrate treatment method using the above composition and a manufacturing method for a semiconductor device.

In addition, the present invention can provide a compound and a resin.

### BRIEF DESCRIPTION OF THE DRAWINGS

FIG. 1 is a schematic cross-sectional view illustrating an example of a laminate which is an object to be treated in a substrate treatment method.

### DESCRIPTION OF THE PREFERRED EMBODIMENTS

Hereinafter, the present invention will be described in detail.

Descriptions of the configuration requirements which will be described later are made based on representative embodiments of the present invention in some cases, but it should not be construed that the present invention is limited to such embodiments.

In the present specification, the numerical value range indicated by using “to” means a range including the numerical values before and after “to” as the lower limit value and the upper limit value, respectively.

In the present specification, in a case where two or more kinds of certain components are present, the “content” of the component means a total content of the two or more kinds of the components.

The compound described in the present specification may include a structural isomer, an optical isomer, and an isotope unless otherwise specified. In addition, one kind of structural isomer, optical isomer, and isotope alone, or two or more kinds thereof may be included.

In the present specification, the “total solid content” means the total content of all components contained in the composition other than a solvent such as water or an organic solvent.

In the present specification, the term “preparing” includes not only preparing a predetermined substance by a treatment such as synthesis or blending of raw materials, but also procuring a predetermined substance by purchasing or the like.

In the present specification, “psi” means pound-force per square inch, and 1 psi means 6,894.76 Pa.

In the present specification, “ppm” means “parts-per-million (10⁻⁶)”, “ppb” means “parts-per-billion (10⁻⁹)”, and “ppt” means “parts-per-trillion (10⁻¹²)”.

In the present specification, 1 angstrom (Å) corresponds to 0.1 nm.

In the present specification, unless otherwise specified, the weight-average molecular weight (Mw) and the number average molecular weight (Mn) are values obtained by using TSKgel GMHxL, TSKgel G4000HxL, or TSKgel G2000HxL (all of which are manufactured by Tosoh Corporation) as a column, using tetrahydrofuran as an eluent, using a differential refractometer as a detector, using polystyrene as a standard substance, and carrying out conversion using the polystyrene as a standard substance, which has been subjected to measurement with a gel permeation chromatography (GPC) analysis apparatus. In the present specification, unless otherwise specified, a molecular weight of a compound having a molecular weight distribution is a weight-average molecular weight.

In the present specification, the term “radiation” means, for example, a bright line spectrum of a mercury lamp, far ultraviolet rays typified by an excimer laser, extreme ultraviolet rays (EUV light), X-rays, or electron beams. In the present specification, light means actinic rays or radiation. Unless otherwise specified, the “exposure” in the present specification includes not only exposure with a bright line spectrum of a mercury lamp, far ultraviolet rays represented by an excimer laser, X-rays, or EUV light, but also the exposure includes drawing with particle beams such as electron beams and ion beams.

### [Composition]

A composition according to the embodiment of the present invention (hereinafter, also referred to as the “present composition”) is a composition for a semiconductor device, which includes a resin having a repeating unit A derived from a polymerizable compound having a nitrogen atom, and water. In addition, the solubility of the resin in water at 25° C. is 0.01% by mass or more, and the ClogP of the polymerizable compound is 0.5 or more. Hereinafter, the resin having the above-described specific repeating unit A and having a solubility of 0.01% by mass or more in water at 25° C. is also referred to as a “specific resin”.

The inventors of the present invention have found that the present composition including the above-described specific resin together with water makes it possible to obtain such an effect (hereinafter, also described as “the effect of the present invention”) that the removability of residues is excellent and the dissolution of tungsten is further suppressed, whereby the present invention was completed.

Although a detailed mechanism by which the effect of the present invention is obtained with the present composition is unknown, the inventors of the present invention presume that in a case where the present composition is used for the treatment of a laminate having a tungsten-containing layer, a suitable protective film composed of the specific resin is formed on the surface of the tungsten-containing layer, and thus the elution of tungsten from the tungsten-containing layer into the composition can be further suppressed.

Hereinafter, each component contained in the present composition will be described in detail.

### [Specific Resin]

The specific resin is a resin which has a repeating unit A derived from a polymerizable compound having a nitrogen atom and having a ClogP of 0.5 or more and has a solubility of 0.01% by mass or more in water at 25° C.

The polymerizable compound (hereinafter, also referred to as a “polymerizable compound A”) from which the repeating unit A is derived is not particularly limited as long as it is a compound that has a nitrogen atom and a polymerizable group and has a ClogP of 0.5 or more.

Examples of the polymerizable group contained in the polymerizable compound A include a radically polymerizable group such as an ethylenically unsaturated group. Examples of the radically polymerizable group include a vinyl group, an allyl group, a (meth)acryloyl group, and a (meth)acrylamide group.

The number of polymerizable groups contained in the polymerizable compound A may be only one or may be two or more; however, it is preferably one.

The ClogP of the polymerizable compound A is 0.5 or more, and it is preferably 0.7 or more and more preferably 1.0 or more from the viewpoint that the effect of the present invention is more excellent. The upper limit of ClogP is not particularly limited; however, it is preferably 4.0 or less from the viewpoint that the solubility of the specific resin in water is more excellent.

In the present specification, the term “ClogP” is a value determined by calculating the common logarithm logP of a partition coefficient P of 1-octanol to water. The ClogP value can be calculated using a publicly known method and publicly known software. However, unless otherwise specified, in the present specification, ClogP means a ClogP value calculated using a ClogP program incorporated in “Chem Bio Draw Ultra Version 20” (manufactured by Cambridge Soft Corporation).

It is noted that a ClogP value of a polymerizable compound from which a repeating unit is derived is determined by measuring the structure of the repeating unit contained in the resin by a method described later, estimating the structure of the polymerizable compound from which the repeating unit is derived, from the obtained structure, and calculating the ClogP value using the above-described software based on the estimated structure of the polymerizable compound, where the estimated structure includes a polymerizable group.

The solubility of the specific resin in water at 25° C. is 0.01% by mass or more. That is, the concentration of the saturated aqueous solution of the specific resin at 25° C. is 0.01% by mass or more.

From the viewpoint that the suppression of precipitation from the composition is more excellent, the solubility of the specific resin in water at 25° C. is preferably 0.01% by mass or more and more preferably 0.03% by mass or more. The upper limit thereof is not particularly limited, and it is, for example, 30% by mass or less.

The solubility of the specific resin in water at 25° C. can be measured by the integrating sphere type photoelectric photometry according to JIS K 0101 (2017) using a turbidity meter (for example, “PT-200” manufactured by Mitsubishi Chemical Analytech Co., Ltd.).

### <Polymerizable Compound A>

The polymerizable compound A preferably has, as a functional group containing a nitrogen atom, at least one group (hereinafter, also referred to as a “specific amino group”) selected from the group consisting of a primary amino group, a secondary amino group, a tertiary amino group, and salts these groups, and a quaternary ammonium cationic group.

The above-described salts of the primary amino group, the secondary amino group, and the tertiary amino group are salts formed of the respective amino groups and an acidic compound. In addition, the above-described secondary amino group, tertiary amino group, and quaternary ammonium cationic group may be linked to another group contained in the polymerizable compound A to form a nitrogen-containing heterocyclic ring having at least one specific amino group. In addition, the quaternary ammonium cationic group may form a salt with a counter ion corresponding to the acidic compound.

The acidic compound may be any one of an inorganic acid or an organic acid.

Examples of the inorganic acid include hydrochloric acid, sulfuric acid, phosphoric acid, and nitric acid. Examples of the organic acid include acetic acid, propionic acid, methanesulfonic acid, ethanesulfonic acid, allylglycine, maleic acid, citraconic acid, fumaric acid, and itaconic acid.

The acid in a case where the specific amino group forms a salt is preferably hydrochloric acid, acetic acid, propionic acid, methanesulfonic acid, or ethanesulfonic acid, more preferably hydrochloric acid, acetic acid, methanesulfonic acid, or ethanesulfonic acid, and still more preferably hydrochloric acid, acetic acid, or ethanesulfonic acid.

The number of the specific amino groups contained in the polymerizable compound A is not particularly limited; however, it is preferably an integer of 1 to 4, more preferably 1 to 3, still more preferably 1 or 2, and particularly preferably 1.

In a case where the polymerizable compound A has at least one selected from the group consisting of a secondary amino group (—NHR), a tertiary amino group (—NR₂), and a quaternary ammonium cationic group (—N⁺R₃), one, two, or three substituents R which are contained on the nitrogen atom of the secondary amino group, the tertiary amino group, or the quaternary ammonium cationic group are not particularly limited; however, it is preferably a substituted or unsubstituted hydrocarbon group, more preferably a substituted or unsubstituted linear or branched alkyl group having 1 to 8 carbon atoms, and still more preferably a substituted or unsubstituted linear or branched alkyl group having 1 to 4 carbon atoms. The substituent is preferably a carboxy group. In addition, in the tertiary amino group or the quaternary ammonium cationic group, a plurality of R's may be the same or different from each other.

In addition, in a case where the polymerizable compound A has a nitrogen-containing heterocyclic ring having at least one specific amino group, the number of nitrogen atoms contained in the nitrogen-containing heterocyclic ring is not particularly limited; however, it is preferably an integer of 1 to 4, more preferably 1 to 3, and still more preferably 1 or 2. The number of ring members in the nitrogen-containing heterocyclic ring is preferably an integer of 5 to 7 and more preferably 5 or 6. In addition, the nitrogen-containing heterocyclic ring may be any of an aromatic ring or a non-aromatic ring; however, it is preferably an aromatic ring.

From the viewpoint that the effect of the present invention is more excellent, the specific amino group contained in the polymerizable compound A is preferably a group selected from the group consisting of a primary amino group, a secondary amino group, a tertiary amino group, and salts thereof, and more preferably a primary amino group or a salt thereof. In addition, it is also preferable that the polymerizable compound A has at least one primary amino group or a salt thereof and further has one group selected from the group consisting of a primary amino group, a secondary amino group, a tertiary amino group, and salts thereof.

From the viewpoint that the effect of the present invention is more excellent and the viewpoint that the effect of suppressing the dissolution of the titanium-containing film is more excellent, the polymerizable compound A preferably has an aromatic ring. The aromatic ring may be an aromatic hydrocarbon ring or may be a heteroaromatic ring containing a heteroatom. The polymerizable compound A may have, as the above-described heteroaromatic ring, a nitrogen-containing heteroaromatic ring having at least one secondary amino group or one tertiary amino group.

Examples of the aromatic hydrocarbon ring include a benzene ring and a naphthalene ring, and a benzene ring is preferable.

Examples of the heteroaromatic ring include an imidazole ring, a pyrazole ring, a thiazole ring, a triazole ring, a tetrazole ring, a pyridine ring, a pyrazine ring, a pyrimidine ring, and a triazine ring, and among these rings, an imidazole ring, a pyridine ring, or a triazine ring is preferable.

Examples of the polymerizable compound A include a compound represented by Formula (a).

Q-L-(X)ₙ (a)

In Formula (a), Q represents an ethylenically unsaturated group which may have a substituent.

L represents a direct bond or an (n+1)-valent linking group.

X represents a specific amino group or a nitrogen-containing heterocyclic ring having at least one specific amino group.

n represents an integer of 1 to 5. However, in a case where L represents a direct bond, n represents 1.

In a case where n is an integer of 2 to 5, a plurality of X's may be the same or different from each other.

m represents 1 or 2.

The ethylenically unsaturated group represented by Q and the linking group represented by L may be linked to each other to form a ring.

The ethylenically unsaturated group represented by Q may have a substituent. The number of substituents is, for example, an integer of 1 to 3, and it is preferably 1. The ethylenically unsaturated group represented by Q preferably has no substituent or has only one substituent.

Examples of the substituent contained in the ethylenically unsaturated group represented by Q include an alkyl group having 1 to 4 carbon atoms. The ethylenically unsaturated group represented by Q may further have a group represented by {L-(X)}ₙ. In that case, the definitions and preferred aspects of L, X, and n in the group represented by {L-(X)}ₙ are the same as those described above.

The substituent contained in the ethylenically unsaturated group represented by Q is preferably an alkyl group having 1 to 4 carbon atoms, more preferably a methyl group or an ethyl group, and still more preferably a methyl group.

The linking group represented by L is not particularly limited as long as it is a group having a valence corresponding to the number of X's. However, examples thereof include an aliphatic hydrocarbon group, a group obtained by substituting at least one methylene group included in an aliphatic hydrocarbon group with a group selected from the group consisting of —O—, —CO—, —NH—, and —NR— (R represents an alkyl group), an aromatic hydrocarbon ring, a heteroaromatic ring, and a group obtained by combining these.

Among these, It is preferably a group obtained by eliminating hydrogen atoms of which the number corresponds to the number of X's for substitution, from a group selected from the group consisting of an aromatic hydrocarbon ring, a group obtained by combining an aromatic hydrocarbon ring and a linear or branched aliphatic hydrocarbon group, a heteroaromatic ring, a group obtained by combining an aliphatic hydrocarbon group and an ester bond (—COO—), and a group obtained by combining an aliphatic hydrocarbon group and an amide bond (—CONH—).

Suitable aspects of the above-described aromatic hydrocarbon ring and the above-described heteroaromatic ring are as described above.

The above-described aliphatic hydrocarbon group may be linear, branched, or cyclic; however, it is preferably linear or branched. The number of carbon atoms in the aliphatic hydrocarbon group is preferably 3 to 12, and more preferably 4 to 10.

In addition, the aliphatic hydrocarbon group which is combined with the aromatic hydrocarbon group is preferably an aliphatic hydrocarbon group having 1 to 4 carbon atoms, and more preferably a methylene group or an ethylene group.

A preferred aspect of the specific amino group represented by X and a preferred aspect of the nitrogen-containing heterocyclic ring having at least one specific amino group are as described above.

n preferably represents an integer of 1 to 3 and more preferably represents 1 or 2.

The combination of X and L is preferably a combination in which X (at least one X in a case where n is an integer of 2 or more) represents a specific amino group and L represents an (n+1)-valent linking group, or a combination in which X represents a nitrogen-containing heterocyclic ring having at least one specific amino group and L represents a direct bond.

The polymerizable compound A is preferably a compound represented by Formula (1), a compound represented by Formula (2), or a compound represented by Formula (3).

CH₂═CR¹-L¹-(X¹)ₙ₁ (1)

In Formula (1), R¹ represents a hydrogen atom or an alkyl group having 1 to 4 carbon atoms.

L¹ represents an (n1+1)-valent linking group.

X¹ represents a specific amino group.

n1 represents an integer of 1 to 5.

In a case where n1 is an integer of 2 to 5, a plurality of X's may be the same or different from each other.

In Formula (1), R¹ is preferably a hydrogen atom, a methyl group, or an ethyl group, and more preferably a hydrogen atom or a methyl group.

A preferred aspect of the (n1+1)-valent linking group represented by L¹ is the same as that of the linking group represented by L in Formula (a).

A preferred aspect of the specific amino group represented by X¹ is as described above.

n1 preferably represents an integer of 1 to 3 and more preferably represents 1 or 2.

CH₂=CR²—X² (2)

In Formula (2), R² represents a hydrogen atom or an alkyl group having 1 to 4 carbon atoms.

X² represents a nitrogen-containing heterocyclic ring having at least one specific amino group.

In Formula (2), R² is preferably a hydrogen atom, a methyl group, or an ethyl group, and more preferably a hydrogen atom or a methyl group.

The nitrogen-containing heterocyclic ring having at least one specific amino group, which is represented by X², is preferably a nitrogen-containing heteroaromatic ring having at least one secondary amino group or one tertiary amino group, and more preferably an imidazole ring, a pyridine ring, or a triazine ring.

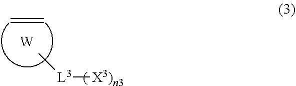

In Formula (3), a ring W represents a hydrocarbon ring or a heterocyclic ring, which has an ethylenically unsaturated group.

L³ represents a single bond or an (n3+1)-valent linking group.

X³ represents a specific amino group.

n3 represents an integer of 1 to 5.

In a case where n3 is an integer of 2 to 5, a plurality of X³'s may be the same or different from each other.

In Formula (3), the ring W is not particularly limited as long as it is a hydrocarbon ring or a heterocyclic ring, in which a part of the ring is an ethylenically unsaturated group. The number of ring members in the ring W is preferably an integer of 5 to 7 and more preferably 5 or 6. The ring W may further have a substituent. Examples of the substituent include an alkyl group having 1 to 4 carbon atoms and a hydroxy group. In addition, at least one of the methylene groups constituting the ring W may be replaced with a carbonyl group.

Examples of the ring W include a cyclopentene ring, a cyclohexene ring, a pyrroline ring, and a maleimide ring, and a maleimide ring is preferable.

L³ is preferably an aliphatic hydrocarbon group, an aromatic hydrocarbon ring, a heteroaromatic ring, or a group obtained combining an aromatic hydrocarbon ring or heteroaromatic ring and an aliphatic hydrocarbon group, and more preferably an aliphatic hydrocarbon group having 1 to 4 carbon atoms, a benzene ring, or a group obtained combining a benzene ring and an aliphatic hydrocarbon group having 1 to 4 carbon atoms.

A preferred aspect of the specific amino group represented by X3 is as described above.

n3 preferably represents an integer of 1 to 3 and more preferably represents 1 or 2.

The polymerizable compound A is more preferably a compound represented by Formula (1) or a compound represented by Formula (2), and still more preferably a compound represented by Formula (1).

Examples of the preferred aspect of the compound represented by Formula (1) include a compound represented by Formula (1a) (hereinafter, also referred to as a “compound 1a”).

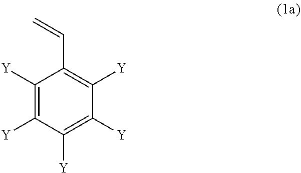

In the formula, Y's each independently represent a hydrogen atom or -L²-L³-NH₂. However, at least two Y's represent -L²-L³-NH₂.

L² represents a single bond or an oxygen atom.

L³ represents an aliphatic hydrocarbon group having 1 to 5 carbon atoms.

It is noted that the compound 1a also includes a compound in which the NH₂ contained in Y forms a salt with an acidic compound. The acidic compound that forms a salt with NH₂ contained in Y is the same as the acidic compound that forms a salt with an amino group such as a primary amino group which is contained in the polymerizable compound A, including the preferred aspect of the acidic compound.

In the compound 1a, it is preferable that two or three Y's each independently represent -L²-L³-NH₂, and it is more preferable that two Y's each independently represent -L²-L³-NH₂. That is, in Formula (1a), it is preferable that two or three Y's represent hydrogen atoms, and it is more preferable that three Y's represent hydrogen atoms.

L² is preferably a single bond.

L³ is preferably a linear or branched alkylene group having 1 to 3 carbon atoms, more preferably a methylene group or an ethylene group, and still more preferably a methylene group.

The compound 1a can be synthesized according to a publicly known synthesis method for an amine compound.

For example, the compound 1a can be synthesized by condensing a compound represented by Formula (1b) (hereinafter, also referred to as a “compound 1b”) and phthalimide according to a Gabriel amine synthesis reaction for synthesizing a primary amine using phthalimide, under the conditions of the Mitsunobu reaction using an azocarboxylic acid ester and a phosphine compound, and then carrying out amination with a deprotection reaction using a hydrazine compound.

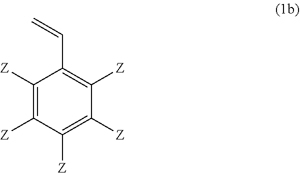

In the formula, Z's each independently represent a hydrogen atom or -L²-L³-OH. However, at least two Z's represent -L²-L³-OH.

A preferred aspect of the compound 1b that is used for synthesizing the compound 1a is the same as the preferred aspect of the compound 1a, except that the primary amino group (—NH₂) is a hydroxyl group (—OH).

The compound 1b can be synthesized according to a publicly known synthesis method for reducing a carboxylic acid ester group to synthesize a primary alcohol. More specifically, the compound 1b can be synthesized by, for example, allowing hydrogenated aluminum lithium to act on a styrene derivative having a carboxylic acid ester group corresponding to Z described above, thereby reducing the carboxylic acid ester group. The compound 1b can also be synthesized by carrying out the above-described reduction reaction using sodium borohydride or the like instead of hydrogenated aluminum lithium.

Examples of the more detailed synthesis method for the compound 1b include a method described in Examples described later.

In the synthesis of the compound 1a, a nucleophilic agent having an amino group having an acidic proton can also be used instead of phthalimide.

Examples of the azocarboxylic acid ester that is used in the condensation reaction between the compound 1a and phthalimide include publicly known Mitsunobu reagents such as bis(2-methoxyethyl) azodicarboxylate, diisopropyl azodicarboxylate, and diethyl azodicarboxylate. From the viewpoint of simple purification, bis(2-methoxyethyl) azodicarboxylate is preferable.

Examples of the phosphine compound that is used in the condensation reaction between the compound 1a and phthalimide include triphenylphosphine.

In the condensation reaction between the compound 1a and phthalimide, it is preferable to use an aprotic polar solvent from the viewpoint of improving the yield. Examples of the aprotic polar solvent include tetrahydrofuran (THF), dimethylformamide (DMF), and dioxane.

Examples of the hydrazine compound that is used in the above-described deprotection reaction of the condensate include hydrazine and a derivative thereof, and hydrazine is preferable.

Examples of the solvent that is used in the above-described deprotection reaction of the condensate include alcohol and water. A monohydric alcohol having 1 to 5 carbon atoms is preferable, and ethanol is more preferable.

Examples of the more detailed synthesis method for the compound 1a include a method described in Examples described later.

Hereinafter, specific examples of the polymerizable compound A will be shown below together with the ClogP value of each compound.

in the formulae, AcO⁻ represents an acetate (CH₃—C(═O)—O⁻), and MsO⁻ represents a methanesulfonate (CH₃—SO₂—O⁻).

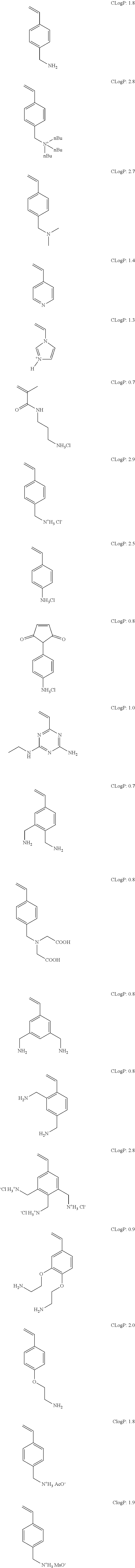

The specific resin may have only one kind of the repeating unit A derived from the polymerizable compound A or may have two or more kinds thereof. In a case where the specific resin has two or more kinds of repeating units A, it is preferable to have at least one repeating unit represented by Formula (1) or Formula (2).

From the viewpoint that the effect of the present invention is more excellent, the content of the repeating unit A in the specific resin is preferably 1% by mole or more, more preferably 5% by mole or more, still more preferably 10% by mole or more, and particularly preferably 25% by mole or more with respect to all repeating units in the specific resin.

The upper limit of the content of the repeating unit A is not particularly limited; however, it is preferably 99% by mole or less, more preferably 95% by mole or less, still more preferably 80% by mole or less, and particularly preferably 70% by mole or less with respect to all repeating units in the specific resin.

It is noted that the structure and the compositional ratio (molar fraction) between the respective repeating units contained in the specific resin can be measured by ¹³C-NMR.

The specific resin may have another repeating unit which is different from the repeating unit A.

Here, “the repeating unit different from the repeating unit A” means a repeating unit derived from any of a polymerizable compound that does not contain a nitrogen atom but has a ClogP of 0.5 or more, a polymerizable compound that contains a nitrogen atom and has a ClogP of less than 0.5, and a polymerizable compound that does not contain a nitrogen atom but has a ClogP of less than 0.5.

### <Repeating Unit B>

It is preferable that the specific resin further has a repeating unit B having an acid group, as a repeating unit different from the repeating unit A.

In a case where the specific resin has the repeating unit B, the solubility of the composition in a post-treatment liquid such as a rinsing liquid which is carried out after the treatment is carried out using the composition is improved, which makes it possible to further reduce the amount of the residue derived from the composition on a surface of a substrate such as a metal film after the post-treatment is carried out using the post-treatment liquid.

Examples of the acid group contained in the repeating unit B include a carboxy group, a phosphonic acid group, a sulfo group, and a phenolic hydroxyl group. A carboxy group or a sulfo group is preferable, and a carboxy group is more preferable from the viewpoint that the solubility of the composition in the post-treatment liquid is more excellent.

The number of acid groups contained in the repeating unit B is not particularly limited; however, it is preferably 1 to 4, more preferably 1 to 3, and still more preferably 1 or 2.

Examples of the repeating unit B include a repeating unit represented by Formula (b).

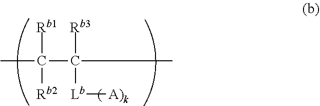

in Formula (b), R^{b1}, R^{b2}, and R^{b3} each independently represent a hydrogen atom, an alkyl group having 1 to 4 carbon atoms, or an acid group.

L^{b} represents a single bond or a (k+1)-valent linking group.

A represents an acid group.

k represents an integer of 1 to 4.

In a case where a plurality of acid groups are present in Formula (b), the plurality of acid groups may be the same or different from each other.

R^{b1}, R^{b2}, and R^{b3} are preferably a hydrogen atom, a methyl group, an ethyl group, or a carboxy group, and more preferably a hydrogen atom, a methyl group, or a carboxy group.

Among those, it is preferable that one of R^{b1}, R^{b2}, and R^{b3} represents a hydrogen atom, a methyl group, or a carboxy group, and both the remaining two thereof represent a hydrogen atom.

The linking group represented by L^{b} is not particularly limited as long as it is a group having a valence corresponding to the number of A's. However, examples thereof include an aliphatic hydrocarbon group, a group obtained by substituting at least one methylene group included in an aliphatic hydrocarbon group with —O—, or —CO—, an aromatic hydrocarbon ring, a heteroaromatic ring, and a group obtained by combining these.

Among these, it is preferably a group obtained by eliminating hydrogen atoms of which the number corresponds to the number of A's for substitution, from a group selected from the group consisting of an alkyl group having 1 to 4 carbon atoms, a group obtained by substituting at least one methylene group included in an aliphatic hydrocarbon group having 2 to 10 carbon atoms with —O—, or —CO—, and a phenyl group.

L^{b} in Formula (b) is preferably a single bond or a linking group in the above-described preferred aspect, and more preferably a single bond, a methylene group, or a phenylene group.

A preferred aspect of the acid group represented by A is as described above.

k is preferably an integer of 1 to 3 and more preferably 1 or 2.

In a case where the specific resin has the repeating unit B, only one kind of the repeating unit B may be used, or two or more kinds thereof may be combined. In a case where the specific resin has two or more kinds of repeating units B, it is preferable to have at least one repeating unit represented by Formula (b).

From the viewpoint that the solubility of the composition in the post-treatment liquid is more excellent, the content of the repeating unit B is preferably 1% by mole or more, more preferably 5% by mole or more, still more preferably 20% by mole or more, and particularly preferably 30% by mole or more with respect to all repeating units in the specific resin. The upper limit thereof is not particularly limited; however, it is preferably 99% by mole or less, more preferably 95% by mole or less, still more preferably 90% by mole or less, and particularly preferably 75% by mole or less with respect to all repeating units in the specific resin.

The ratio of the repeating unit A to the repeating unit B in the specific resin is not particularly limited. However, from the viewpoint that the effect of the present invention is more excellent, a ratio a/b of a molar number a of the repeating unit A to a molar number b of the repeating unit B is preferably 1/99 or more, more preferably 5/95 or more, still more preferably 10/90 or more, and particularly preferably 25/75 or more.

The lower limit of the ratio a/b is not particularly limited. However, from the viewpoint that the solubility of the composition in the post-treatment liquid is more excellent, the ratio a/b is preferably 99/1 or less, more preferably 95/5 or less, still more preferably 80/20 or less, and particularly preferably 70/30 or less.

The polymerizable compound from which the repeating unit B is derived (hereinafter, also referred to as a “polymerizable compound B”) is not particularly limited as long as it is a compound that has an acid group and a polymerizable group and forms the specific resin having the repeating unit A and the repeating unit B by copolymerization with the above-described polymerizable compound A.

The acid group contained in the polymerizable compound B is as described above, including the preferred aspect thereof.

In addition, the polymerizable group contained in the polymerizable compound B is the same as the polymerizable group contained in the polymerizable compound A, including the preferred aspect thereof.

Examples of the polymerizable compound B include a compound from which the repeating unit represented by Formula (b) is derived, and the preferred aspect thereof is the same as that of the repeating unit represented by Formula (b).

The specific resin may have a repeating unit different from both the repeating unit A and the repeating unit B. In the specific resin, the content of the repeating unit different from both the repeating unit A and the repeating unit B is, for example, 20% by mole or less, preferably 0% to 10% by mole, and more preferably 0% to 5% by mole with respect to all repeating units in the specific resin.

It is preferable that the specific resin does not have a repeating unit different from both the repeating unit A and the repeating unit B.

Specific examples of the specific resin include resins represented by Formulae (E-1) to (E-24). In Formulae (E-1) to (E-6), (E-8) to (E-19), and (E-22) to (E-24), the repeating unit described on the left side of the paper surface is the repeating unit A, and the repeating unit described on the right side of the paper surface is the repeating unit B. In addition, in the resins (E-20) and (E-21), among the three repeating units, the repeating unit on the left side of the paper surface is the repeating unit A, and the two repeating units in the center and on the right side of the paper surface are the repeating unit B.

In the formulae, AcO⁻ represents an acetate (CH₃—C(═O)—O—), and MsO⁻ (CH₃—SO₂—O⁻) represents a methanesulfonate.

It is noted that in the resins represented by Formulae (E-1) to (E-6) and (E-8) to (E-24), the bonding modes of a plurality of repeating units are not particularly limited. For example, the plurality of repeating units may be bonded randomly (so-called the random copolymer), may be alternately bonded (so-called the alternating copolymer), or may be bonded in a block shape (so-called the block copolymer).

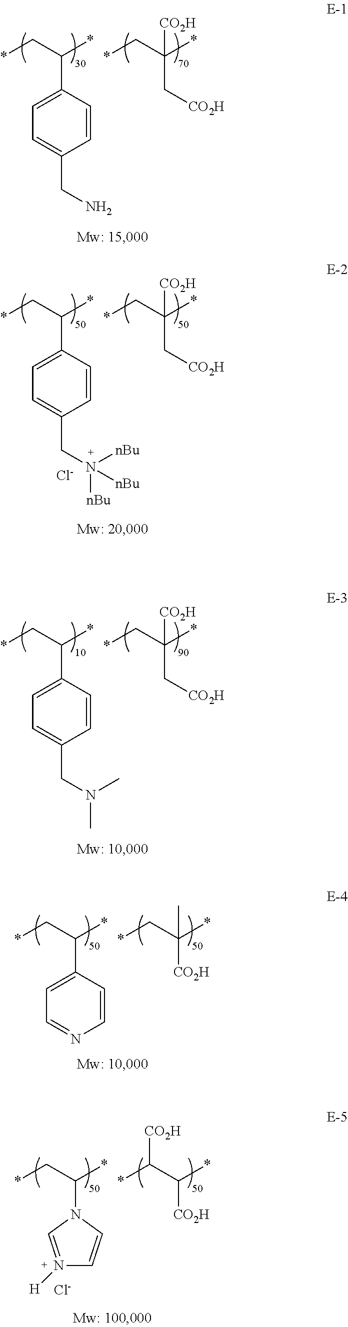

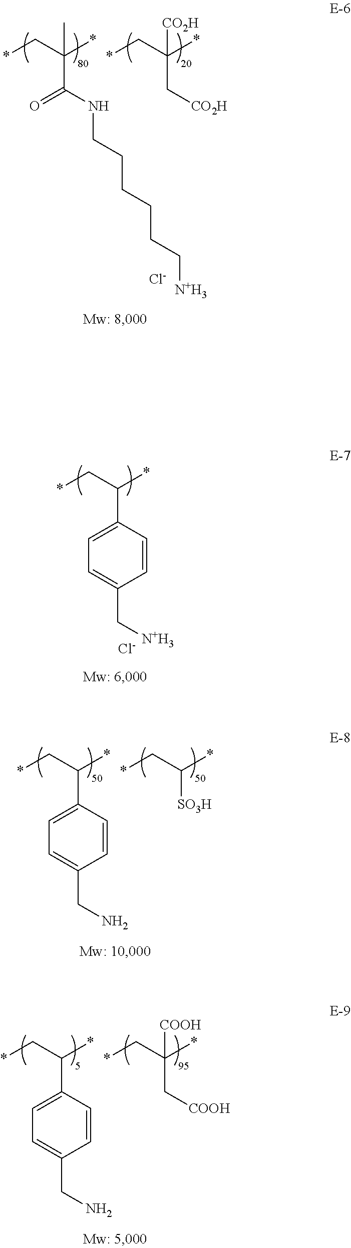

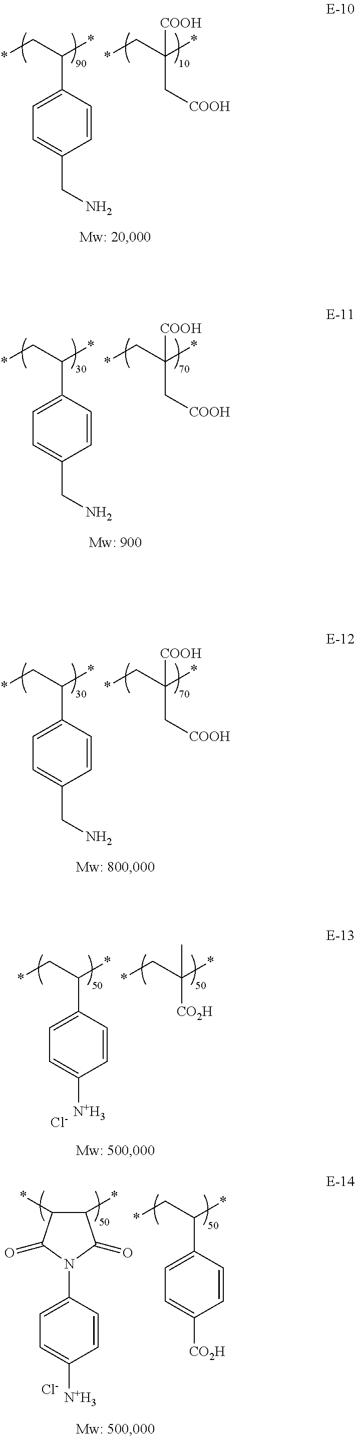

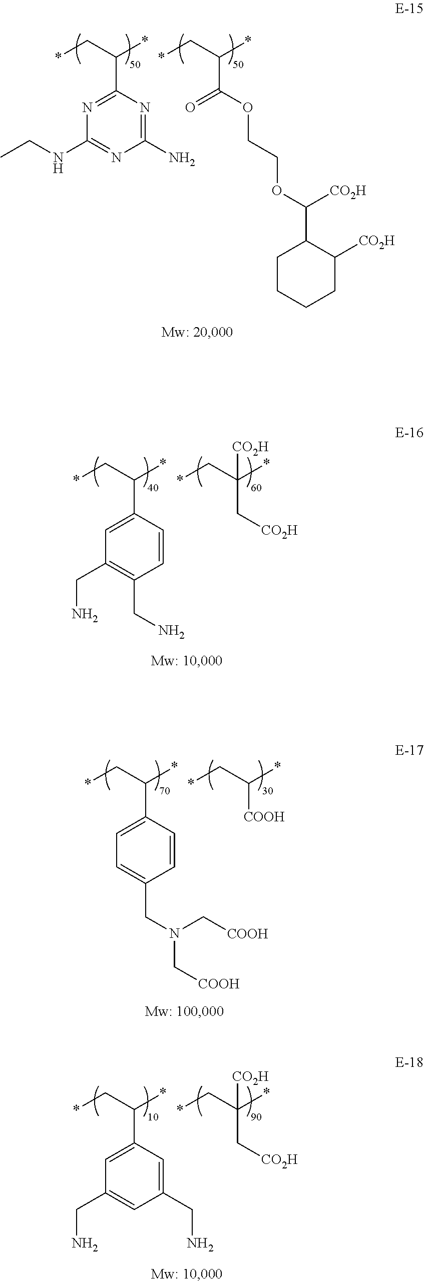

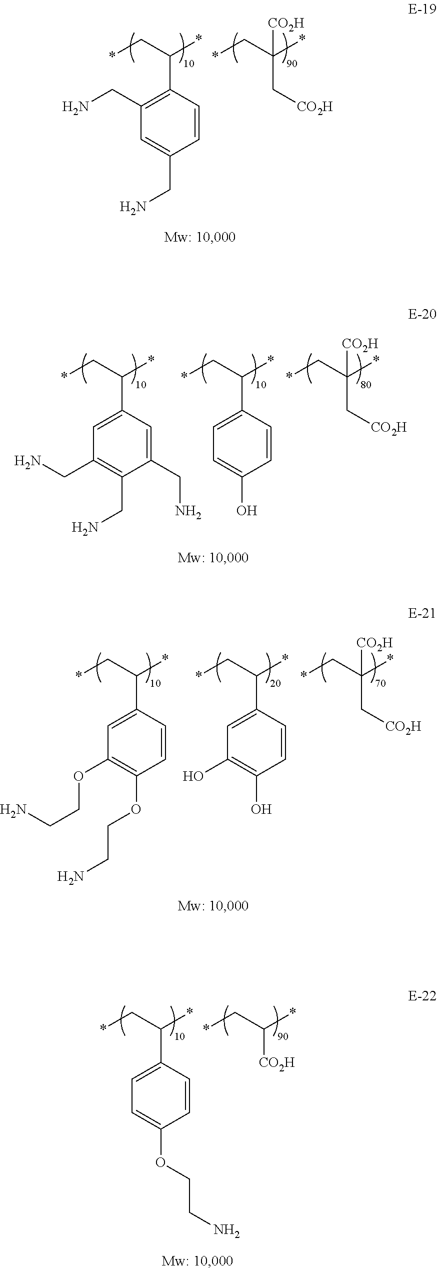

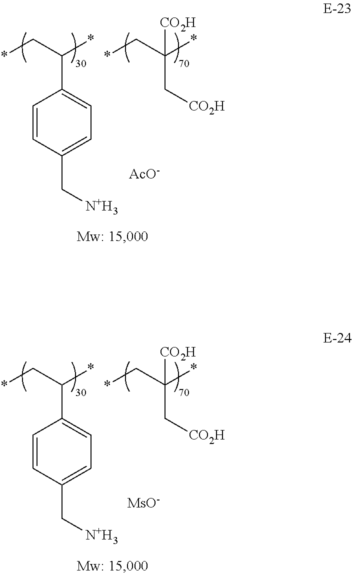

Among these, at least one selected from the group consisting of resins represented by Formula (E-1) and Formulae (E-6) to (E-24) is preferable, and a resin represented by Formula (E-1), Formula (E-6), Formula (E-16), or Formula (E-20) is more preferable.

The weight-average molecular weight Mw of the specific resin is not particularly limited, and it is, for example, 500 to 1,000,000. Among the above, the weight-average molecular weight Mw is preferably 1.000 or more, more preferably 2.000 or more, and particularly preferably 3,000 or more from the viewpoint that the effect of the present invention is more excellent. In addition, the weight-average molecular weight Mw is preferably 500,000 or less, more preferably 100,000 or less, and particularly preferably 50,000 or less from the viewpoint that the solubility of the composition in the post-treatment liquid is more excellent.

One kind of the specific resin may be used alone, or two or more kinds thereof may be used in combination.

The content of the specific resin is preferably 1 ppm by mass to 10% by mass, more preferably 10 to 10,000 ppm by mass (1% by mass), and still more preferably 50 to 1,000 ppm by mass with respect to the total mass of the composition.

In addition, the content of the specific resin is preferably 0.1% to 10.0% by mass and more preferably 0.3% to 3.0% by mass with respect to the total solid content in the composition.

### [Water]

The present composition contains water.

The content of water is not particularly limited; however, it is, for example, 1% to 97% by mass, preferably 10% to 98% by mass, and more preferably 10% to 97% by mass, with respect to the total mass of the composition.

The water is preferably ultrapure water that is used for manufacturing a semiconductor device.

In particular, the water is preferably water in which inorganic anions, metal ions, and the like are reduced. Among the above, it is more preferably water in which the concentration of ions derived from metal atoms of Fe, Co, Na, K, Ca, Cu, Mg, Mn, Li, Al, Cr, Ni, and Zn is reduced, and it is still more preferably water in which the above concentration thereof is adjusted to be on the order of ppt or less (in one form, the metal content is less than 0.001 ppt by mass) at the time when used in the preparation of the composition. The method of carrying out the adjustment is preferably, purification using a filtration membrane or an ion-exchange membrane or purification by distillation. Examples of the method of carrying out the adjustment include the method described in paragraphs [0074] to [0084] of JP2011-110515A and the method described in JP2007-254168A.

It is noted that the water that is used in the embodiment of the present invention is preferably water in which the content of each ion is adjusted as described above. In addition, from the viewpoint that the desired effect of the present invention can be remarkably obtained, it is more preferable that the above-described water is used not only for the composition but also for washing a storage container. In addition, it is preferable that the above-described water is also used in a step of producing the composition, measurement of components of the composition, measurement for evaluation of the composition, and the like.

[Optional component]

The composition may further contain a component other than the above-described components.

Examples of the component which may be contained in the composition include a removing agent, an oxidizing agent, a corrosion inhibitor, a surfactant, an antifoaming agent, and an organic solvent.

### <Removing Agent>

The present composition may contain a removing agent, and it is preferable to contain a removing agent from the viewpoint of further improving the residue removal performance.

The removing agent is not particularly limited as long as it is a compound having a function of removing residues such as an etching residue and an ashing residue, and examples thereof include a fluorine-containing compound, a hydroxylamine compound, a basic compound, and an acidic compound.

### (Fluorine-Containing Compound)

The fluorine-containing compound is not particularly limited as long as it is a compound containing a fluorine atom, and it may be an inorganic compound containing a fluorine atom or may be an organic compound containing a fluorine atom.

Examples of the fluorine-containing compound include hydrofluoric acid (fluorinated acid), ammonium fluoride, tetramethylammonium fluoride, and tetrabutylammonium fluoride. The fluorine-containing compound has a function of removing residues in the composition. As a result, in a case where the composition includes a fluorine-containing compound, the residue removability is more excellent.

The fluorine-containing compound is preferably hydrofluoric acid, ammonium fluoride, or tetramethylammonium fluoride, and it is more preferably hydrofluoric acid or ammonium fluoride.

One kind of the fluorine-containing compound may be used alone, or two or more kinds thereof may be used in combination.

In the composition, the content of the fluorine-containing compound is preferably 0.01% to 15.0% by mass and more preferably 0.1% to 10.0% by mass with respect to the total mass of the composition.

### (Hydroxylamine Compound)

The composition may contain a hydroxylamine compound as a removing agent.

The hydroxylamine compound is at least one compound selected from the group consisting of hydroxylamine (NH₂OH), a hydroxylamine derivative, and a salt thereof.

Since the hydroxylamine compound has a function of promoting decomposition and solubilization of residues and removing residues such as an etching residue and an ashing residue, the composition preferably contains a hydroxylamine compound as the removing agent.

The hydroxylamine derivative is not particularly limited; however, examples thereof include O-methylhydroxylamine, O-ethylhydroxylamine, N-methylhydroxylamine, N,N-dimethylhydroxylamine, N,O-dimethylhydroxylamine, N-ethylhydroxylamine, N,N-diethylhydroxylamine, N,O-diethylhydroxylamine, O,N,N-trimethylhydroxylamine, N,N-dicarboxyethylhydroxylamine, and N,N-disulfoethylhydroxylamine.

Examples of the salts of the hydroxylamine and the hydroxylamine derivative include inorganic acid salts and organic acid salts, where an inorganic acid salt formed by bonding a non-metal atom such as Cl, S, N, or P to a hydrogen atom is preferable, and a salt of any acid of hydrochloric acid, sulfuric acid, or nitric acid is more preferable.

The inorganic acid salts of the hydroxylamine and the hydroxylamine derivative are preferably hydroxylamine nitrate, hydroxylamine sulfate, hydroxylamine hydrochloride, hydroxylamine phosphate, N,N-diethylhydroxylamine sulfate, N,N-diethylhydroxylamine nitrate, or a mixture thereof.

Examples of the organic acid salts of the hydroxylamine and the hydroxylamine derivative include a hydroxylammonium citrate, a hydroxylammonium oxalate, and a hydroxylammonium fluoride.

The hydroxylamine compound is preferably hydroxylamine or hydroxylamine sulfate from the viewpoint of more excellent residue removability.

One kind of the hydroxylamine compound may be used alone, or two or more kinds thereof may be used in combination.

The content of the hydroxylamine compound is not particularly limited; however, it is preferably 0.01% to 30% by mass and more preferably 0.5% to 25% by mass with respect to the total mass of the composition.

### (Basic Compound)

The composition may contain a basic compound as a removing agent. The basic compound is intended to be a compound, where the pH of a solution of the compound exceeds 7 in a case of being dissolved in water. The basic compound also has a function as a pH adjusting agent for adjusting the pH of the composition.

It is noted that in the present specification, the compound contained in a corrosion inhibitor described below is not included in the basic compound.

The basic compound may form a salt with an acid group contained in the repeating unit B of the specific resin.

The basic compound is not particularly limited, and examples thereof include ammonium hydroxide, a water-soluble amine, and a quaternary ammonium compound.

Hereinafter, the ammonium hydroxide, the water-soluble amine, and the quaternary ammonium compound will be described in detail.

The composition may contain ammonium hydroxide (NH₄OH) as the basic compound.

In a case where the composition contains ammonium hydroxide, the content of the ammonium hydroxide is not particularly limited; however, it is preferably 0.01% to 15.0% by mass, and more preferably 0.05% to 10.0% by mass with respect to the total mass of the composition.

The composition may contain a water-soluble amine as a basic compound. In the present specification, the water-soluble amine is intended to be a compound having an amino group in the molecule, where an amount of 50 g or more of the compound can be dissolved in 1 L of water.

Examples of the water-soluble amine include a primary amine having a primary amino group in the molecule, a secondary amine having a secondary amino group in the molecule, a tertiary amine having a tertiary amino group in the molecule, and a salt thereof.

Examples of the salt of the above amine include a salt of an inorganic acid, in which at least one non-metal selected from the group consisting of Cl, S, N, and P is bonded to hydrogen, where a hydrochloride, a sulfate, or a nitrate is preferable.

The water-soluble amine is preferably a low-molecular-weight compound. In the present specification, the “low-molecular-weight compound” means a compound having substantially no molecular weight distribution. The molecular weight of the low-molecular-weight compound is preferably 1,000 or less. Any of following specific examples of the water-soluble amines is a low-molecular-weight compound having a molecular weight of 1,000 or less.

The water-soluble amine may be an alicyclic amine compound having a ring structure in the molecule or may be an alkanol amine having at least one hydroxyalkyl group in the molecule.

Examples of the alicyclic amine compound include 1,8-diazabicyclo[5.4.0]-7-undecene (DBU), F-caprolactam, the following compound 1, the following compound 2, the following compound 3,1,4-diazabicyclo[2.2.2]octane (DABCO), tetrahydrofurfurylamine, N-(2-aminoethyl)piperazine, hydroxyethylpiperazine, piperazine, 2-methylpiperazine, trans-2,5-dimethylpiperazine, cis-2,6-dimethylpiperazine, 2-piperidinemethanol, cyclohexylamine, and 1,5-diazabicyclo[4,3,0]-5-nonene.

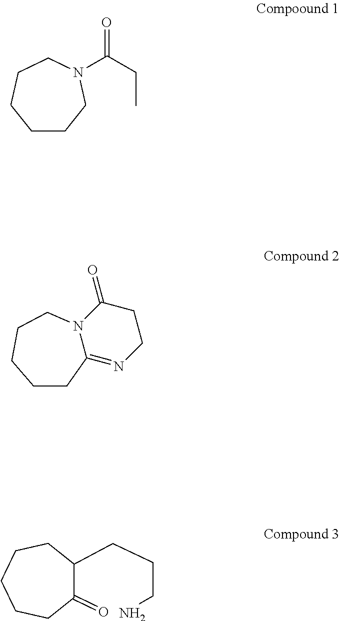

The alkanol amine may have any of a primary amino group, a secondary amino group, or a tertiary amino group; however, it preferably has a primary amino group.

Examples of the alkanol amine include monoethanolamine (MEA, 2-aminoethanol), diethanolamine (DEA), triethanolamine (TEA), diethyleneglycolamine (DEGA), trishydroxymethylaminomethane (Tris), 2-amino-2-methyl-1-propanol (AMP), 2-amino-2-methyl-1,3-dipropanol (AMPD), 2-amino-2-ethyl-1,3-dipropanol (AEPD), 2-(methylamino)-2-methyl-1-propanol (N-MAMP), 2-(dimethylamino)-2-methyl-1-propanol, 2-(aminoethoxy)ethanol (AEE), 2-(2-aminoethylamino)ethanol (CAS registration number: 111-41-1) (AEEA), and N-(3-aminopropyl)diethanolamine (APDA), and Tris is preferable.

Examples of the water-soluble amine other than the alicyclic amine compound and the alkanol amine include primary amines such as methylamine, ethylamine, propylamine, butylamine, pentylamine, methoxyethylamine, methoxypropylamine, 2-amino-2-methyl-1-propanol (AMP), and allylamine; secondary amines such as dimethylamine, diethylamine, dipropylamine, and dibutylamine (DBA); and tertiary amines such as trimethylamine, triethylamine, and tributylamine (TBA).

One kind of the water-soluble amine may be used alone, or two or more kinds thereof may be used in combination.

The content of the water-soluble amine is not particularly limited; however, it is preferably 0.01% to 10% by mass and more preferably 0.1% to 5.0% by mass with respect to the total mass of the composition.

The composition may include, as a removing agent, a compound having one quaternary ammonium cation group in the molecule or a quaternary ammonium compound that is a salt of the compound.

The quaternary ammonium compound is not particularly limited as long as it is a compound having at least one quaternary ammonium cationic group in which the nitrogen atom is substituted with four hydrocarbon groups (preferably an alkyl group), or a salt thereof.

Examples of the quaternary ammonium compound include a quaternary ammonium hydroxide, a quaternary ammonium fluoride, a quaternary ammonium bromide, a quaternary ammonium iodide, a quaternary ammonium acetate, and a quaternary ammonium carbonate.

The quaternary ammonium compound is preferably a quaternary ammonium hydroxide and more preferably a compound represented by Formula (a1).

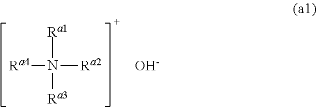

In Formula (a1), R^{a1} to R^{a4} each independently represent an alkyl group having 1 to 16 carbon atoms, an aryl group having 6 to 16 carbon atoms, an aralkyl group having 7 to 16 carbon atoms, or a hydroxyalkyl group having 1 to 16 carbon atoms. At least two of R^{a1} to R^{a4} may be bonded to each other to form a cyclic structure.

From the viewpoint of ease of availability, the compound represented by Formula (a1) is preferably at least one selected from the group consisting of tetramethylammonium hydroxide (TMAH), tetraethylammonium hydroxide (TEAH), tetrapropylammonium hydroxide, tetrabutylammonium hydroxide (TBAH), methyltripropylammonium hydroxide, methyltributylammonium hydroxide, ethyltrimethylammonium hydroxide, dimethyldiethylammonium hydroxide, benzyltrimethylammonium hydroxide (BzTMAH), hexadecyltrimethylammonium hydroxide, (2-hydroxyethyl)trimethylammonium hydroxide, tris(2-hydroxyethyl)methylammonium hydroxide, and spiro-(1,1′)-bipyrrolidinium hydroxide, more preferably TMAH, TEAH, TBAH, or BzTMAH, and still more preferably TMAH, TEAH, or TBAH.

One kind of the quaternary ammonium compound may be used alone, or two or more kinds thereof may be used in combination.

The content of the quaternary ammonium compound is preferably 0.01% to 15% by mass and more preferably 0.1% to 10% by mass with respect to the total mass of the composition.

One kind of the basic compound may be used alone, or two or more kinds thereof may be used in combination.

In a case where the composition contains a basic compound, the content of the basic compound is preferably 0.01% to 20% by mass and more preferably 0.01% to 10% by mass with respect to the total mass of the composition.

### (Acidic Compound)

The composition may contain an acidic compound as the removing agent. The acidic compound is intended to be a compound, where the pH of a solution of the compound becomes less than 7 in a case of being dissolved in water. The acidic compound also has a function as a pH adjusting agent for adjusting the pH of the composition.

It is noted that in the present specification, a compound included in any of the oxidizing agent described below or the anionic surfactant described below is not included in the acidic compound.

The acidic compound may be an inorganic acid or an organic acid.

As described above, the inorganic acid or the organic acid may form a salt with an amino group contained in the repeating unit A of the specific resin.

Examples of the inorganic acid include sulfuric acid, hydrochloric acid, and phosphoric acid, where sulfuric acid is preferable.

One kind of the inorganic acid may be used alone, or two or more kinds thereof may be used in combination.

In a case where the composition contains an inorganic acid, the content of the inorganic compound is preferably 0.01% to 20% by mass and more preferably 0.01% to 10% by mass with respect to the total mass of the composition.

The organic acid is an organic compound that has an acidic functional group and is acidic (has a pH of less than 7.0) in an aqueous solution. Examples of the acidic functional group include a carboxy group, a phosphonate group, a sulfo group, and a phenolic hydroxyl group.

The organic acid is not particularly limited; however, examples thereof include a carboxylic acid having a carboxy group in the molecule (an organic carboxylic acid), a phosphonic acid having a phosphonic acid group in the molecule (an organic phosphonic acid), and a sulfonic acid having a sulfo group in the molecule (an organic sulfonic acid), where a carboxylic acid or is preferable.

The number of acidic functional groups contained in the organic acid is not particularly limited; however, it is preferably 1 to 4 and more preferably 1 to 3.

In addition, the organic acid is preferably a compound having a function of chelating with a metal included in the residue, and it is more preferably a compound having two or more functional groups (coordinating groups) that forms a coordinate bond with a metal ion in the molecule. Examples of the coordinating group include the above-described acidic functional group and amino group.

Examples of the carboxylic acid include a polyamino polycarboxylic acid, an amino acid, a polycarboxylic acid, and a monocarboxylic acid.

The polyamino polycarboxylic acid is a compound having a plurality of amino groups and a plurality of carboxy groups in one molecule, and examples thereof include a mono- or polyalkylene polyamine carboxylic acid, a polyaminoalkane carboxylic acid, a polyaminoalkanol carboxylic acid, and a hydroxyalkyl ether polyamine carboxylic acid.

More specific examples of the polyamino polycarboxylic acid include butylenediaminetetraacetic acid, diethylenetriaminepentaacetic acid (DTPA), ethylenediaminetetrapropionic acid, triethylenetetraminehexacetic acid, 1,3-diamino-2-hydroxypropane-N,N,N′,N′-tetraacetic acid, propylenediaminetetraacetic acid, ethylenediaminetetraacetic acid (EDTA), trans-1,2-diaminocyclohexanetetraacetic acid (Cy-DTA), ethylenediaminediacetic acid, ethylenediaminedipropionic acid, 1,6-hexamethylene-diamine-N,N,N′,N′-tetraacetic acid, N,N-bis(2-hydroxybenzyl)ethylenediamine-N,N-diacetic acid, diaminopropanetetraacetic acid, 1,4,7,10-tetraazacyclododecane-tetraacetic acid, diaminopropanoltetraacetic acid, and (hydroxyethyl)ethylenediaminetriacetic acid.

The amino acid is a compound that has one or more amino groups and one or more carboxy groups in one molecule. However, the above-described polyamino polycarboxylic acid is not included in the amino acid.

Examples of the amino acid include glycine, allylglycine, serine, α-alanine (2-aminopropionic acid), β-alanine (3-aminopropionic acid), lysine, leucine, isoleucine, cystine, cysteine, methionine, ethionine, threonine, tryptophan, tyrosine, valine, histidine, a histidine derivative, asparagine, glutamine, arginine (L-arginine), proline, phenylalanine, the compounds described in paragraphs [0021] to [0023] of JP2016-086094A, and salts thereof. It is noted that as the histidine derivative, the compounds described in JP2015-165561A and JP2015-165562A, the contents of which are incorporated in the present specification by reference, can be used. In addition, examples of the salt include alkali metal salts such as a sodium salt and a potassium salt, an ammonium salt, a carbonate, and an acetate.

In addition, examples of the amino acid having a plurality of amino groups and one carboxy group include a compound represented by Formula (I) or a salt thereof.

(R³NH)C(R¹)(R²)CO₂H (I)

In Formula (I), R¹ and R² each independently represent a hydrogen atom, an alkyl group having 1 to 4 carbon atoms, or a group having at least one nitrogen-containing group, and R³ represents a hydrogen atom, an alkyl group having 1 to 10 carbon atoms, or a group having at least one nitrogen-containing group.

However, at least one of R¹, R², or R³ represents a group having at least one nitrogen-containing group.

Examples of the compound represented by Formula (I) include the following compounds.

Compounds in which R¹ represents a group having at least one nitrogen-containing group and R² and R³ represent hydrogen atoms: lysine, 2,3-diaminobutyric acid, 2,4-diaminobutyric acid, ornithine, 2,3-diaminopropionic acid, 2,6-diaminoheptanoic acid, 4-methyllysine, 3-methyllysine, 5-hydroxylysine, 3-methyl-L-arginine, L-arginine, homoarginine, N⁵-monomethyl-L-arginine, N⁵-[imino(methylamino)methyl]-D-ornithine, canavanine, and histidine.

Compounds in which R¹ and R² represent a hydrogen atom and R³ represents a group having at least one nitrogen-containing group: N-(2-aminoethyl)glycine and N-(2-aminopropyl)glycine.

Compounds in which R¹ represents a group having at least one nitrogen-containing group, R² represents a hydrogen atom, and R³ represents an alkyl group having 1 to 10 carbon atoms: N²-methyllysine and N²-methyl-L-arginine.

Compounds in which R¹ and R³ represent a group having at least one nitrogen-containing group and R² represents a hydrogen atom: N²-(2-aminoethyl)-D-arginine and N²-(2-aminoethyl)-L-arginine.

Compounds in which R¹ represents an alkyl having 1 to 4 carbon atoms, R² represents a group having at least one nitrogen-containing group, and R³ represents a hydrogen atom: 2-methyllysine and 2-methyl-L-arginine.

The polycarboxylic acid is a compound having a plurality of carboxy groups in one molecule. However, the above-described polyamino polycarboxylic acid is not included in the polycarboxylic acid.

Examples of the polycarboxylic acid include oxalic acid, citric acid, malonic acid, maleic acid, succinic acid, malic acid, tartaric acid, glutaric acid, adipic acid, pimelic acid, sebacic acid, citraconic acid, fumaric acid, and itaconic acid, and where citric acid, succinic acid, or tartaric acid is preferable, and citric acid is more preferable.

The monocarboxylic acid is a compound having only one carboxy group in one molecule. However, the above-described amino acids are not included in the monocarboxylic acid.

Examples of the monocarboxylic acid include lower (having 1 to 4 carbon atoms) aliphatic monocarboxylic acids such as formic acid, acetic acid, propionic acid, and butyric acid.

The phosphonic acid may be a monophosphonic acid having one phosphonate group in the molecule or may be a polyphosphonic acid having two or more phosphonate groups in the molecule.

The number of phosphonate groups contained in the phosphonic acid is preferably 2 to 5, more preferably 2 to 4, and still more preferably 2 or 3.

As the phosphonic acid, the compounds represented by General Formulae [1] to [3] described in paragraphs [0013] to [0023] of WO2013/162020A, the compounds described in paragraphs [0026] to [0036] of WO2018/020878A, or the compounds ((co)polymers) described in paragraphs [0031] to [0046] of WO2018/030006A, the contents of which can be incorporated in the present specification by reference, can be used.

Examples of the phosphonic acid include ethylidene diphosphonic acid, 1-hydroxyethylidene-1,1′-diphosphonic acid (HEDEPO), 1-hydroxypropyridene-1,1′-diphosphonic acid, and 1-hydroxybutylidene-1,1′-diphosphonic acid, ethylaminobis(methylenephosphonic acid), dodecylaminobis(methylenephosphonic acid), nitrilotris(methylenephosphonic acid) (NTPO), ethylenediaminebis(methylenephosphonic acid) (EDDPO), 1,3-propylenediaminebis(methylenephosphonic acid), ethylenediaminetetra(methylenephosphonic acid) (EDTPO), ethylenediaminetetra(ethylenephosphonic acid), 1,3-propylenediaminetetra(methylenephosphonic acid) (PDTMP), 1,2-diaminopropanetetra(methylenephosphonic acid), 1,6-hexamethylenediaminetetra(methylenephosphonic acid), diethylenetriaminepenta(methylenephosphonic acid) (DEPPO), diethylenetriaminepenta(ethylenephosphonic acid), triethylenetetraminehexa(methylenephosphonic acid), and triethylenetetraminehexa(ethylenephosphonic acid).

The sulfonic acid may be a monosulfonic acid having only one sulfo group in the molecule or may be a polysulfonic acid having two or more sulfo groups in the molecule.

The number of sulfo groups contained in the sulfonic acid is preferably 1 or 2 and more preferably 1.

Examples of the sulfonic acid include methanesulfonic acid (MSA), ethanesulfonic acid, isethionic acid (2-hydroxyethanesulfonic acid), benzenesulfonic acid, and p-toluenesulfonic acid (tosylic acid), where methanesulfonic acid or isethionic acid is preferable.

The organic acid preferably has a low molecular weight. Specifically, the molecular weight of the organic acid is preferably 600 or less and more preferably 450 or less. The lower limit thereof is not particularly limited; however, it is preferably 85 or more.

In addition, the number of carbon atoms of the organic acid is preferably 15 or less, more preferably 12 or less, and still more preferably 8 or less. The lower limit thereof is not particularly limited; however, it is preferably 2 or more.

The organic acid is preferably the above-described carboxylic acid, more preferably the above-described polyamino polycarboxylic acid, the above-described amino acid, or the above-described polycarboxylic acid, and still more preferably the above-described amino acid or the above-described polycarboxylic acid.

The organic acid may be used alone or in combination of two or more kinds thereof.

In a case where the composition contains an organic acid, the content of the organic acid is not particularly limited; however, it is preferably 0.001% to 90% by mass and more preferably 0.005% to 70% by mass with respect to the total mass of the composition.

The composition may contain a removing agent other than those described above. Examples of another removing agent include a compound having at least two nitrogen-containing groups and no carboxy group. Specific examples of such a compound include at least one biguanide compound selected from the group consisting of a compound having a biguanide group and a salt thereof.

In addition, as a removing agent, the chelating agent described in JP2017-504190A can also be used, and the content described in this document is incorporated in the present specification.

The removing agent is preferably at least one selected from the group consisting of a fluorine-containing compound, a hydroxylamine compound, a basic compound, and an acidic compound, and it is more preferably at least one selected from the group consisting of hydrofluoric acid, ammonium fluoride, a hydroxylamine compound, ammonium hydroxide, a water-soluble amine, a quaternary ammonium compound, sulfuric acid, and carboxylic acid.

In addition, from the viewpoint of further improving the removal performance for dry etching residues in a case of being used as a washing solution, the composition preferably contains, as the removing agent, at least one selected from the group consisting of hydrofluoric acid, a hydroxylamine compound, ammonium hydroxide, a water-soluble amine, and a quaternary ammonium compound, and more preferably contains hydroxylamine, ammonium hydroxide, TMAH, or hydrofluoric acid.

One kind of the removing agent may be used alone, or two or more kinds thereof may be used in combination.

The combination of two or more kinds of removing agents is preferably a combination of at least one basic compound and at least one selected from the group consisting of a hydroxylamine compound and an acidic compound, and more preferably a combination of at least one selected from the group consisting of an alkanol amine and a quaternary ammonium compound and at least one selected from the group consisting of a polyamino polycarboxylic acid and a polycarboxylic acid.

Among those, the acidic compound to be combined with the above-described basic compound is still more preferably EDTA, citric acid, tartaric acid, succinic acid, or malic acid, particularly preferably EDTA, citric acid, or tartaric acid, and most preferably EDTA.

The content of the removing agent is not particularly limited; however, it is preferably 0.001% to 90% by mass and more preferably 0.005% to 70% by mass with respect to the total mass of the composition.

In addition, the content of the removing agent is preferably 0.1% to 10.0% by mass and more preferably 0.3% to 3.0% by mass with respect to the total solid content in the composition.

### <Oxidizing Agent>

The composition may contain an oxidizing agent. In a case where the composition is an etchant, the composition preferably contains an oxidizing agent.

Examples of the oxidizing agent include a peroxide such as hydrogen peroxide or peracetic acid, nitric acid, iodic acid, periodic acid, hypochlorous acid, chlorous acid, chloric acid, perchloric acid, persulfuric acid, bichromic acid, permanganic acid, ozone water, a silver (II) salt, and an iron (III) salt such as iron nitrate. The above-described oxidizing agent may form a salt with a counter ion.

The oxidizing agent that is contained in the composition is preferably hydrogen peroxide, nitric acid, peracetic acid, periodic acid, perchloric acid, chloric acid, hypochlorous acid, a cerium ammonium nitrate salt, iron nitrate, or ammonium persulfate, and it is more preferably hydrogen peroxide, nitric acid, peracetic acid, periodic acid, or perchloric acid.

One kind of the oxidizing agent may be used alone, or two or more kinds thereof may be used in combination.

In a case where the composition contains an oxidizing agent, the content of the oxidizing agent is preferably 0.1% to 20% by mass and more preferably 0.5% to 15% by mass with respect to the total mass of the composition.

In addition, the content of the oxidizing agent is preferably 10% to 80% by mass and more preferably 30% to 60% by mass with respect to the total solid content in the composition.

### <Corrosion Inhibitor>

The composition may contain a corrosion inhibitor, and it preferably contains a corrosion inhibitor.

The corrosion inhibitor is not particularly limited as long as it is a compound that has a function of preventing corrosion of a metal-containing layer due to over-etching or the like, by being coordinated to form a film on the surface of the metal-containing layer, and examples thereof include a heteroaromatic compound, a thiol compound, and a catechol compound.

The heteroaromatic compound is not particularly limited as long as it is a compound having a heteroaromatic ring structure in the molecule; however, it is preferably a nitrogen-containing heteroaromatic compound in which at least one of heteroatoms constituting the heteroaromatic ring is a nitrogen atom.

Examples of the nitrogen-containing heteroaromatic compound include an azole compound, a pyridine compound, a pyrazine compound, and a pyrimidine compound, and an azole compound is preferable.

The azole compound is a compound that has a hetero 5-membered ring containing one or more nitrogen atoms and has aromaticity. The number of nitrogen atoms contained in the hetero 5-membered ring of the azole compound is preferably 1 to 4 and more preferably 1 to 3.

The azole compound may have a substituent on the hetero 5-membered ring. Examples of the substituent include a hydroxy group, a carboxy group, a mercapto group, an amino group, an alkyl group having 1 to 4 carbon atoms, which may have an amino group, and a 2-imidazolyl group.

Examples of the azole compound include an imidazole compound in which one of the atoms constituting the azole ring is a nitrogen atom, a pyrazole compound in which two of the atoms constituting an azole ring are nitrogen atoms, and a thiazole compound in which one of the atoms constituting an azole ring is a nitrogen atom and the other is a sulfur atom, a triazole compound in which three of the atoms constituting an azole ring are nitrogen atoms, and a tetrazole compound in which four of the atoms constituting an azole ring are nitrogen atoms.

Examples of the imidazole compound include imidazole, 1-methylimidazole, 2-methylimidazole, 5-methylimidazole, 1,2-dimethylimidazole, 2-mercaptoimidazole, 4,5-dimethyl-2-mercaptoimidazole, 4-hydroxyimidazole, 2,2′-biimidazole, 4-imidazole carboxylic acid, histamine, and benzimidazole.

Examples of the pyrazole compound include 2,4-dimethylthiazole, benzothiazole, and 2-mercaptobenzothiazole.

Examples of the thiazole compound include 2,4-dimethylthiazole, benzothiazole, and 2-mercaptobenzothiazole.

Examples of the triazole compound include 1,2,4-triazole, 3-methyl-1,2,4-triazole, 3-amino-1,2,4-triazole, 1,2,3-triazole, 1-methyl-1,2,3-triazole, benzotriazole, 1-hydroxybenzotriazole, 1-dihydroxypropylbenzotriazole, 2,3-dicarboxypropylbenzotriazole, 4-hydroxybenzotriazole, 4-carboxybenzotriazole, 5-methyl-1H-benzotriazole, and 2,2′-{[(5-methyl-1H-benzotriazole-1-yl)methyl]imino}diethanol. Among these, benzotriazole, 5-methyl-1H-benzotriazole, or tolyl triazole is preferable, and 5-methyl-1H-benzotriazole is more preferable.

Examples of the tetrazole compound include 1H-tetrazole (1,2,3,4-tetrazole), 5-methyl-1,2,3,4-tetrazole, 5-amino-1,2,3,4-tetrazole, 1,5-pentamethylenetetrazole, 5-mercapto-1-phenyltetrazole, and 1-(2-dimethylaminoethyl)-5-mercaptotetrazole. Among these, 5-mercapto-1-phenyltetrazole is preferable.

The pyridine compound is a compound which contains one nitrogen atom and has a hetero 6-membered ring (pyridine ring) having aromaticity, the pyrazine compound is a compound which has aromaticity and has a hetero 6-membered ring (pyrazine ring) containing two nitrogen atoms located at the para position, and the pyrimidine compound is a compound which has aromaticity and has a hetero 6-membered ring (pyrimidine ring) containing two nitrogen atoms located at the meta position.

Examples of the pyridine compound include pyridine, 3-aminopyridine, 4-aminopyridine, 3-hydroxypyridine, 4-hydroxypyridine, 2-acetamidopyridine, 2-cyanopyridine, 2-carboxypyridine, and 4-carboxypyridine.

Examples of the pyrazine compound include pyrazine, 2-methylpyrazine, 2,5-dimethylpyrazine, 2,3,5-trimethylpyrazine, 2,3,5,6-tetramethylpyrazine, 2-ethyl-3-methylpyrazine, and 2-amino-5-methylpyrazine.

Examples of the pyrimidine compound include pyrimidine, 2-methylpyrimidine, 2-aminopyrimidine, and 4,6-dimethylpyrimidine.

The thiol compound means a compound having at least one thiol group and a hydrocarbon group.

The number of thiol groups contained in the thiol compound is not particularly limited; however, it is preferably 1 or 2 and more preferably 1.

Examples of the hydrocarbon group contained in the thiol compound include an alkyl group (preferably having 4 to 20 carbon atoms), an alkenyl group (preferably having 4 to 12 carbon atoms), an alkynyl group (preferably having 4 to 12 carbon atoms), an aryl group (preferably having 6 to 14 carbon atoms), and an aralkyl group (preferably having 7 to 16 carbon atoms).

The hydrocarbon group may have a substituent. Examples of the substituent include a hydroxyl group, a carboxy group, and an amino group which may have an alkyl group.

Examples of the thiol compound include 1-octanethiol, n-dodecyl mercaptan (1-dodecanethiol), β-mercaptopropionic acid, methyl-3-mercaptopropionate, 2-ethylhexyl-3-mercaptopropionate, n-octyl-3-mercaptopropionate, methoxybutyl-3-mercaptopropionate, stearyl-3-mercaptopropionate, thio phenol, thionaphthol, thionaride, and thioanthranol, where n-dodecyl mercaptan is preferable.

The catechol compound means at least one selected from the group consisting of pyrocatechol (benzene-1,2-diol) and a catechol derivative.

The catechol derivative means a compound in which pyrocatechol is substituted with at least one substituent. Examples of the substituent contained in the catechol derivative include a hydroxy group, a carboxy group, a carboxylic acid ester group, a sulfo group, a sulfonic acid ester group, an alkyl group (preferably having 1 to 6 carbon atoms), and an aryl group (preferably a phenyl group). The carboxy group and the sulfo group, which are contained as a substituent in the catechol derivative, may be a salt with a cation. In addition, the alkyl group and the aryl group contained as a substituent in the catechol derivative may further have a substituent.

Examples of the catechol compound include pyrocatechol, 4-tert-butylcatechol, pyrogallol, gallic acid, methyl gallate, 1,2,4-benzenetriol, and Tyrone.

Examples of the corrosion inhibitor other than those described above include 2,4-diamino-6-methyl-1,3,5-triazine, triazine, diamino-methyltriazine, tritolyl phosphate, a phosphate inhibitor, silanes, benzohydroxamic acids, thiourea, 1,1,3,3-tetramethyl urea, urea, urea derivatives, uric acid, potassium ethylxanthogenate, quinoxaline, acetyl pyrrole, histadine, glutathione (reduced type), thiophene, mercaptopyridine N-oxide, thiamine HCl, tetraethylthiuram disulfide, and phenol.

The corrosion inhibitor is preferably a heteroaromatic compound or a thiol compound, and more preferably a triazole compound, a tetrazole compound, or a thiol compound.

One kind of the corrosion inhibitor may be used alone, or two or more kinds thereof may be used in combination.

In a case where the composition contains a corrosion inhibitor, the content of the corrosion inhibitor is preferably 0.001% to 10% by mass, more preferably 0.002% to 5% by mass, and still more preferably 0.03% to 1% by mass with respect to the total mass of the composition. In a case where two or more kinds of the corrosion inhibitors are used in combination, the total amount thereof is preferably within the above-described range.

In addition, the content of the corrosion inhibitor is preferably 0.1% to 10.0% by mass and more preferably 0.5% to 5.0% by mass with respect to the total solid content in the composition.

As the corrosion inhibitor, a corrosion inhibitor of a high-purity grade is preferably used, which is more preferably used by being further purified.

The method of purifying the corrosion inhibitor is not particularly limited. However, for example, a publicly known method such as filtration, ion exchange, distillation, adsorption purification, recrystallization, reprecipitation, sublimation, or purification using a column is used, and this method can be also applied in combination.

### <Surfactant>

The composition may contain a surfactant.

From the viewpoint that the dissolution of the metal film can be further suppressed, it is preferable that the composition contains a surfactant.

The surfactant is not particularly limited as long as it is a compound having a hydrophilic group and a hydrophobic group (lipophilic group) in the molecule, and examples thereof include an anionic surfactant, a cationic surfactant, a nonionic surfactant, and an amphoteric surfactant.

In a large number of cases, the surfactant has a hydrophobic group selected from an aliphatic hydrocarbon group, an aromatic hydrocarbon group, and a combination thereof. The hydrophobic group contained in the surfactant is not particularly limited; however, in a case where the hydrophobic group includes an aromatic hydrocarbon group, it preferably has 6 or more carbon atoms and more preferably has 10 or more carbon atoms. In a case where the hydrophobic group does not include an aromatic hydrocarbon group and is composed only of an aliphatic hydrocarbon group, it preferably has 10 or more carbon atoms and more preferably 12 or more carbon atoms. The upper limit of the number of carbon atoms of the hydrophobic group is not particularly limited; however, it is preferably 20 or less.

### (Anionic Surfactant)

Examples of the anionic surfactant contained in the composition include a phosphoric acid ester-based surfactant, a phosphonic acid-based surfactant, a sulfonic acid-based surfactant, a carboxylic acid-based surfactant, and a sulfuric acid ester-based surfactant.

Examples of the phosphoric acid ester-based surfactants include a phosphoric acid ester (an alkyl phosphoric acid ester and an aryl phosphoric acid ester), a mono- or polyoxyalkylene ether phosphoric acid ester (a mono- or polyoxyalkylene alkyl ether phosphoric acid ester and a mono- or polyoxyalkylene aryl ether phosphoric acid ester), and salts thereof. Among them, at least one selected from the group consisting of an alkyl phosphoric acid ester, a mono- or polyoxyalkyl ether phosphoric acid ester, or a salt thereof is preferable.

Examples of the salt of the phosphoric acid ester-based surfactant include a sodium salt, a potassium salt, an ammonium salt, and an organic amine salt.

The monovalent alkyl group contained in the alkyl phosphoric acid ester and the mono-or polyoxyalkylene alkyl ether phosphoric acid ester include an alkyl group having 6 to 22 carbon atoms, where an n alkyl group having 10 to 20 carbon atoms is preferable.

Examples of the monovalent aryl group contained in the aryl phosphoric acid ester and the mono- or polyoxyalkylene aryl ether phosphoric acid ester include an aryl having 6 to 14 carbon atoms, which may have an alkyl group, where a phenyl group which may have an alkyl group is preferable.

Examples of the divalent alkylene group contained in the mono- or polyoxyalkylene alkyl ether phosphoric acid ester and the mono- or polyoxyalkylene aryl ether phosphoric acid ester include an alkylene group having 2 to 6 carbon atoms, where an ethylene group or a propylene group is preferable, and an ethylene group is more preferable. The number of repetitions of the oxyalkylene group is preferably 1 to 12 and more preferably 1 to 10.

More specific examples of the phosphoric acid ester-based surfactant include an octyl phosphoric acid ester, a lauryl phosphoric acid ester, a tridecyl phosphoric acid ester, a myristyl phosphoric acid ester, a cetyl phosphoric acid ester, a stearyl phosphoric acid ester, a mono- or polyoxyethylene octyl ether phosphoric acid ester, a mono- or polyoxyethylene lauryl ether phosphoric acid ester, and a mono or polyoxyethylene tridecyl ether phosphoric acid ester. Among them, a lauryl phosphoric acid ester or a mono- or polyoxyethylene lauryl ether phosphoric acid ester is preferable.

In addition, as the phosphoric acid ester-based surfactant, the compounds described in paragraphs [0012] to [0019] of JP2011-040502A, the contents of which can be incorporated in the present specification by reference, can also be used.

Examples of the phosphonic acid-based surfactant include an alkyl phosphonic acid and a polyvinyl phosphonic acid, as well as aminomethyl phosphonic acid described in JP2012-057108A.

Examples of the sulfonic acid-based surfactant include an alkylsulfonic acid, an alkylbenzenesulfonic acid, an alkylnaphthalenesulfonic acid, an alkyl diphenyl ether disulfonic acid, an alkylmethyl taurine, a sulfosuccinic acid diester, a polyoxyalkylene alkyl ether sulfonic acid, and a salt thereof.

Specific examples of the sulfonic acid-based surfactant include hexanesulfonic acid, octanesulfonic acid, decanesulfonic acid, dodecanesulfonic acid, toluenesulfonic acid, cumenesulfonic acid, octylbenzenesulfonic acid, dodecylbenzenesulfonic acid (DBSA), dinitrobenzenesulfonic acid (DNBSA), and lauryldodecylphenyl ether disulfonic acid (LDPEDSA), as well as salts thereof, where dodecylbenzenesulfonic acid is preferable.

Examples of the carboxylic acid-based surfactant include an alkylcarboxylic acid, an alkylbenzenecarboxylic acid, a polyoxyalkylene alkyl ether carboxylic acid, and a salt thereof. Specific examples of the carboxylic acid-based surfactant include lauric acid, myristic acid, palmitic acid, stearic acid, polyoxyethylene lauryl ether acetic acid, and polyoxyethylene tridecyl ether acetic acid.

Examples of the sulfuric acid ester-based surfactant include a sulfuric acid ester (an alkyl ether sulfate ester), a polyoxyalkylene ether sulfuric acid ester, and a salt thereof. Specific examples of the sulfuric acid ester-based surfactant include lauryl sulfate, myristyl sulfate, and polyoxyethylene lauryl ether sulfate.

The anionic surfactant is preferably a phosphoric acid ester-based surfactant, a sulfonic acid-based surfactant, a carboxylic acid-based surfactant, or a sulfuric acid ester-based surfactant.

### (Cationic Surfactant)

Examples of the cationic surfactant include primary to tertiary alkylamine salts (for example, monostearylammonium chloride, distearylammonium chloride, and tristearylammonium chloride), a quaternary ammonium salt (for example, dodecyltrimethylammonium chloride), and modified aliphatic polyamines (for example, polyethylene polyamine).

### (Nonionic Surfactant)

Examples of the nonionic surfactant include a polyoxyalkylene alkyl ether, a polyoxyalkylene alkenyl ether, a polyoxyethylene alkyl phenyl ether, a polyoxyalkylene glycol, a polyoxyalkylene monoalkylate, a polyoxyalkylene dialkylate, a bispolyoxyalkylene alkylamide, a sorbitan fatty acid ester, a polyoxyethylene sorbitan fatty acid ester, a polyoxyethylene alkylamine, a glycerin fatty acid ester, an oxyethyleneoxypropylene block copolymer, an acetylene glycol-based surfactant, and an acetylene-based polyoxyethylene oxide.

### (Amphoteric Surfactant)

Examples of the amphoteric surfactant include carboxybetaine (for example, an alkyl-N,N-dimethylaminoacetic acid betaine and an alkyl-N,N-dihydroxyethylaminoacetic acid betaine), sulfobetaine (for example, an alkyl-N,N-dimethylsulfoethyleneammonium betaine), and imidazolinium betaine (for example, a 2-alkyl-N-carboxymethyl-N-hydroxyethyl imidazolinium betaine).

As the surfactant, the compounds described in paragraphs [0092] to [0096] of JP2015-158662A, paragraphs [0045] and [0046] of JP2012-151273A, and paragraphs [0014] to [0020] of JP2009-147389A, the contents of which are incorporated in the present specification by reference, can also be used.

One kind of the surfactant may be used alone, or two or more kinds thereof may be used in combination.

In a case where the composition contains a surfactant, the content thereof is preferably 0.001% to 3% by mass and more preferably 0.005% to 2% by mass with respect to the total mass of the composition from the viewpoint that the effect of the present invention is more excellent.

In addition, the content of the surfactant is preferably 1.0% to 40.0% by mass and more preferably 5.0% to 30.0% by mass with respect to the total solid content in the composition.

### <Antifoaming Agent>

The composition may contain an antifoaming agent. The surfactant may cause foaming depending on the usage. As a result, it is preferable that the composition containing a surfactant contains an antifoaming agent that suppresses the occurrence of foaming, shortens the life of the generated foam, and suppresses foam residue.

The antifoaming agent is not particularly limited as long as the effect of the present invention is not impaired, and examples thereof include a silicone-based antifoaming agent, an acetylenediol-based antifoaming agent, a fatty acid ester-based antifoaming agent, and a long-chain aliphatic alcohol-based antifoaming agent. Among these, a silicone-based antifoaming agent is preferable from the viewpoint that the effect of suppressing foam residue is more excellent.

The antifoaming agent shall not include the compound included in the above-described surfactant.

The silicone-based antifoaming agent is an antifoaming agent consisting of a compound having a polysiloxane structure. Examples of the silicone-based antifoaming agent include polydimethylsiloxane, silicone glycol, and fluorosilicone. Examples of the long-chain aliphatic alcohol-based antifoaming agent include an aliphatic alcohol having 7 to 22 carbon atoms.

Examples of the antifoaming agent available on the market include KM73A, KA-540, KS508, KS531, KM72, KM85, and KF-6701 (all of which are a silicone-based antifoaming agents) manufactured by Shin-Etsu Chemical Co., Ltd.; Surfynol (registered trademark) MD20 (an acetylenediol-based antifoaming agent) manufactured by Evonic Chemicals; BYK-012 (a polydimethylsiloxane-based antifoaming agent), BYK-014 (a polydimethylsiloxane-based antifoaming agent), BYK-019, BYK-020, BYK-025, BYK-080A, BYK-094, BYK-1650, and BYK-1660 (silicone-based antifoaming agents), manufactured by BYK Additives & Instruments; Q-23183A and SH5510 (both are a silicone-based antifoaming agent) manufactured by DuPont Toray Specialty Materials K.K.; SAG30 (a silicone-based antifoaming agent) manufactured by Nippon Unicar Company Limited; and ADEKA NATE series (silicone-based antifoaming agents), manufactured by ADEKA CORPORATION.

One kind of the antifoaming agent may be used alone, or two or more kinds thereof may be used in combination. In a case where the composition contains an antifoaming agent, the content thereof is preferably 0.0001% to 3% by mass and more preferably 0.001% to 2% by mass with respect to the total mass of the composition from the viewpoint that the suppression of foam residue is more excellent.

In addition, the content of the antifoaming agent is preferably 1.0% to 40.0% by mass and more preferably 5.0% to 30.0% by mass with respect to the total solid content in the composition.

### <Organic Solvent>

The composition may contain an organic solvent, and it is preferable to contain an organic solvent.

The organic solvent is preferably a water-soluble organic solvent. The description that the organic solvent is water-soluble is intended to be that water and the organic solvent at 25° C. can be mixed (dissolved) at any ratio.

Examples of the organic solvent include an alcohol-based solvent, a ketone-based solvent, an ester-based solvent, an ether-based solvent (for example, a glycol diether), a sulfone-based solvent, a sulfoxide-based solvent, a nitrile-based solvent, and an amide-based solvent.

These solvents may be water-soluble.

Among them, the composition preferably contains one or more kinds of organic solvents selected from the group consisting of an alcohol-based solvent, a ketone-based solvent, an ester-based solvent, and an ether-based solvent.

Examples of the alcohol-based solvent include an alkanediol (including, for example, an alkylene glycol), an alkoxyalcohol (including, for example, a glycol monoether), a saturated aliphatic monohydric alcohol, an unsaturated non-aromatic monovalent alcohol, and a low molecular weight alcohol containing a ring structure.

Among them, the alcohol-based solvent is preferably a glycol monoether or a saturated aliphatic monohydric alcohol.

Examples of the alkanediol include glycol, 2-methyl-1,3-propanediol, 1,3-propanediol, 2,2-dimethyl-1,3-propanediol, 1,4-butanediol, 1,3-butanediol, 1,2-butanediol, 2,3-butanediol, pinacol, and an alkylene glycol.

Examples of the alkylene glycol include ethylene glycol, propylene glycol, hexylene glycol, diethylene glycol, dipropylene glycol, triethylene glycol, and tetraethylene glycol, where propylene glycol or hexylene glycol is preferable.

Examples of the alkoxyalcohol include 3-methoxy-3-methyl-1-butanol, 3-methoxy-1-butanol, 1-methoxy-2-butanol, and a glycol monoether, where a glycol monoether is preferable. Examples of the glycol monoether include ethylene glycol mono C1-C4 alkyl ethers such as ethylene glycol monomethyl ether, ethylene glycol monoethyl ether, ethylene glycol mono-n-propyl ether, ethylene glycol monoisopropyl ether, and ethylene glycol monobutyl ether; diethylene glycol mono C1-C4 alkyl ethers such as diethylene glycol monomethyl ether, diethylene glycol monoethyl ether, and diethylene glycol monobutyl ether; triethylene glycol mono C1-C4 alkyl ethers such as triethylene glycol monomethyl ether, triethylene glycol monoethyl ether, and triethylene glycol monobutyl ether; and 1-methoxy-2-propanol, 2-methoxy-1-propanol, 1-ethoxy-2-propanol, 2-ethoxy-1-propanol, propylene glycol mono-n-propyl ether, dipropylene glycol monomethyl ether, dipropylene glycol monoethyl ether, dipropylene glycol mono-n-propyl ether, tripropylene glycol monoethyl ether, tripropylene glycol monomethyl ether, ethylene glycol monobenzyl ether, and diethylene glycol monobenzyl ether.

Among them, an ethylene glycol mono C1 to C4 alkyl ether is preferable, and ethylene glycol monomethyl ether, ethylene glycol monoethyl ether, or ethylene glycol monobutyl ether is more preferable.

Examples of the saturated aliphatic monohydric alcohol include methanol, ethanol, n-propyl alcohol, isopropyl alcohol, 1-butanol, 2-butanol, isobutyl alcohol, tert-butyl alcohol, 2-pentanol, t-pentyl alcohol, and hexanol.

Examples of the unsaturated non-aromatic monohydric alcohol include allyl alcohol, propargyl alcohol, 2-butenyl alcohol, 3-butenyl alcohol, and 4-penten-2-ol.

Examples of the low molecular weight alcohol containing a ring structure include tetrahydrofurfuryl alcohol, furfuryl alcohol, and 1,3-cyclopentanediol.

Examples of the ketone-based solvent include acetone, propanone, cyclobutanone, cyclopentanone, cyclohexanone, diacetone alcohol, 2-butanone, 5-hexanedione, 1,4-cyclohexanedione, 3-hydroxyacetophenone, and 1,3-cyclohexanedione.

Examples of the ester-based solvent include ethyl acetate, butyl acetate, a glycol monoester such as ethylene glycol monoacetate or diethylene glycol monoacetate, and a glycol monoether monoester such as propylene glycol monomethyl ether acetate, ethylene glycol monomethyl ether acetate, propylene glycol monoethyl ether acetate, or ethylene glycol monoethyl ether acetate.

Examples of the sulfone-based solvent include sulfolane, 3-methylsulfolane, and 2,4-dimethylsulfolane, where sulfolane is preferable.

Examples of the sulfoxide-based solvent include dimethyl sulfoxide.

Examples of the nitrile-based solvent include acetonitrile.

Examples of the amide-based solvent include N,N-dimethylformamide, 1-methyl-2-pyrrolidone, 2-pyrrolidinone, 1,3-dimethyl-2-imidazolidinone, F-caprolactam, formamide, N-methylformamide, acetamide, N-methylacetamide, N,N-dimethylacetamide, N-methylpropanamide, and hexamethylphosphoric triamide.

One kind of the organic solvent may be used alone, or two or more kinds thereof may be used in combination.

In a case where the composition contains an organic solvent, the content of the organic solvent is preferably 0.1% to 99% by mass and more preferably 1% to 90% by mass with respect to the total mass of the composition.

### <Metal Component>

The composition may contain a metal component.

Examples of the metal component include metal particles and metal ions. For example, in a case of being referred to as the content of the metal component, it indicates the total content of metal particles and metal ions.

The composition may contain either metal particles or metal ions, or it may contain both metal particles and metal ions.

Examples of the metal atom contained in the metal component include an metal atom selected from the group consisting of Ag, Al, As, Au, Ba, Ca, Cd, Co, Cr, Cu, Fe, Ga, Ge, K, Li, Mg, Mn, Mo, Na, Ni, Pb, Sn, Sr, Ti, and Zn.

The metal component may contain one kind of metal atom or two or more kinds thereof.

The metal particle may be a simple body or an alloy or may be present in a form in which the metal is associated with an organic substance.

The metal component may be a metal component unavoidably contained in each component (raw material) contained in the composition or may be a metal component unavoidably contained during the production, storage, and/or transfer of the composition, and it may be added intentionally.

In a case where the composition has a metal component, the content of the metal component is 0.01 ppt by mass to 10 ppm by mass with respect to the total mass of the composition in a large number of cases, and it is preferably 0.1 ppt by mass to 1 ppm by mass and more preferably 0.1 ppt by mass to 100 mass ppb.

It is noted that the kind and content of the metal component in the composition can be measured by a single nano particle inductively coupled plasma mass spectrometry (SP-ICP-MS) method. The SP-ICP-MS method is different from a general SP-ICP-MS method (inductively coupled plasma mass spectrometry) only in data analysis but uses the same apparatus as in the general SP-ICP-MS method. The data analysis of the SP-ICP-MS method can be carried out by commercially available software.

In the ICP-MS method, the content of the metal component to be measured is measured regardless of the existence form thereof. As a result, the total mass of metal particles to be measured and metal ions is quantified as the content of the metal component. On the other hand, in the SP-ICP-MS method, the content of the metal particles can be measured. As a result, the content of the metal ions in the sample can be calculated by subtracting the content of the metal particles from the content of the metal component in the sample.

Regarding a measuring method by the SP-ICP-MS method, it is possible to carry out measurement according to the method described in Examples, for example, by using an Agilent 8800 triple quadrupole inductively coupled plasma mass spectrometry (ICP-MS, for semiconductor analysis, option #200) manufactured by Agilent Technologies, Inc.). As an apparatus other than those described above, NexION350S manufactured by Perkin Elmer, Inc., and Agilent 8900 manufactured by Agilent Technologies, Inc. can also be used.

The method of adjusting the content of each metal component in the composition is not particularly limited. For example, the content of the metal component in the composition can be reduced by carrying out a publicly known treatment of removing a metal from the composition and/or from a raw material containing each component that is used in the preparation of the composition. In addition, the content of the metal component in the composition can be increased by adding a compound containing metal ions to the composition.

The composition may contain an additive other than the above components. Examples of the additive include a fungicide, a rust inhibitor, and a preservative.

It is noted that the content of each of the components (excluding the metal component) in the composition can be measured by a publicly known method such as a gas chromatography-mass spectrometry (GC-MS) method, a liquid chromatography-mass spectrometry (LC-MS) method, or an ion-exchange chromatography (IC) method.

### [Physical Properties of Composition]

<pH>

The pH of the composition is not particularly limited, and it is, for example, 2.0 to 12.0. In a case of being used as a washing solution, the pH of the composition is preferably 3.0 to 10.0. This is because the effect of the present invention is more excellent in the composition that has a pH in the above-described range.

The pH of the composition is a value obtained by carrying out measurement at 25° C. in accordance with JIS Z8802-1984 using a pH meter (for example, manufactured by HORIBA, Ltd., model “F-74”).

### <Coarse Particle>

It is preferable that the composition is substantially free of coarse particles. The coarse particles refer to particles having a diameter of 0.2 μm or more in a case where the shape of the particles is regarded as a sphere. In addition, a case of being substantially free of coarse particles refers to that ten or fewer particles of 0.2 μm or more are present in 1 mL of the composition in a case where the composition is subjected to measurement using a commercially available measuring device in the light scattering type in-liquid particle measuring method.

It is noted that the coarse particles contained in the composition are particles or the like of dirt, dust, organic solids, inorganic solids, and the like contained as impurities in raw materials, and particles of dirt, dust, and organic solids, and inorganic solids brought in as contaminants during the preparation of the composition, which correspond to the particles that are finally present as particles without being dissolved in the composition.

The amount of the coarse particles present in the composition can be measured in the liquid phase using a commercially available measuring device in a light scattering type in-liquid particle measuring method using a laser as a light source.

Examples of the method of removing coarse particles include a treatment such as filtering.

### [Kit and Concentrated Solution]

The raw materials of the composition may be divided into a plurality of parts to be used as a kit for preparing the composition. Although not particularly limited, specific examples of the method using the composition as a kit include an aspect in which a liquid composition containing water and a removing agent as the first liquid is prepared, and a liquid composition containing the specific resin as the second liquid is prepared.

The content of each component contained in the first liquid and the second liquid provided in the kit is not particularly limited; however, the content of each component in the composition prepared by mixing the first liquid and the second liquid is preferably an amount corresponding to the preferred amount described above.

The pH of each of the first liquid and the second liquid provided in the kit is not particularly limited, and it suffices that the pH is adjusted so that the pH of the composition prepared by mixing the first liquid and the second liquid is a desired value.

In addition, the composition may be prepared as a concentrated solution. In this case, a diluent liquid obtained before use by carrying out dilution with a liquid for dilution is used. That is, the kit may be a kit including the composition as a form of a concentrated solution and the liquid for dilution.

The liquid for dilution is preferably a liquid selected from the group consisting of water, isopropanol, a mixed liquid of water and isopropanol, and a solvent containing ammonium hydroxide, more preferably water, isopropanol, or a mixed liquid of water and isopropanol, and still more preferably water.

The dilution ratio of the composition is not particularly limited; however, it is preferably 1 to 2,000 times and more preferably 1 to 100 times.

It is also possible to suitably use a composition (hereinafter, also referred to as a “diluent liquid”) containing each component with an amount obtained by dividing a suitable content of each component (excluding water) contained in the above-described composition by a dilution ratio (for example, 100) in the above-described range.

The suitable content of each component (excluding water) with respect to the total mass of the diluent liquid is an amount obtained, for example, by dividing the amount described as the suitable content of each component with respect to the total mass of the composition before dilution by a dilution ratio in the above-described range (for example, 100).

A specific method of the diluting step of diluting the composition may be carried out according to a composition preparation step described later. For a stirring device and a stirring method, which are used in the diluting step, a publicly known stirring device described in the composition preparation step described later may be used.

### [Use Application]

Next, the use application of the composition involved in the above-described embodiment will be described.

The present composition is a composition for a semiconductor device. In the present specification, “for a semiconductor device” means that it is used in the manufacture of a semiconductor device. The present composition can be used in any step for manufacturing a semiconductor device, and for example, it can be used in a step of treating a semiconductor substrate, which is included in a manufacturing method for a semiconductor device.

More specifically, the composition can be used for treating an insulating film present on a substrate, a resist, an antireflection film, etching residues, ashing residues, and residues derived from resist films such as a photoresist and a metal hard mask, and the like. It is noted that in the present specification, the etching residues, the ashing residues, the residues derived from a resist film, and the like are collectively referred to as residues. In addition, the present composition may be used for an etching treatment for removing a metal-containing substance on a substrate or may be used for treating a substrate after chemical mechanical polishing.

For example, the composition can be used as a pre-wet liquid to be applied on a substrate to improve the coatability of an actinic ray-sensitive or radiation-sensitive composition before the step of forming a resist film using the composition, a washing solution that is used for removing residues that have adhered on a metal layer, a solution (for example, a removal liquid, a stripping liquid, or the like) that is used for removing various resist films for pattern formation, a solution (for example, a removal liquid, a stripper, or the like) that is used for removing a permanent film (for example, a color filter, a transparent insulating film, a lens made of a resin) or the like from a semiconductor substrate, or the like. It is noted that since the semiconductor substrate after the removal of the permanent film may be used again for the semiconductor device, the removal of the permanent film is included in the manufacturing step of the semiconductor device.

In addition, the present composition can also be used as a washing solution that is used for removing residues such as metal impurities or fine particles from a substrate after chemical mechanical polishing.

In addition, the present composition can also be used as an etchant for a metal-containing substance (including a metal oxide and a composite oxide consisting of a plurality of metal oxides) on a substrate.

Among the above-described use applications, in particular, it can be suitably used as a washing solution for removing residues, a solution for removing a resist film used in a pattern formation, a washing solution for removing residues from a substrate after chemical mechanical polishing, or an etchant.

The composition may be used in only one use application or two or more of use applications among the above-described use applications.

In the above-described use applications, a diluent liquid obtained by diluting the composition can also be used. Among these, it can be suitably used as a washing solution for removing residues on a substrate (more preferably, a substrate on which chemical mechanical polishing has been carried out).

The present composition can be suitably used for treating a substrate of a semiconductor device, which includes a metal layer containing W, and a substrate of a semiconductor device, which includes a metal layer containing Mo. The present composition can also be used for treating a substrate of a semiconductor device, which includes a metal layer containing Co, and a substrate of a semiconductor device, which includes a metal layer containing Cu. Further, since the present composition has an excellent corrosion prevention property with respect to an insulating film, it can also be used, for example, for treating a substrate of a semiconductor device which includes a layer containing at least one selected from the group consisting of SiOₓ, SiN, and SiOC (x represents a number in a range of 1 to 3).

### [Production Method for Composition, Concentrated Solution, and Kit]

### <Composition Preparation Step>

A method for producing the present composition is not particularly limited, and the present composition can be produced by a publicly known production method. Examples of the production method for the present composition include a method having at least a composition preparation step of mixing the above-described components to prepare the composition.

In the composition preparation step, the order in which the respective components are mixed is not particularly limited. It is preferable that each of the liquids provided in the kit and the concentrated solution is also produced according to the same method as described above.

The method for producing the kit is not particularly limited. For example, after preparing the first liquid and the second liquid described above, the first liquid and the second liquid may be respectively accommodated in containers different from each other to produce a kit for preparing the composition.

### <Filtration Step>

It is preferable that the production method includes a filtration step of filtering a liquid in order to remove foreign substances, coarse particles, and the like from the liquid.

The filtration method is not particularly limited, and a publicly known filtration method can be used. Among these, filtering using a filter is preferable.

The filter that is used for filtering can be used without particular limitation as long as it is a filter that is conventionally used in the use application of filtering. Examples of the material constituting the filter include a fluororesin such as polytetrafluoroethylene (PTFE), a polyamide-based resin such as nylon, and a polyolefin-based resin (having a high density and an ultrahigh molecular weight) such as polyethylene or polypropylene (PP). Among them, a polyamide-based resin, PTFE, and polypropylene (including high-density polypropylene) are preferable.

In a case of using a filter formed from these materials, it is possible to more effectively remove foreign substances having high polarity, which are likely to cause defects, from the composition.

The lower limit value of the critical surface tension of the filter is preferably 70 mN/m or more, and the upper limit value thereof is preferably 95 mN/m or less. In particular, the critical surface tension of the filter is preferably 75 to 85 mN/m.

It is noted that the value of the critical surface tension is a nominal value of a manufacturer. In a case of using a filter having a critical surface tension in the above range, it is possible to more effectively remove foreign substances having high polarity, which are likely to cause defects, from the composition.

The pore diameter of the filter is preferably about 0.001 to 1.0 μm, more preferably about 0.02 to 0.5 μm, and still more preferably about 0.01 to 0.1 μm. In a case of setting the pore diameter of the filter within the above range, it is possible to reliably remove fine foreign substances contained in the composition while suppressing filtration clogging.

In a case of using a filter, different filters may be combined. In this case, filtering with a first filter may be carried out only once or may be carried out twice or more times. In a case where different filters are combined and filtering is carried out two or more times, the kinds of filters may be the same or different from each other; however, the kinds of filters are preferably different from each other. Typically, it is preferable that at least one of the pore diameter or the constitutional material is different between the first filter and the second filter.

It is preferable that the pore diameters of the second and subsequent filtering are equal to or smaller than the pore diameter of the first filtering. In addition, the first filters having different pore diameters within the above range may be combined. With regard to the pore diameters herein, reference can be made to nominal values of filter manufacturers. A commercially available filter can be selected from various filters provided by, for example, Nihon Pall Ltd., Advantec Toyo Kaisha, Ltd., Nihon Entegris K. K. (formerly Nippon Microlith Co., Ltd.), and Kitz Micro Filter Corporation. In addition, the following filters can also be used: “P-nylon filter (pore diameter: 0.02 μm, critical surface tension: 77 mN/m)” made of polyamide (manufactured by Nihon Pall Ltd.); “PE clean filter (pore diameter: 0.02 μm)” made of high-density polyethylene (manufactured by Nihon Pall Ltd.); and “PE clean filter (pore diameter: 0.01 μm)” made of high-density polyethylene (manufactured by Nihon Pall Ltd.).

As the second filter, a filter formed of the same material as that of the first filter can be used. A filter having the same pore diameter as that of the first filter described above can be used. In a case where the second filter having a pore diameter smaller than that of the first filter is used, the ratio of the pore diameter of the second filter to the pore diameter of the first filter (the pore diameter of the second filter/the pore diameter of the first filter) is preferably 0.01 to 0.99, more preferably 0.1 to 0.9, and still more preferably 0.3 to 0.9. In a case of setting the pore diameter of the second filter within the above range, fine foreign substances mixed in the composition are more reliably removed.

For example, filtering with the first filter may be carried out with a mixed liquid containing a part of components of the composition, and after mixing the remaining components with the mixed liquid to prepare the composition, the filtering with the second filter may be carried out.

In addition, it is preferable that the filter to be used is treated before filtering the composition. The liquid that is used for this treatment is not particularly limited; however, it is preferably the composition or a liquid containing components that are contained in the concentrated solution and the composition.

In a case of carrying out filtering, the upper limit value of the temperature during filtering is preferably room temperature (25° C.) or lower, more preferably 23° C. or lower, and still more preferably 20° C. or lower. The lower limit value of the temperature at the time of filtering is preferably 0° C. or higher, more preferably 5° C. or higher, and still more preferably 10° C. or higher.

In the filtering, particulate foreign substances and/or impurities can be removed. However, in a case where the filtering is carried out at the above temperature, the amount of the particulate foreign substances and/or impurities dissolved in the composition is reduced, and thus the filtering is carried out more efficiently.

### <Destaticization Step>

The above-described production method may further include a destaticization step of destaticizing at least one selected from the group consisting of a composition, a concentrated solution, and a kit. A specific method for destaticization will be described later.

It is preferable that all the steps involved in the production method are carried out in a clean room. It is preferable that the clean room satisfies 14644-1 clean room standards. It is preferable that the clean room satisfies any of the International Organization for Standardization (ISO) Class 1, ISO Class 2, ISO Class 3, or ISO Class 4, it is more preferable that the clean room satisfies ISO Class 1 or ISO Class 2, and it is still more preferable that the clean room satisfies ISO Class 1.

### <Container>

The container that accommodates the above-described composition, concentrated solution, or kit is not particularly limited as long as the corrosiveness due to the liquid does not cause a problem, and a publicly known container can be used.

The container is preferably a container for a use application in a semiconductor, which has high internal cleanliness and hardly causes elution of impurities.

Specific examples of the container include “CLEAN BOTTLE” series manufactured by AICELLO CHEMICAL Co., Ltd. and “PURE BOTTLE” manufactured by KODAMA PLASTICS Co., Ltd. In addition, for the intended purpose of preventing the mixing (contamination) of raw materials and impurities into the chemical liquid, it is also preferable to use a multi-layer container in which an inner wall of the container has a six-layer structure consisting of six kinds of resins and a multi-layer container in which an inner wall of the container has a seven-layer structure consisting of six kinds of resins. Examples of these containers include a container described in JP2015-123351A, which are not limited thereto.

The inner wall of the container is preferably formed of or coated with one or more resins selected from the group consisting of a polyethylene resin, a polypropylene resin, and a polyethylene-polypropylene resin, a resin different from these, and a metal such as stainless steel, HASTELLOY, INCONEL, or MONEL.

As the above-described different resin, a fluorine-based resin (a perfluoro resin) can be preferably used. In this manner, by using a container in which an inner wall of the container is formed of a fluorine-based resin or coated with a fluororesin, the occurrence of a problem of elution of ethylene or propylene oligomers can be suppressed, as compared with a case of using a container in which an inner wall of the container is formed of or coated with a polyethylene resin, a polypropylene resin, or a polyethylene-polypropylene resin.

Specific examples of such a container having an inner wall include a FluoroPure PFA composite drum manufactured by Entegris Inc. In addition, it is also possible to use the containers described on page 4 of the pamphlet of JP1991-502677A (JP-H3-502677A), page 3 of the pamphlet of WO2004/016526A, pages 9 and 16 of the WO99/046309A.

Further, for the inner wall of the container, quartz and an electropolished metal material (that is, a completely electropolished metal material) are also preferably used, in addition to the above-described fluorine-based resin.

The metal material used that is for producing the electropolished metal material is preferably a metal material which contains at least one selected from the group consisting of chromium and nickel, and has a total content of chromium and nickel of more than 25% by mass with respect to the total mass of the metal material, and examples thereof include stainless steel and a nickel-chromium alloy.

The total content of chromium and nickel in the metal material is preferably 25% by mass or more and more preferably 30% by mass or more with respect to the total mass of the metal material.

It is noted that the upper limit value of the total content of chromium and nickel in the metal material is not particularly limited; however, it is generally preferably 90% by mass or less.

The stainless steel is not particularly limited, and publicly known stainless steel can be used. Among them, an alloy containing nickel at 8% by mass or more is preferable, and austenitic stainless steel containing nickel at 8% by mass or more is more preferable. Examples of the austenitic stainless steels include SUS (Steel Use Stainless) 304 (Ni content: 8% by mass, Cr content: 18% by mass), SUS304L (Ni content: 9% by mass, Cr content: 18% by mass), SUS316 (Ni content: 10% by mass, Cr content: 16% by mass), and SUS316L (Ni content: 12% by mass, Cr content: 16% by mass).

The nickel-chromium alloy is not particularly limited, and publicly known nickel-chromium alloys can be used. Among them, a nickel-chromium alloy having a nickel content of 40% to 75% by mass and a chromium content of 1% to 30% by mass is preferable. Examples of the nickel-chromium alloy include HASTELLOY (trade name, the same applies hereinafter), MONEL (trade name, the same applies hereinafter), and INCONEL (trade name, the same applies hereinafter). More specific examples thereof include HASTELLOY C-276 (Ni content: 63% by mass, Cr content: 16% by mass), HASTELLOY-C(Ni content: 60% by mass, Cr content: 17% by mass), and HASTELLOY C-22 (Ni content: 61% by mass, Cr content: 22% by mass).

In addition, the nickel-chromium alloy may further contain boron, silicon, tungsten, molybdenum, copper, cobalt, and the like as necessary, in addition to the above alloys.

A method of electropolishing a metal material is not particularly limited, and publicly known methods can be used. For example, the methods described in paragraphs [0011] to [0014] of JP2015-227501A, paragraphs [0036] to [0042] of JP2008-264929A, or the like can be used.

In a case where the metal material is electropolished, it is presumed that a content of chromium in a passivation layer on a surface becomes larger than a content of chromium in a primary phase. As a result, it is presumed that since the metal element is unlikely to flow out into the composition from the inner wall coated with the electropolished metal material, the composition in which the specific metal element is reduced can be obtained.

The metal material is preferably subjected to buff polishing. A method of buff polishing is not particularly limited, and publicly known methods can be used. The size of abrasive grains for polishing used for buff polishing finish is not particularly limited; however, it is preferably #400 or less from the viewpoint that then unevenness of the surface of the metal material is easily reduced.

The buff polishing is preferably carried out before the electropolishing.

In addition, the metal material may be treated by combining one or two or more of a plurality of stages of buff polishing, acid washing, magnetic fluid polishing, and the like, which are carried out by changing the count of the size or the like of the abrasive grains.

It is preferable to wash the inside of these containers before being filled. The liquid that is used for washing may be appropriately selected according to the use application; however, it is preferably the present composition, a liquid obtained by diluting the present composition, or a liquid containing at least one of the components which are added to the present composition.

In order to prevent the change in the components in the composition during storage, the inside of the container may be replaced with inert gas (nitrogen, argon, or the like) with a purity of 99.99995% by volume or more. In particular, a gas having a low moisture content is preferable. Although the liquid container body may be transported and stored at normal temperature, the temperature may be controlled in a range of −20° C. to 20° C. in order to prevent deterioration.

### [Substrate Treatment Method]

In a substrate treatment method using a composition (hereinafter, also simply referred to as “the present treatment method”), the present composition can be typically used by being brought into contact with a substrate (hereinafter, also referred to as “an object to be treated”) having a metal-containing substance which is a material containing a metal. At this time, the object to be treated may contain a plurality of kinds of metal-containing substances.

### [0207] [Object to be Treated]

The object to be treated, which is an object to be treated using a composition, is not particularly limited as long as it is a substrate having a metal-containing substance.

It is noted that “on the substrate” in the present specification includes, for example, any of the front and back surfaces, the side surface, the inside of the groove, and the like of the substrate. In addition, the case of “the metal-containing substance on the substrate” includes not only a case where the metal-containing substance is directly present on the surface of the substrate but also a case where the metal-containing substance is present on the substrate through another layer.

In addition, the “substrate” in the present specification includes, for example, a semiconductor substrate consisting of a single layer and a semiconductor substrate consisting of multiple layers.

In addition, the notation of “object to be removed” in the present specification is at least one selected from the group consisting of metal-containing substances that are present on the substrate and are targets to be removed using the composition.

The metal-containing substance is a material including a simple body of a metal (a metal atom) as a main component.

Examples of the metal contained in the metal-containing substance include at least one metal M selected from the group consisting of copper (Cu), cobalt (Co), tungsten (W), titanium (Ti), tantalum (Ta), ruthenium (Ru), chromium (Cr), hafnium (Hf), osmium (Os), platinum (Pt), nickel (Ni), manganese (Mn), zirconium (Zr), molybdenum (Mo), lanthanum (La), and iridium (Ir).

The metal-containing substance only needs to be a substance containing a metal (a metal atom), and examples thereof include a substance composed of at least one selected from the group consisting of a simple body of the metal M, an alloy including the metal M, an oxide of the metal M, a nitride of the metal M, and an oxynitride of the metal M.

More specific examples of the metal-containing substance include a metal-containing substance containing at least one component selected from the group consisting of copper, cobalt, a cobalt alloy, tungsten, a tungsten alloy, ruthenium, a ruthenium alloy, tantalum, a tantalum alloy, aluminum oxide, aluminum nitride, aluminum oxynitride, titanium aluminum, titanium, titanium nitride, titanium oxide, zirconium oxide, hafnium oxide, tantalum oxide, lanthanum oxide, and a yttrium alloy.

In addition, the metal-containing substance may be a mixture containing two or more of these compounds.

It is noted that the oxide, the nitride, and the oxynitride may be a composite oxide, a composite nitride, or a composite oxynitride, which includes a metal. In addition, the metal-containing substance may contain at least one element selected from the group consisting of a simple body metal, an alloy, a metal oxide, and a metal nitride, and at least one element, as a dopant, selected from the group consisting of carbon, nitrogen, boron, and phosphorus.

The content of the metal atom in the metal-containing substance is preferably 10% by mass or more, more preferably 30% by mass or more, and still more preferably 50% by mass or more with respect to the total mass of the metal-containing substance. The upper limit thereof is 100% by mass since the metal-containing substance may be the metal itself.

In a case where the metal-containing substance contains the above dopant, the content of the dopant is preferably 0.1% to 50% by mass and more preferably 10% to 40% by mass with respect to the total mass of the metal-containing substance. In that case, the content of the metal atom in the metal-containing substance is preferably 30% to 99.9% by mass and more preferably 60% to 90% by mass with respect to the total mass of the metal-containing substance.

The metal oxide-containing substance is a material containing an oxide of a metal as a main component.

Examples of the metal atom constituting the oxide of the metal contained in the metal oxide-containing substance include the metal M. The metal oxide-containing substance preferably contains an oxide of the metal M and more preferably contains tungsten oxide (WOx), cobalt oxide (CoOx), or ruthenium oxide (RuOx).

The metal oxide-containing substance may be a mixture containing two or more kinds of metals of oxides.

In addition, the metal oxide-containing substance may contain a simple body of a metal and/or a nitride of a metal as long as the oxide of the metal is contained as a main component.

The content of the oxide of the metal in the metal oxide-containing substance is preferably 5% by mass or more and more preferably 20% by mass or more with respect to the total mass of the metal oxide-containing substance. The upper limit thereof is not particularly limited and may be 100% by mass or less.

A form of the metal-containing substance is not particularly limited, and it may be, for example, any of a film-shaped (layer-shaped) form, a wiring line-shaped form, and a particle-shaped form.

The metal-containing substance may be disposed only on one main surface of the substrate or may be disposed on both main surfaces. In addition, the metal-containing substance may be disposed on the entire main surface of the substrate or may be disposed on a part of the main surface of the substrate.

The substrate preferably has a metal M-containing substance containing a metal M, more preferably has a metal-containing substance containing at least one metal selected from the group consisting of W, Mo, Cu, Co, Ti, Ta, and Ru, still more preferably has a metal-containing substance containing at least one metal selected from the group consisting of W, Mo, Cu, and Co, and particularly preferably has a W-containing substance containing at least one of W or a W alloy.

Among these, the substrate preferably has a W-containing film, a Mo-containing film, a copper-containing film, a Co-containing film, or a Ti-containing film, and more preferably has a W-containing film or a Mo-containing film.

Examples of the W-containing film include a metal film (a W metal film) consisting of only tungsten and a metal film (a W alloy metal film) consisting of an alloy made of tungsten and another metal. Specific examples of the W alloy metal film include a WTi alloy metal film and a WCo alloy metal film.

The tungsten-containing film is often used as a wiring line film or a barrier metal.

Examples of the Mo-containing film include a metal film consisting of only molybdenum (an Mo metal film) and a metal film made of an alloy consisting of molybdenum and another metal (an Mo alloy metal film). Specific examples of the Mo alloy metal film include a MoCo alloy metal film.

Examples of the copper-containing film include a wiring line film (a copper wiring line film) consisting of only metallic copper and a wiring line film (a copper alloy wiring line film) consisting of an alloy, consisting of metallic copper and another metal.

Specific examples of the copper alloy wiring line film include a wiring line film consisting of an alloy of one or more metals selected from Al, Ti, Cr, Mn, Ta, and W, and copper. More specific examples of the copper alloy wiring line film include a CuAl alloy wiring line film, a CuTi alloy wiring line film, a CuCr alloy wiring line film, a CuMn alloy wiring line film, a CuTa alloy wiring line film, and a CuW alloy wiring line film.

Examples of the Co-containing film include a metal film (a Co metal film) consisting of only metal cobalt and a metal film (a Co alloy metal film) consisting of an alloy consisting of metallic cobalt and another metal.

Specific examples of the Co alloy metal film include a metal film consisting of an alloy made of one or more metals selected from Ti, Cr, Fe, Ni, Mo, Pd, Ta, and W, and cobalt. More specific examples of the Co alloy metal film include a CoTi alloy metal film, a CoCr alloy metal film, a CoFe alloy metal film, a CoNi alloy metal film, a CoMo alloy metal film, a CoPd alloy metal film, a CoTa alloy metal film, and a CoW alloy metal film.

Among the Co-containing films, the Co metal film is often used as the wiring line film, and the Co alloy metal film is often used as the barrier metal.

Examples of the Ti-containing film include a metal film containing an alloy consisting of Ti, Al, or the like, where the metal film is a Ti alloy metal film which may further contain the above-described dopant. Specific examples of the Ti alloy metal film include a TiAl film, a TiAlC film, and a TiAlN film.

The Ti alloy metal film is often used in a structure of a gate and a structure surrounding the gate.

More specific examples of the object to be treated include a laminate including, on a substrate, a metal layer, an insulating film, and a metal hard mask at least in this order. The laminate may further have holes formed from the surface (the opening portion) of the metal hard mask toward the substrate so that the surface of the metal layer is exposed, as a result of undergoing a dry etching step or the like.

A manufacturing method for such a laminate having holes as described above is not particularly limited; however, examples thereof include a method in which a laminate before treatment, having a substrate, a metal layer, an insulating film, and a metal hard mask in this order, is subjected to a dry etching step by using a metal hard mask as a mask and an insulating film is etched so that the surface of the metal layer is exposed, thereby providing holes that penetrate through the metal hard mask and the inside of the insulating film.

It is noted that a manufacturing method for the metal hard mask is not particularly limited. For example, first, a metal layer containing a predetermined component is formed on an insulating film, and a resist film having a predetermined pattern is formed on the metal layer. Next, a method of manufacturing a metal hard mask (that is, a film in which a metal layer is patterned) by etching a metal layer using a resist film as a mask can be mentioned.

In addition, the laminate may have a layer other than the above-described layers, examples of which include an etching stop film and an antireflection layer.

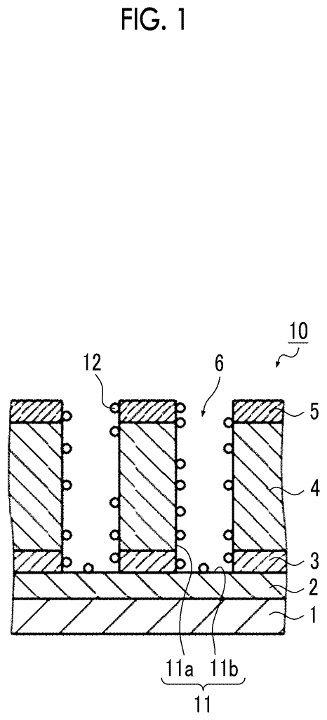

FIG. 1 shows a schematic cross-sectional view illustrating an example of a laminate which is an object to be treated in the present treatment method.

A laminate 10 illustrated in FIG. 1 includes a metal layer 2, an etching stop layer 3, an insulating film 4, and a metal hard mask 5 in this order on a substrate 1, and holes 6 by which the metal layer 2 is exposed is formed at a predetermined position as a result of a dry etching step or the like. That is, the object to be treated illustrated in FIG. 1 is a laminate which includes the substrate 1, the metal layer 2, the etching stop layer 3, the insulating film 4, and the metal hard mask 5 in this order, in which the holes 6 that penetrate from the surface of the metal hard mask 5 to the surface of the metal layer 2 at the position of the opening portion of the metal hard mask 5. An inner wall 11 of the holes 6 is consisting of a cross-sectional wall 11a consisting of the etching stop layer 3, the insulating film 4, and the metal hard mask 5, and a bottom wall 11b consisting of the exposed metal layer 2, and dry etching residues 12 are attached thereto.

The present treatment method can be suitably used for removing these dry etching residues 12. That is, while being excellent in the removal performance (the residue removability) for the dry etching residues 12, it is also excellent in the corrosion prevention property with respect to the inner wall 11 (for example, the metal layer 2 and the like) of the object to be treated.

In addition, a laminate that has undergone a dry ashing step after the dry etching step may be subjected to the substrate treatment method.

Hereinafter, each layer-constituting material of the above-described laminate will be described.

### <Metal Hard Mask>

The metal hard mask preferably contains at least one component selected from the group consisting of copper, cobalt, a cobalt alloy, tungsten, a tungsten alloy, ruthenium, a ruthenium alloy, tantalum, a tantalum alloy, aluminum oxide, aluminum nitride, aluminum oxynitride, titanium aluminum, titanium carbide aluminum, titanium, titanium nitride, titanium oxide, zirconium oxide, hafnium oxide, tantalum oxide, lanthanum oxide, and a yttrium alloy (preferably YSiOx). Here, x and y are preferably numbers represented by x=1 to 3 and y=1 to 2, respectively.

Examples of the material of the metal hard mask include TiN, TiAl, TiAlC, WO₂, and ZrO₂.

### <Insulating Film>

The material of the insulating film is not particularly limited, and examples thereof include those having a dielectric constant k of 3.0 or less and more preferably 2.6 or less.

Specific examples of the material of the insulating film include organic polymers such as SiOₓ, SiN, SiOC, and polyimide. Here, x is preferably a number represented by 1 to 3.

The insulating film may be composed of a plurality of films. Examples of the insulating film composed of a plurality of films include an insulating film obtained by combining a film containing silicon oxide and a film containing oxidized silicon carbide.

### <Etching Stop Layer>

The material of the etching stop layer is not particularly limited. Specific examples of the material of the etching stop layer include materials based on SiN, SiON, and SiOCN, and metal oxides such as AlOx.

### <Metal Layer>

The material that forms the metal layer, which is a wiring line material and/or a plug material, is not particularly limited. However, it preferably contains one or more selected from the group consisting of cobalt, tungsten, and copper. Further, the material that forms the metal layer may be cobalt, tungsten, or an alloy of copper and another metal.

The metal layer may further contain a metal other than cobalt, tungsten, and copper, a metal nitride, and/or an alloy. Examples of the metal other than cobalt, tungsten, and copper, which may be contained in the metal layer include titanium, titanium-tungsten, titanium nitride, tantalum, a tantalum compound, chromium, a chromium oxide, and aluminum.

The metal layer may contain at least one dopant selected from the group consisting of carbon, nitrogen, boron, and phosphorus, in addition to one or more selected from the group consisting of cobalt, tungsten, and copper.

### <Substrate>

Specific examples of the wafer constituting a substrate include a wafer consisting of silicon-based material, such as a silicon (Si) wafer, a silicon carbide (SiC) wafer, or a wafer based on a resin containing silicon (a glass epoxy wafer), a gallium phosphide (GaP) wafer, a gallium arsenide (GaAs) wafer, and an indium phosphide (InP) wafer.

The silicon wafer may be an n-type silicon wafer obtained by doping a silicon wafer with a pentavalent atom (for example, phosphorus (P), arsenic (As), or antimony (Sb)), or a p-type silicon wafer obtained by doping a silicon wafer with a trivalent atom (for example, boron (B) or gallium (Ga)). The silicon of the silicon wafer may be, for example, any of amorphous silicon, single crystal silicon, polycrystalline silicon, or polysilicon.

In addition to those described above, the object to be treated may include various layers and/or structures as desired. For example, the substrate may include a metal wire, a gate electrode, a source electrode, a drain electrode, an insulating layer, a ferromagnetic layer, and/or a non-magnetic layer.

The substrate may include an exposed integrated circuit structure, for example, an interconnect mechanism such as a metal wire or a dielectric material. Examples of the metal and the alloy which are used in the interconnect mechanism include aluminum, a copper-aluminum alloy, copper, titanium, tantalum, cobalt, silicon, titanium nitride, tantalum nitride, and tungsten. The substrate may include layers of silicon oxide, silicon nitride, silicon carbide, and/or silicon oxide dope with carbon.

The manufacturing method for the object to be treated is not particularly limited as long as it is a method that is generally carried out in this field.

Examples of the method of forming the insulating film on a wafer constituting a substrate include a method in which a wafer constituting a substrate is subjected to a heat treatment in the presence of an oxygen gas to form a silicon oxide film, and then a gas of silane and ammonia is introduced thereto to form a silicon nitride film by a chemical vapor deposition (CVD) method.

Examples of the method of forming the metal-containing layer on a wafer constituting a substrate include a method in which a circuit is formed on the wafer having an insulating film by a publicly known method such as resist, and then a metal-containing layer is formed by a method such as plating, a sputtering method, a CVD method, or a molecular beam epitaxy (MBE) method.

The object to be treated may be a substrate that has been subjected to a flattening treatment such as a CMP treatment after providing an insulating film, a barrier metal, and a metal-containing film on the wafer. The CMP treatment is a treatment of flattening a surface of a substrate having a metal-containing film, a barrier metal, and an insulating film by a combined action of chemical action using a polishing slurry containing polishing fine particles (abrasive grains) and mechanical polishing.

The surface of the substrate that has been subjected to the CMP treatment may have impurities remaining thereon, such as abrasive grains (for example, silica and alumina) used in the CMP treatment, a polished metal-containing film, and metal impurities (metal residue) derived from the barrier metal. For example, since these impurities may short-circuit the wiring lines and deteriorate the electrical characteristics of the substrate, the substrate that has been subjected to the CMP treatment is subjected to a washing treatment for removing these impurities from the surface.

Specific examples of the substrate that has been subjected to the CMP treatment include the substrate that has been subjected to a CMP treatment, described in Vol. 84, No. 3, 2018, but the present invention is not limited thereto.

### [Step A]

Examples of the present treatment method include a step A of bringing the present composition into contact with an object to be treated containing a metal-containing substance to remove the metal-containing substance on a substrate.

The composition that is used in the step A is as described above.

In addition, the substrate having a metal-containing substance, which is the object to be treated in the step A, is as described above. The substrate having a metal-containing substance is preferably a substrate having a W-containing substance or a substrate having a Mo-containing substance.

The method of bringing the composition into contact with an object to be treated is not particularly limited, and examples thereof include a method of immersing an object to be treated in the composition contained in a tank, a method of spraying the composition onto a substrate, a method of flowing the composition onto a substrate, and any combination thereof. Among these, a method of immersing, in the composition, a substrate which is an object to be treated and has a metal-containing substance, is preferable.

In order to further enhance the treatment ability of the composition, a mechanical stirring method may also be used.

Examples of the mechanical stirring method include a method of circulating a composition on a substrate, a method of flowing or spraying a composition on a substrate, and a method of stirring a composition with an ultrasonic wave or a megasonic wave.

In addition, the treatment by immersion may be a batch method in which a plurality of objects to be treated are immersed in a treatment tank or may be a single substrate method.

The treatment time in the step A may be adjusted according to the method of bringing the composition into contact with a substrate, the temperature of the composition, and the like. The treatment time (the time for contact between the composition and the object to be treated) is not particularly limited, and is preferably 0.25 to 10 minutes, and more preferably 0.5 to 2 minutes.

The temperature of the composition at the time of treatment is not particularly limited; however, the lower limit thereof is preferably 15° C. or higher, more preferably 20° C. or higher, and still more preferably 30° C. or higher. In addition, the upper limit of the temperature is preferably 80° C. or lower, more preferably 75° C. or lower, and still more preferably 70° C. or lower.

Examples of the specific aspect of the step A include a step A1 of subjecting a wiring line consisting of a metal-containing substance, disposed on a substrate, to a recess-etching treatment by using a composition, a step A2 of removing a film on an outer edge of a substrate on which a film consisting of a metal-containing substance is disposed, by using a composition, a step A3 of removing a metal-containing substance that has adhered to a back surface of a substrate on which a film consisting of a metal-containing substance is disposed, using a composition, a step A4 of removing a metal-containing substance on a substrate after dry etching, by using a composition, and a step A5 of removing a metal-containing substance on a substrate after the chemical-mechanical polishing treatment, by using a composition.

For the steps A1 to A5, the description in paragraphs [0049] to [0072] of WO2019/138814A, the contents of which are incorporated in the present specification by reference, can be referenced.

### [Rinsing Step]

The present treatment method may further include a step of rinsing the object to be treated with a solvent (hereinafter, referred to as a “step B”), after the step A.

The step B is carried out continuously after the step A, and it is preferably a step of carrying out rising with a rinsing solvent (a rinsing liquid) for 5 seconds to 5 minutes. The step B may be carried out using the above-described mechanical stirring method.

Examples of the solvent of the rinsing liquid include deionized (DI) water, methanol, ethanol, isopropanol, N-methylpyrrolidinone, γ-butyrolactone, dimethyl sulfoxide, ethyl lactate, and propylene glycol monomethyl ether acetate.

The solvent of the rinsing liquid is preferably DI water, methanol, ethanol, isopropanol, or a mixed liquid thereof, and more preferably DI water, isopropanol, or a mixed liquid of DI water and isopropanol.

As a method of bringing the rinsing solvent into contact with the object to be treated, the above-described method of bringing the composition into contact with the object to be treated can be similarly applied.

The temperature of the rinsing solvent in the step B is preferably 16° C. to 27° C.

### [Drying Step]

The present treatment method may include a step C of drying the object to be treated, after the step B.

The drying method is not particularly limited, and examples thereof include a spin drying method, a method of flowing a dry gas onto an object to be treated, a method of heating a substrate by a heating means such as a hot plate and an infrared lamp, a Marangoni drying method, a Rotagoni drying method, an isopropanol (IPA) drying method, and any combinations thereof.

The drying time in the step C depends on the drying method; however, it is preferably 20 seconds to 5 minutes.

In the step C, it is preferable to dry a substrate by heating the substrate with a heating means from the viewpoint of excellent removability of the composition in the SiOx layer.

In that case, the heating temperature is not particularly limited; however, it is preferably 50° C. to 350° C. and more preferably more than 100° C. and less than 400° C. from the viewpoint of being more excellent in the balance between removability of the composition in the SiOx layer and the film reduction in the Co film and the SiOx layer, and it is still more preferably 150° C. to 250° C. from the viewpoint of being more excellent in the removability of the composition in the Co film and the SiOx layer.

### [Second Aspect]

Examples of another aspect of the present treatment method include the following second aspect.

That is, it is a treatment method for an object to be treated, which includes a step P of subjecting an object to be treated (a substrate) having a metal-containing layer to an oxidation treatment to oxidize a surface layer of the metal-containing layer, thereby forming a metal oxide layer, and a step Q of bringing the composition into contact with the surface of the metal oxide layer of the object to be treated, where the metal oxide layer has been formed in the step P, to dissolve the metal oxide layer.

The metal-containing layer is preferably, for example, a simple body or alloy of a metal. In addition, the metal-containing layer preferably contains at least one selected from the group consisting of cobalt, copper, tungsten, titanium, and aluminum. That is, the metal-containing layer is preferably a cobalt-containing substance (a cobalt simple body, a cobalt alloy, or the like), a copper-containing substance (a simple body, an alloy, or the like), a tungsten-containing substance (a tungsten simple body, a tungsten alloy, or the like), a titanium-containing substance (a titanium simple body, a titanium alloy, or the like), or an aluminum-containing substance (an aluminum simple body, an aluminum alloy, or the like) is preferable, and a tungsten-containing substance is more preferable.

The metal oxide layer is a layer obtained by oxidizing the surface layer of the metal-containing layer. A part of the surface layer of the metal-containing layer may be a metal oxide layer, or the entire surface layer of the metal-containing layer may be a metal oxide layer.

The metal oxide layer is a layer consisting of a metal simple body or an oxide of an alloy, and it is preferably a layer consisting of a cobalt oxide, an oxide of a cobalt alloy, a copper oxide, an oxide of a copper alloy, a tungsten oxide, an oxide of a tungsten alloy, a titanium oxide, an oxide of a titanium alloy, an aluminum oxide, or an oxide of an aluminum alloy, more preferably a layer consisting of a tungsten oxide or an oxide of a tungsten alloy, and still more preferably a layer consisting of a tungsten oxide.

The thickness of the metal oxide layer is, for example, a thickness equivalent to that of a mono-atomic to deca-atomic layer. It is noted that the thickness of the mono-atomic layer of the metal and the metal oxide is 1 nm or less (for example, 0.3 to 0.4 nm).

In many cases, the metal oxide layer has high solubility (is easily etched) in the composition as compared with the metal-containing layer.

That is, in the second aspect, the surface of the metal-containing layer is made to be a thin metal oxide layer in the step P, and only the above-described metal oxide layer (and a part where the metal-containing layer present in the underlayer of the metal oxide layer may be unavoidably dissolved) is (are) removed in the step Q by using the composition, whereby only a very thin surface of the metal-containing layer contained in the object to be treated can be removed (dissolved).

Further, in a case of repeatedly carrying out the step P and the step Q alternately, it is possible to control the etching amount with high accuracy.

In a case where the step P and the step Q are alternately carried out, the number of times of each step to be carried out may be, for example, 1 to 20 cycles in a case where a combination of the step P and the step Q is defined as one cycle. From the viewpoint of excellent control of the recess amount, the number of times of each step to be carried out in a case where the step P and the step Q are alternately carried out is preferably 3 cycles or more and more preferably 5 cycles or more.

The object to be treated to which the second aspect can be applied may include one kind of metal-containing layer alone or may include two or more kinds thereof. In addition, the object to be treated to which the second aspect can be applied may contain a metal-based material other than the metal-containing layer or the metal oxide layer, and a part or whole of such metal-based material may be removed intentionally or unavoidably through the step P and the step Q.

The step P is a step of subjecting the object to be treated including the metal-containing layer to an oxidation treatment to oxidize the surface layer of the metal-containing layer, thereby forming a metal oxide layer.

There is no limitation on the method of the oxidation treatment for oxidizing the surface layer of the metal-containing layer to form a metal oxide layer, and the method can be carried out by subjecting the object to be treated to a liquid treatment in which the object to be treated is brought into contact with an oxidizing liquid, a gas treatment in which an oxidation gas is brought into contact (an ozone treatment in which an ozone gas is brought into contact with a substrate described later, a heating treatment in oxygen in which heating is carried out in an oxygen atmosphere, or the like), or a plasma treatment using an oxygen gas.

One kind of oxidation treatment may be carried out alone, or two or more kinds thereof may be carried out in combination.

Among the above, as the oxidation treatment, it is preferable to at least carry out a liquid treatment in which a predetermined oxidizing liquid is brought into contact with the object to be treated.

The oxidizing liquid may be any chemical liquid that can oxidize the surface layer of the metal-containing layer. The oxidizing liquid is preferably an oxidizing liquid other than the present composition.

The oxidizing liquid is preferably a chemical liquid (hereinafter, also referred to as a “specific chemical liquid”) selected from the group consisting of water, hydrogen peroxide water, a mixed aqueous solution of ammonia and hydrogen peroxide (APM), a mixed aqueous solution of hydrofluoric acid and hydrogen peroxide water (FPM), a mixed aqueous solution of sulfuric acid and hydrogen peroxide water (SPM), a mixed aqueous solution of hydrochloric acid and hydrogen peroxide water (HPM), oxygen-dissolved water, ozone-dissolved water, perchloric acid, and nitric acid.

Regarding the composition of the hydrogen peroxide water, for example, the content of H₂O₂ is 0.5% to 31% by mass and more preferably 3% to 15% by mass with respect to the total mass of the hydrogen peroxide water.

The composition of APM is, for example, preferably within a range (in terms of mass ratio) of “ammonia water:hydrogen peroxide water:water=1:1:1” to “ammonia water:hydrogen peroxide water:water=1:3:45”.

The composition of FPM is, for example, preferably within a range (in terms of mass ratio) of “hydrofluoric acid: hydrogen peroxide water:water=1:1:1” to “hydrofluoric acid: hydrogen peroxide water: water=1:1:200”.

The composition of SPM is, for example, preferably within a range (in terms of mass ratio) of “sulfuric acid:hydrogen peroxide water:water=3:1:0” to “sulfuric acid:hydrogen peroxide water:water=1:1:10”.

The composition of HPM is, for example, preferably within a range (in terms of mass ratio) of “hydrochloric acid:hydrogen peroxide water:water=1:1:1” to “hydrochloric acid:hydrogen peroxide water:water=1:1:30”.

It is noted that these descriptions of the preferred compositional ratios are intended to be compositional ratios in a case where the ammonia water is 28% by mass ammonia water, the hydrofluoric acid is 49% by mass hydrofluoric acid, the sulfuric acid is 98% by mass sulfuric acid, the hydrochloric acid is 37% by mass hydrochloric acid, and the hydrogen peroxide water is 30% by mass hydrogen peroxide water.

The volume ratio is based on the volume at room temperature.

The description [“A:B:C=x:y:z” to “A:B:C=X:Y:Z” ] as a suitable range indicates that it is preferable to satisfy at least one range of [“A:B=x:y” to “A:B=X:Y” ], [“B:C=y:z” to “B:C=Y:Z” ], or [“A:C=x:z” to “A:C=X:Z” ] (preferably two ranges and more preferably all the ranges).

The composition of the oxygen-dissolved water is, for example, that of an aqueous solution in which the content of O₂ is 20 to 500 ppm by mass with respect to the total mass of the oxygen-dissolved water.

The composition of the ozone-dissolved water is, for example, that of an aqueous solution in which the content of O₃ is 1 to 60 ppm by mass with respect to the total mass of the ozone-dissolved water.

The perchloric acid is, for example, an aqueous solution in which the content of HClO₄ is 0.001% to 60% by mass with respect to the total mass of the solution.

The nitric acid is, for example, an aqueous solution in which the content of HNO₃ is 0.001% to 60% by mass with respect to the total mass of the solution.

In the liquid treatment, a method of bringing an object to be treated into contact with the oxidizing liquid (preferably the specific chemical liquid) is not particularly limited, and examples thereof include a method of immersing an object to be treated, in the oxidizing liquid charged in a tank, a method of spraying the oxidizing liquid onto an object to be treated, a method of allowing the oxidizing liquid to flow onto an object to be treated, and any combination thereof.

The time for contact between the object to be treated and the oxidizing liquid is, for example, preferably 0.25 to 10 minutes and more preferably 0.5 to 5 minutes.

The temperature of the oxidizing liquid is preferably 20° C. to 75° C. and more preferably 20° C. to 60° C.

In the gas treatment, examples of the oxidation gas that comes into contact with an object to be treated include dry air, an oxygen gas, an ozone gas, and a mixed gas thereof. The oxidation gas may contain a gas other than the above-described gas.

In the gas treatment, the oxidation gas that comes into contact with an object to be treated is preferably an oxygen gas or an ozone gas. In a case of being brought into contact with an object to be treated, an oxygen gas or an ozone gas is preferably brought into contact under an oxygen atmosphere, an ozone atmosphere, or a mixed gas atmosphere of oxygen and ozone.

In the gas treatment, an aspect in which an object to be treated is heated (for example, heating at 40° C. to 200° C.) while being brought into contact with the oxidation gas is also preferable.

Among them, the gas treatment is preferably an ozone treatment in which an ozone gas is brought into contact with an object to be treated or a heating treatment in oxygen in which an object to be treated is heated in an oxygen atmosphere.

In the ozone treatment, an ozone gas may be brought into contact with an object to be treated in an ozone atmosphere, or ozone gas may be brought into contact with an object to be treated in an atmosphere of a mixed gas of an ozone gas and another gas (for example, an oxygen gas). In addition, the ozone treatment may be a treatment in which an object to be treated is heated while being brought into contact with an ozone gas.

In the step P (particularly in the case of carrying out a liquid treatment), the object to be treated may contain a metal-based material other than the metal-containing layer of which the surface layer is oxidized by the oxidation treatment to be a metal oxide layer, and a part or whole of such a metal-based material may be intentionally or unavoidably removed by the step P (particularly, the liquid treatment).

In addition, in the step P (particularly in the case of carrying out the liquid treatment), a part of the metal-containing layer of the object to be treated may be intentionally or unavoidably removed.

The step Q is a step of bringing the object to be treated obtained in the step P into contact with the composition to dissolve the metal oxide layer.

In the step Q, the method of bringing the object to be treated into contact with the composition is not particularly limited, and examples thereof include the same method as the method of bringing the object to be treated into contact with the oxidizing liquid.

The time for contact between the object to be treated and the composition is, for example, preferably 0.25 to 10 minutes and more preferably 0.5 to 5 minutes.

The temperature of the composition is preferably 20° C. to 75° C. and more preferably 20° C. to 60° C.

In the step Q, the removal of the metal oxide layer may be carried out partially or entirely.

In the step Q, a part or whole of the metal-containing layer (for example, the metal-containing layer that has been exposed to the surface layer by removing the metal oxide layer that covers the surface) may be intentionally or unavoidably removed.

In the step Q, the object to be treated may contain another metal-based material other than the metal oxide layer and the metal-containing layer that forms the metal oxide layer on the surface, and a part or whole of such a metal-based material may be intentionally or unavoidably removed.

It is noted that in a case where the metal-containing layer and/or the other metal-based material is not intentionally dissolved, it is preferable that the amount of the metal-containing layer and/or the other metal-based material, which is unavoidably dissolved, is small.

The composition to be subjected to the step Q may be subjected to a degassing treatment or the like in advance to reduce the amount of dissolved oxygen. The metal-containing layer exposed by removing the metal oxide layer with the composition is oxidized by the dissolved oxygen in the composition to become a new metal oxide layer, and such a metal oxide layer is further removed by the composition, and as a result, it is possible to suppress the removal of an excessive amount of the metal-containing layer by reducing the amount of dissolved oxygen in the chemical liquid.

The present treatment method may be carried out in combination before or after other steps which are carried out in the manufacturing method for a semiconductor device. Another step may be incorporated into the execution of the present treatment method, or the present treatment method may be incorporated into the other step.

Examples of the other step include a step of forming each structure such as a metal wire, a gate structure, a source structure, a drain structure, an insulating layer, a ferromagnetic layer, and/or a non-magnetic layer (layer formation, etching, chemical mechanical polishing, modification, or the like), a resist forming step, an exposure step and a removal step, a heat treatment step, a washing step, and an examination step.

The present treatment method may be carried out at any stage of a back-end-of-the-line (BEOL) process, a middle-of-the-line (MOL) process, and a front-end-of-the-line (FEOL) process.

### EXAMPLES

Hereinbelow, the present invention will be described in more detail with reference to Examples. The material, the using amount, the proportion, and the like shown in the Examples below may be modified as appropriate as long as the modifications do not depart from the spirit of the present invention. That is, the scope of the present invention should not be construed as being limited to Examples shown below.

In the following Examples, the pH of the composition was measured at 25° C. using a pH meter (manufactured by HORIBA, Ltd., model “F-74”) in accordance with JIS Z8802-1984.

In addition, in the production of compositions of Examples and Comparative Examples, handling of the container, and preparation, filling, storage, and analytical measurement of the compositions were all carried out in a clean room satisfying a level of ISO Class 2 or lower.

### [Raw Material of Composition]

The following compounds were used to prepare the composition. It is noted that as the various components used in Example, those all classified into a semiconductor grade or a high-purity grade equivalent thereto were used.

### <Specific Resin>

Resins E-1 to E-24 and CE-1 shown below were used as the specific resin.

In the following structural formulae, “Mw” is a weight-average molecular weight of each resin measured by the method described above. In the structural formulae of the resins other than the resins E-7 and CE-1, the numbers appended to the repeating units represent the compositional ratios (molar fractions) of the respective repeating units in the resin. In the structural formulae of the resins E-1 to E-6, E-8 to E-19, and E-22 to E-24, the repeating unit A is described on the left side of the paper surface, and the repeating unit B is described on the right side of the paper surface. In addition, among three repeating units described in the structural formulae of the resins E-20 and E-21, the repeating unit on the left side corresponds to the repeating unit A, and the two repeating units in the center and on the right side correspond to the repeating unit B. It is noted that the compositional ratio of each repeating unit in the resin was measured by ¹³C-NMR.

As a result of measuring the solubility of each of the resins E-1 to E-24 and the resin CE-1 in water at 25° C., according to the integrating sphere type photoelectric photometry according to JIS K 0101 (2017) using a turbidity meter (“PT-200” manufactured by Mitsubishi Chemical Analytech Co., Ltd.), it was 0.01% to 30.0% by mass.

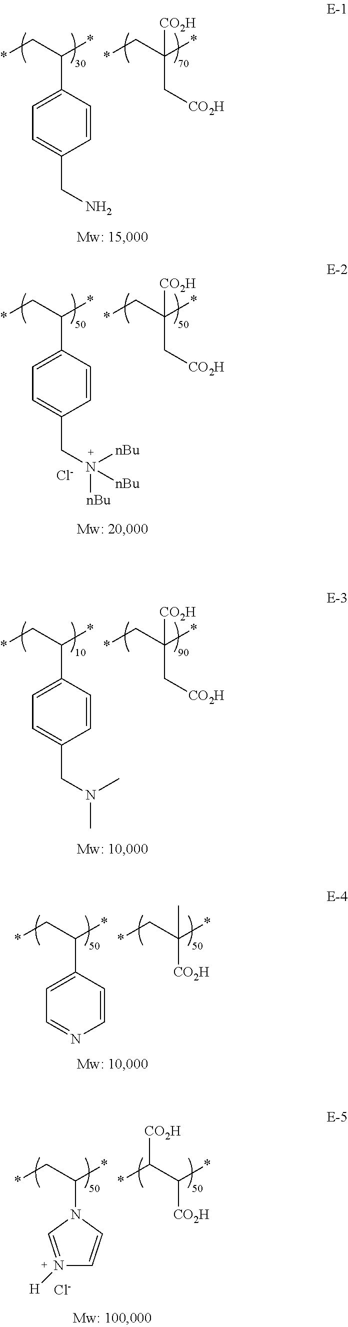

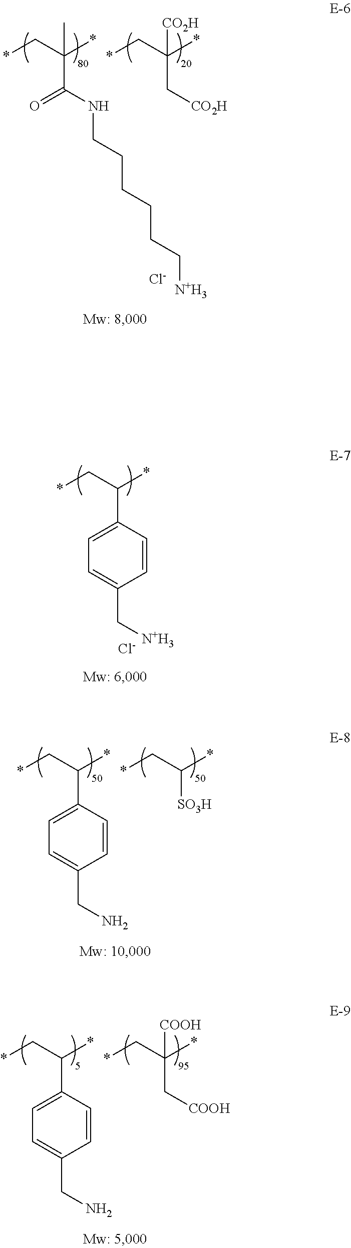

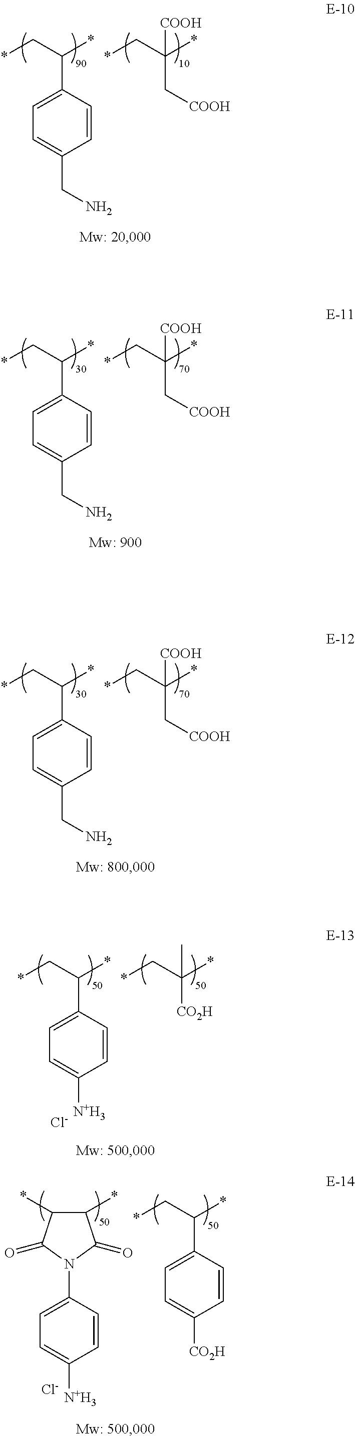

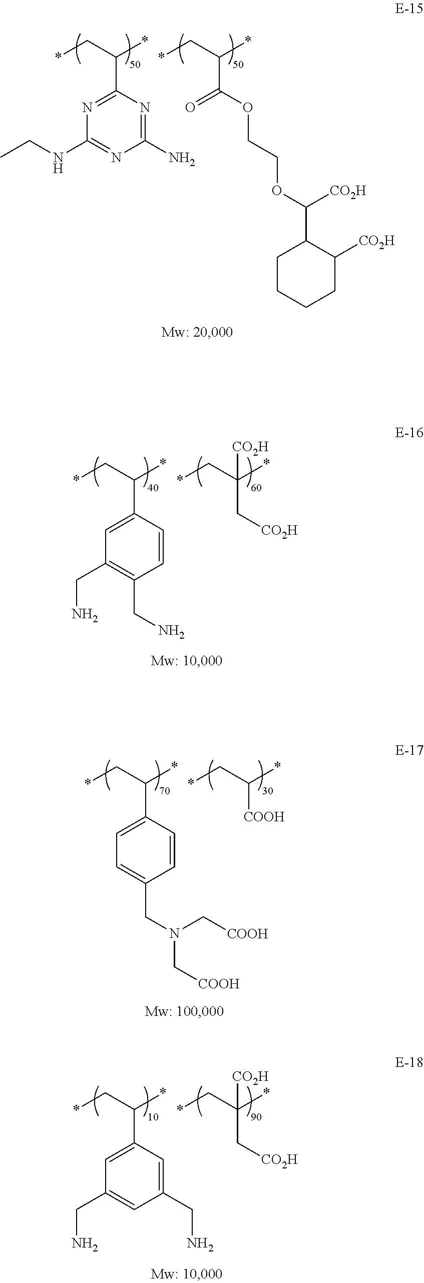

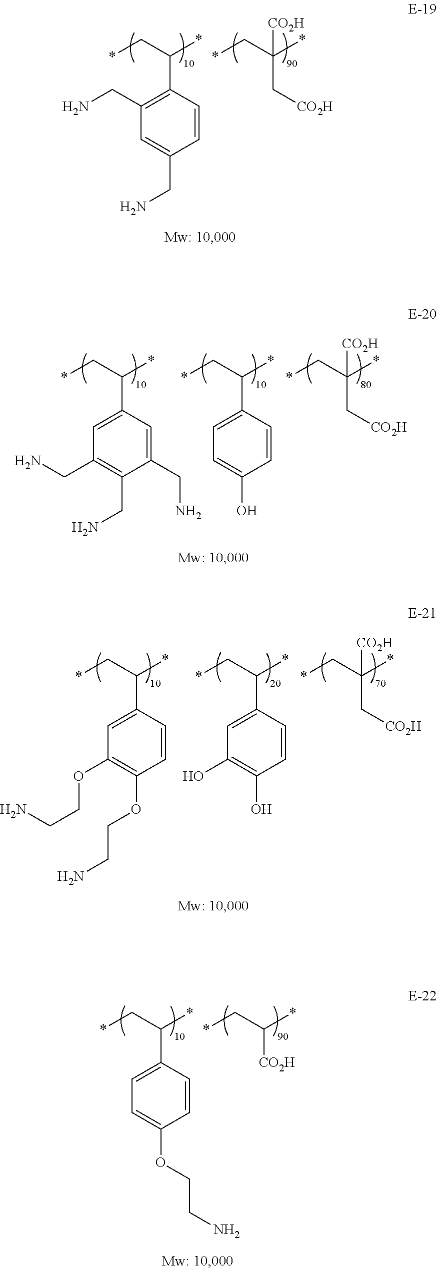

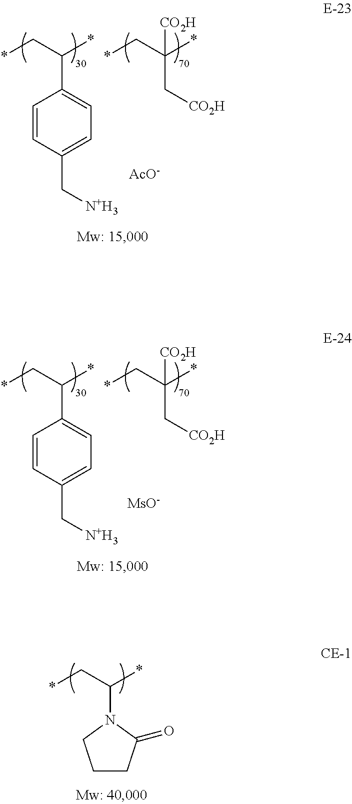

Hereinafter, the synthesis method for the resin E-16 will be shown in Synthesis Example 1, and the synthesis method for the resin E-18 will be shown in Synthesis Example 2.

### (Synthesis Example 1: Synthesis of Resin E-16)

An intermediate E-16A, an intermediate E-16B, an intermediate E-16C, an intermediate E-16D, and a compound E-16E which is a polymerizable compound A were sequentially synthesized according to a synthesis procedure shown in the following scheme, and then the compound E-16E was copolymerized with a maleic acid which is a polymerizable compound B to synthesize the resin E-16 as a specific resin.

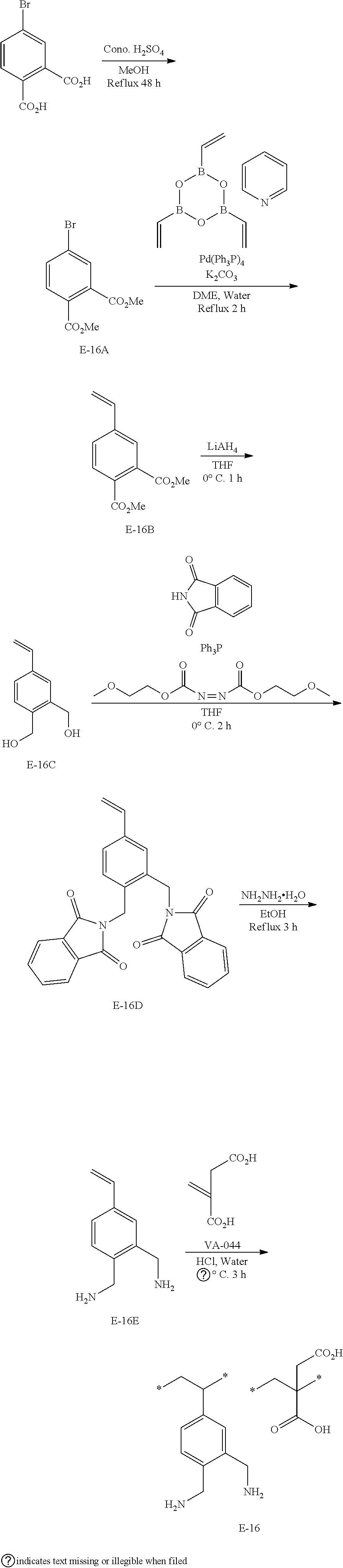

Hereinafter, each synthesis procedure will be described in detail.

### Synthesis Example 1-1: Synthesis of Intermediate E-16A

4-Bromophthalic acid (22 g, 89.8 mmol, manufactured by Tokyo Chemical Industry Co., Ltd.) and methanol (220 mL, manufactured by FUJIFILM Wako Pure Chemical Corporation) were added to a 0.5 L three-neck flask under a nitrogen flow (50 mL/min), and the obtained mixed liquid was stirred at 25° C. Concentrated sulfuric acid (0.22 g, 2.24 mmol, manufactured by Kanto Chemical Co., Inc.) was further added to the three-neck flask, and the mixed liquid was heated and refluxed for 72 hours while distilling off water from the mixed liquid. Then, methanol was distilled off from the reaction solution under a reduced pressure of 40° C./10 hPa.

After the distillation, ethyl acetate (200 mL, manufactured by FUJIFILM Wako Pure Chemical Corporation) and a saturated aqueous sodium hydrogen carbonate solution (200 mL) were added to the concentrated solution, and the mixed liquid was transferred to a 1 L separatory funnel, and then stirring was carried out. After stopping the stirring, the lower layer (water layer) of the two separated layers was removed, and a saturated aqueous sodium hydrogen carbonate solution (200 mL) was added to the remaining upper layer (organic layer), followed by stirring. After stopping the stirring, the lower layer (water layer) of the two separated layers was removed, and distilled water (200 mL) was added to the remaining upper layer (organic layer), followed by stirring. After stopping the stirring, the lower layer (water layer) of the two separated layers was removed, and the remaining upper layer (organic layer) was transferred to a 0.5 L eggplant flask. Next, the solvent was distilled off from the organic layer under a reduced pressure of 40° C./10 hPa to obtain an intermediate E-16A as a target substance.

### Synthesis Example 1-2: Synthesis of Intermediate E-16B

the intermediate E-16A (22 g, 80.6 mmol), dimethoxyethane (DME) (220 mL, manufactured by FUJIFILM Wako Pure Chemical Corporation), and distilled water (66 mL) were added to a 0.5 L three-neck flask under a nitrogen flow (50 mL/min), and the obtained mixed liquid was stirred at 25° C. 2,4,6-trivinylboroxine-pyridine complex (19.4 g, 80.6 mmol, manufactured by Tokyo Chemical Industry Co., Ltd.), tetrakis(triphenylphosphine)palladium (0) (0.93 g, 0.8 mmol, manufactured by Tokyo Chemical Industry Co., Ltd.), and potassium carbonate (0.93 g, 0.8 mmol, manufactured by Tokyo Chemical Industry Co., Ltd.) were further added to the three-neck flask in this order, and the mixed liquid was heated and refluxed for 3 hours. After cooling the reaction solution to room temperature, the reaction solution was filtered using a filter obtained by spreading celite on a Buechner funnel. The obtained filtrate was transferred to a 0.5 L eggplant flask, and the solvent was distilled off from the filtrate under a reduced pressure of 40° C./10 hPa.

After the distillation, ethyl acetate (200 mL, manufactured by FUJIFILM Wako Pure Chemical Corporation) and a saturated saline (200 mL) were added to the concentrated solution, and the mixed liquid was transferred to a 1 L separatory funnel, and then stirring was carried out.

After stopping the stirring, the lower layer (water layer) of the two separated layers was removed, and 1 M hydrochloric acid (200 mL) was added to the remaining upper layer (organic layer), followed by stirring. After stopping the stirring, the lower layer (water layer) of the two separated layers was removed, and 1 M hydrochloric acid (200 mL) was added to the remaining upper layer (organic layer), followed by stirring. After stopping the stirring, the lower layer (water layer) of the two separated layers was removed, and saturated saline (200 mL) was added to the remaining upper layer (organic layer), followed by stirring. After stopping the stirring, the lower layer (water layer) of the two separated layers was removed, and saturated saline (200 mL) was added to the remaining upper layer (organic layer), followed by stirring. After stopping the stirring, the lower layer (water layer) of the two separated layers was removed, and saturated saline (200 mL) was added to the remaining upper layer (organic layer), followed by stirring. After stopping the stirring, the lower layer (water layer) of the two separated layers was removed, and the remaining upper layer (organic layer) was transferred to a 0.5 L eggplant flask to distill off the solvent from the organic layer under a reduced pressure of 40° C./10 hPa. The obtained crude product was purified by silica gel chromatography to obtain an intermediate E-16B as a target substance.

### Synthesis Example 1-3: Synthesis of Intermediate E-16C

The intermediate E-16B (14 g, 49.8 mmol) and tetrahydrofuran (THF) (181 mL, manufactured by FUJIFILM Wako Pure Chemical Corporation) were added to a 0.5 L three-neck flask under a nitrogen flow (50 mL/min), and the mixed liquid was cooled to 0° C. Lithium aluminum hydride (LiAlH₄) (3.97 g, 104.6 mmol, manufactured by Tokyo Chemical Industry Co., Ltd.) was added to the three-neck flask little by little over 15 minutes, and the mixed liquid was stirred for 1 hour. Then, distilled water (4 mL), a 15% sodium hydroxide aqueous solution (4 mL), and distilled water (12 mL) were added in this order to the mixed liquid, and the mixed liquid was stirred at 25° C. for 1 hour. Thereafter, the reaction solution was filtered by using a filter formed by spreading celite on a Buechner funnel. The obtained filtrate was transferred to a 0.5 L eggplant flask, and the solvent was distilled off from the filtrate under a reduced pressure of 40° C./10 hPa to obtain an intermediate E-16C as a target substance.

The structure of the obtained intermediate E-16C was identified by ¹H-NMR.

¹H-NMR (400 MHz, DMSO-d6): δ=7.45 (s, 1H), 7.30-7.39 (m, 2H), 6.72 (dd, J=10.9 Hz, 17.7 Hz, 1H), 5.78 (d, J=17.6 Hz, 1H), 5.21 (d, J=10.9 Hz, 1H), 5.02-5.13 (m, 2H), 4.52 (t, J=4.9 Hz, 4H).

### Synthesis Example 1-4: Synthesis of Intermediate E-16D

The intermediate E-16C (4.8 g, 29.3 mmol), triphenylphosphine (Ph₃P) (20.0 g, 76.2 mmol, manufactured by FUJIFILM Wako Pure Chemical Corporation), phthalimide (11.2 g, 76.2 mmol, manufactured by FUJIFILM Wako Pure Chemical Corporation), and tetrahydrofuran (60 mL, manufactured by FUJIFILM Wako Pure Chemical Corporation) were added to a 0.3 L three-neck flask under a nitrogen flow (50 mL/min), and the mixed liquid was cooled to 0° C. Separately, a solution of bis(2-methoxyethyl) azodicarboxylate (17.8 g, 76.2 mmol, manufactured by FUJIFILM Wako Pure Chemical Corporation) and tetrahydrofuran (60 mL) was prepared in a 0.1 L eggplant flask. The obtained solution was added dropwise to the above-d three-neck flask over 2 hours while keeping the internal temperature of the above-described three-neck flask at 5° C. or lower. After completion of the dropwise addition, the reaction solution was stirred at 0° C. for 3 hours, and the precipitate was filtered. The substance obtained by filtration was washed with 60 mL of tetrahydrofuran at 0° C. Then, the substance obtained by filtration was subjected to blast drying at 40° C. for 1 day to obtain an intermediate E-16D as a target substance.

### Synthesis Example 1-5: Synthesis of Compound E-16E

the intermediate E-16D (9.6 g, 23.3 mmol) and ethanol (197 mL, manufactured by FUJIFILM Wako Pure Chemical Corporation) were added to a 0.3 L three-neck flask under a nitrogen flow (50 mL/min), and the mixed liquid was stirred. Hydrazine monohydrate (7.0 g, 140.0 mmol, manufactured by FUJIFILM Wako Pure Chemical Corporation) was further added to the three-neck flask, and the mixed liquid was heated and refluxed for 5 hours. Thereafter, the reaction solution was cooled to 0° C., and the precipitate was filtered. The substance obtained by filtration was washed with 60 mL of tetrahydrofuran at 0° C. Next, the filtrate was concentrated, dichloromethane (300 mL, manufactured by FUJIFILM Wako Pure Chemical Corporation) and a 20% by mass sodium hydroxide aqueous solution (300 mL) were added to the concentrated solution, the mixed liquid was transferred to a 1 L separatory funnel, and stirring was carried out. After allowing the mixed liquid to stand, the mixed liquid was concentrated to obtain a compound E-16E, which is a target substance and the polymerizable compound A.

The structure of the obtained compound E-16E was identified by ¹H-NMR.

¹H-NMR (400 MHz, DMSO-d6): δ=7.45 (s, 1H), 7.24-7.35 (m, 2H), 6.70 (dd, J=10.9 Hz, 17.7 Hz, 1H), 5.79 (d, J=17.6 Hz, 1H), 5.20 (d, J=10.9 Hz, 1H), 3.67-3.78 (m, 4H), 1.51-1.89 (m, 4H).

### Synthesis Example 1-6: Synthesis of Resin E-16

The compound E-16E (0.85 g, 5.2 mmol) and distilled water (2.54 g) were added to a 0.05 L three-neck flask under a nitrogen flow (50 mL/min), and the mixed liquid was cooled to 0° C. A 1 M hydrochloric acid aqueous solution (10.4 mL) and itaconic acid (0.68 g, 5.2 mmol, manufactured by FUJIFILM Wako Pure Chemical Corporation) were further added to the three-neck flask, and the mixed liquid was stirred at 70° C. for 30 minutes. Separately, a water-soluble azo polymerization initiator “VA-044” (0.17 g, 0.5 mmol, manufactured by FUJIFILM Wako Pure Chemical Corporation) and distilled water (0.85 mL) were added to a 5 mL test tube to prepare a solution of the azo polymerization initiator. The obtained solution was added to the above-described three-neck flask, and the mixed liquid was stirred at 70° C. for 3 hours. After confirming that all the monomers were consumed by using ¹H-NMR, the reaction solution was filtered to obtain an aqueous solution of the resin E-16, which is a target substance and a specific resin (concentration of solid contents: 13.1%).

The structure of the obtained resin E-16 was identified by ¹H-NMR. [0275] ¹H-NMR (400 MHz, D₂O): δ=6.60-8.50 (broad, 3H), 3.90-4.35 (broad, 4H), 0.70-2.90 (broad, 7H).

### Synthesis Example 2: Synthesis of Resin E-18

An intermediate E-18A, an intermediate E-18B, an intermediate E-18C, and an intermediate E-18D which is a polymerizable compound A were sequentially synthesized according to a synthesis procedure shown in the following scheme, and then the compound E-18D was copolymerized with a maleic acid which is a polymerizable compound B to synthesize the resin E-18 as a specific resin.

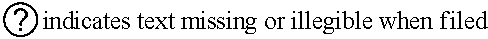

### Synthesis Example 2-1: Synthesis of Compound E-18D

A compound E-18D, which is a target substance and the polymerizable compound A, was synthesized according to the same procedure as in Synthesis Examples 1-2 to 1-5, except that in Synthesis Example 1-2, dimethyl 5-Bromoisophthalate (manufactured by FUJIFILM Wako Pure Chemical Corporation) was used instead of the intermediate E-16A.

The structure of the obtained compound E-18D was identified by ¹H-NMR.

¹H-NMR (400 MHz, DMSO-d6): δ=7.26 (s, 2H), 7.18 (s, 1H), 6.70 (dd, J=10.9 Hz, 17.7 Hz, 1H), 5.80 (dd, J=1.0 Hz, 17.6 Hz, 1H), 5.20 (dd, J=0.9 Hz, 10.9 Hz, 1H), 3.68 (s, 4H), 1.73 (broad, 4H).

### Synthesis Example 2-2: Synthesis of Resin E-18

The resin E-18, which is a target substance and a specific resin, was synthesized according to the same procedure as in Synthesis Example 1-6, except that the compound E-18D synthesized as a raw material compound was used instead of the compound E-16E.

The structure of the obtained resin E-18 was identified by ¹H-NMR.

¹H-NMR (400 MHz, D₂O): δ=7.58-8.75 (broad, 1H), 6.65-7.58 (broad, 2H), 3.65-4.22 (broad, 4H), 0.35-2.80 (broad, 7H).

Each of resins E-1 to E-15, E-17, and E-19 to E-24, which are specific resins having the above-described repeating unit A, was synthesized according to the same scheme and the same procedure as in Synthesis Example 1 or 2, except that the raw material compound was appropriately changed.

### <Removing Agent>

Hydroxylamine (corresponding to a hydroxylamine compound)
Tetramethylammonium hydroxide (TMAH) (corresponding to a basic compound (quaternary ammonium compound))
Ammonium hydroxide (corresponding to a basic compound)
Hydrofluoric acid (corresponding to a fluorine-containing compound)
Sulfuric acid (corresponding to acidic compound (inorganic acid))
Citric acid (corresponding to an acidic compound (organic acid))
Tartaric acid (corresponding to an acidic compound (organic acid))
Succinic acid (corresponding to an acidic compound (organic acid))
Ethylenediaminetetraacetic acid (EDTA) (corresponding to an acidic compound (organic acid))
Monoethanolamine (MEA) (corresponding to a basic compound (water-soluble amine))
Trishydroxymethylaminomethane (Tris) (corresponding to a basic compound (water-soluble amine))
Tris(hydroxyethyl)methylammonium hydroxide (corresponding to a basic compound (quaternary ammonium compound))
2-(Dimethylamino)-2-methyl-1-propanol (corresponding to a basic compound (water-soluble amine))

### <Oxidizing Agent>

Hydrogen peroxide
Nitric acid

### <Corrosion Inhibitor>

5-Methyl-1H-benzotriazole (corresponding to a triazole compound)
5-Mercapto-1-phenyl-1H-tetrazole (corresponding to a tetrazole compound)
n-dodecyl mercaptan (corresponding to a thiol compound)

### <Surfactant>

PERSOFT (registered trademark) SF-T (an alkyl sulfate ester triethanolamine salt, an anionic surfactant, manufactured by NOF CORPORATION)
NISSAN CATION (registered trademark) BB (dodecyltrimethylammonium chloride, a cationic surfactant, manufactured by NOF CORPORATION)
NONION K-220 (polyoxyethylene lauryl ether, a nonionic surfactant, manufactured by NOF CORPORATION)
NISSAN ANON (registered trademark) BL (betaine lauryldimethylaminoacetate, an amphoteric surfactant, manufactured by NOF CORPORATION)

### <Antifoaming Agent>

KF-6701 (a silicone-based antifoaming agent, manufactured by Shin-Etsu Chemical Co., Ltd.)

### <Solvent>

Propylene glycol
Water (ultrapure water)

### <pH Adjusting Agent>

Potassium hydroxide

### Example A

[Preparation of composition]

A preparation method for each of the compositions of Examples A1 to A25 and Comparative Example A1 will be described with reference to Example A1.

Hydroxylamine, 5-methyl-1H-benzotriazole, PERSOFT SF-T, and ultrapure water were mixed such that amounts thereof were the contents shown in Table 4 described below, whereby a mixed liquid C-1 was obtained. Thereafter, the mixed liquid C-1, the resin E-1, and citric acid and monoethanolamine as pH adjusting agents were sufficiently stirred using a stirrer to prepare a composition of Example A1. Tables 1 to 3 show the contents of the resin E-1 and each pH adjusting agent in the obtained composition. In the obtained composition, a remainder other than the resin E-1 and the pH adjusting agent is the mixed liquid C-1.

According to the preparation method for the composition of Example A1, each of compositions of Examples A2 to A25 and Comparative Example A1, which had the compositions shown in Tables 1 to 4, was prepared.

The pH of each of the compositions of Examples A1 to A25 and Comparative Example A1 prepared at 25° C. are shown in Tables 1 to 3.

[Evaluation of composition]

The following test was carried out using the prepared compositions of Examples or Comparative Examples.

### <Solubility>

Each of substrates (Si) on which a W film consisting of a tungsten simple body (W) and a TiAlC film consisting of titanium-aluminum carbide (TiAlC) were respectively laminated at a film thickness of 100 nm was produced, and each of these substrates was cut into a square shape of 2 cm×2 cm to prepare a specimen.

Each of the obtained specimens was immersed in each composition (liquid temperature: 60° C.) for 10 minutes.

Before and after the above-described immersion test, the film thickness of each film was measured with a fluorescent X-ray analysis device (XRF AZX-400, manufactured by Rigaku Corporation) for thin film evaluation. From the measured film thicknesses before and after immersion, the dissolution rate (A/min) of each film in a case where each composition was used was calculated. From the calculated dissolution rate, the solubility for the W film and the TiAlC film was evaluated based on the following evaluation standard.

The evaluation results of the solubility for the obtained W film and TiAlC film are shown in Tables 1 to 3. As the dissolution rate is slower, the dissolution of each film is further suppressed, and “A” indicates that the evaluation of the solubility is highest.

### (Evaluation Standard for Solubility of W Film)

A: The dissolution rate of the W film is 0.5 Å/min or less.

B: The dissolution rate of the W film is more than 0.5 Å/min and 1.0 Å/min or less.

C: the dissolution rate of the W film is 1.0 Å/min or more and 3.0 Å/min or less.

D: The dissolution rate of the W film is more than 3.0 Å/min.

### (Evaluation Standard for Solubility of TiAlC Film)

A: The dissolution rate of the TiAlC film is 0.1 Å/min or less.

B: The dissolution rate of the TiAlC film is more than 0.1 Å/min and 0.5 Å/min or less.

C: The dissolution rate of the TiAlC film is more than 0.5 Å/min and 1.5 Å/min or less.

D: The dissolution rate of the TiAlC film is more than 1.5 Å/min.

### <Residue Removability>

A laminate (corresponding to a laminate before treatment) including a W film, a SiO₂ film, and a metal hard mask (TiN) having a predetermined opening portion, in this order, was formed on a substrate (Si). Using the obtained laminate, dry etching was carried out using the metal hard mask as a mask, and the SiO₂ film was etched until the surface of the W film was exposed to form holes, thereby preparing a sample 1 (FIG. 1). As a result of checking a cross section of this laminate with a photographic image from a scanning electron microscope (SEM), dry etching residues were observed on the wall surface in the holes.

The removability (residue removability) of the dry etching residues was evaluated according to the following procedure.

First, a section (having a square shape of about 2.0 cm×2.0 cm) of the prepared sample 1 was immersed in each composition of which the temperature was adjusted to 60° C. Immediately after 5 minutes had elapsed from the start of the immersion, the section of the sample 1 was taken out, immediately washed with ultrapure water, and dried with N₂. Then, the surface of the section of the immersed sample 1 was observed with SEM to check the presence or absence of the dry etching residues. Similarly, the section of the sample 1, which had been immersed for 10 minutes, was washed with ultrapure water and dried with N₂, and then the surface of the section was observed with SEM to check the presence or absence of the dry etching residues.

The residue removability was evaluated according to the following determination standard from the observation results of each of sections of the sample 1.

### (Evaluation Standard for Residue Removability)

A: The dry etching residues have been completely removed by immersion for 5 minutes.

B: The dry etching residues have not been completely removed by immersion for 5 minutes but have been completely removed by immersion for 10 minutes.

C: The dry etching residues have not been completely removed by immersion for 10 minutes.

<Evaluation of Residual Properties of Composition after Rinsing Treatment>

A substrate (Si) including a SiO₂ film was produced, and the substrate (Si) including the SiO₂ film was subjected to a treatment of immersing it for 10 minutes in each composition adjusted to 60° C. Next, the substrate subjected to the immersion treatment was immersed in a rinsing liquid consisting of isopropanol for 0.5 minutes to carry out a rinsing treatment of the SiO₂ film.

The surface of the SiO₂ film subjected to the rinsing treatment was analyzed by the X-ray photoelectron spectroscopic analysis, and the ratio (unit:atom %) of the number of nitrogen atoms derived from each composition to the number of all atoms on the surface of the SiO₂ film was measured.

The measurement conditions of the X-ray photoelectron spectroscopic analysis are shown below.

### (Measurement Conditions)

Device: Quantera SXMTM manufactured by ULVAC-PHI. Inc.
X-ray source: Monochromatic A1 Kα ray
X-ray beam diameter: φ200 μm
Signal capture angle: 45°

From the measurement results of the number ratio of nitrogen atoms to all atoms on the surface of the SiO₂ film, the residual properties (residual properties after rinsing) of the composition after the rinsing treatment were evaluated.

The obtained evaluation results are shown in Tables 1 to 3. It is preferable that the number ratio of nitrogen atoms to all atoms is smaller. The smaller the number ratio of nitrogen atoms to all atoms is, the higher the solubility of each component of the composition in the rinsing liquid is, which means the residual amount of the composition on the surface of the SiO₂ film after the rinsing treatment is small.

(Evaluation Standard for Residual Properties after Rinsing)

A: The number ratio of nitrogen atoms to all atoms on the surface of the SiO₂ film is 0.1 atom % or less.

B: The number ratio of nitrogen atoms to all atoms on the surface of the SiO₂ film is more than 0.1 atom % and 1.0 atom % or less.

C: The number ratio of nitrogen atoms to all atoms on the surface of the SiO₂ film is more than 1.0 atom % and 3.0 atom % or less.

D: The number ratio of nitrogen atoms to all atoms on the surface of the SiO₂ film is more than 3.0 atom %.

Tables 1 to 4 show the composition of each of the compositions used in Examples A1 to A25 and Comparative Example A1, and Tables 1 to 3 show the evaluation results of Examples A1 to A25 and Comparative Example A1. Table 4 shows the prescription of components of the mixed liquids C-1 to C-6 used for preparing each of the compositions of Examples A1 to A25 and Comparative Example A1.

Tables 1 to 3 show the content of the resin contained in each composition and the total content of all components other than the resin (the contents are in terms of mass), and Table 4 shows the kind and content of components other than the resin, the components being contained in each composition (the contents are in terms of mass).

In the table, the column of “ClogP” of “Resin” indicates the ClogP value of the polymerizable compound A from which the repeating unit A contained in each resin is derived. However, it indicates a ClogP value of vinylpyrrolidone regarding the resin CE-1. The ClogP value was calculated according to the above-described method.

The column of “Ratio a/b” of “Resin” indicates a ratio a/b of a molar number a of the repeating unit A to a molar number b of the repeating unit B in the specific resin having the repeating unit A and the repeating unit B. The repeating unit A and the repeating unit B, which are contained in each resin, are as described above.

In the table, the column of “Amount (parts)” indicates the content of the resin or the total content of components other than the resin (all units are “parts by mass”) in a case where the total mass of the composition is set to 100 parts by mass.

The “Present” in the column of “pH adjusting agent” means that, as described above, any one of citric acid or monoethanolamine was used to adjust the pH of the composition.

The column of “Mixed liquid” indicates the number of the mixed liquid used for preparing the composition. In addition, “Remainder” in the column of “Mixed liquid” means that the residue other than the resin E-1 and the pH adjusting agent in the composition is a mixed liquid shown in the column of “Mixed liquid”.

The column of “pH” indicates the pH of the composition at 25° C.

The numerical values shown in each column of Table 4 mean the content (unit: part by mass) of each component having a numerical value in a case where the total mass of each mixture is set to 100 parts by mass.

TABLE 1

| | | | Exam- | Exam- | Exam- | Exam- | Exam- | Exam- | Exam- | Exam- |
|---|---|---|---|---|---|---|---|---|---|---|
| | | | ple A1 | ple A2 | ple A3 | ple A4 | ple A5 | ple A6 | ple A7 | ple A8 |
| Composition | Resin | Kind | E-1 | E-2 | E-3 | E-4 | E-5 | E-6 | E-7 | E-8 |
| | | ClogP | 1.8 | 2.8 | 2.7 | 1.4 | 1.3 | 0.7 | 2.9 | 1.8 |
| | | Ratio a/b | 30/70 | 50/50 | 10/90 | 50/50 | 50/50 | 80/20 | — | 50/50 |
| | | Amount (parts) | 0.05 | 0.05 | 0.05 | 0.05 | 0.05 | 0.05 | 0.05 | 0.05 |
| | | Kind | | | | | | | | |
| | | ClogP | | | | | | | | |
| | | Ratio a/b | | | | | | | | |
| | | Amount (parts) | | | | | | | | |
| | pH adjusting | Citric acid | 1.0 | 1.0 | 1.0 | 1.0 | 1.0 | 1.0 | 1.0 | 1.0 |
| | agent | Monoethanolamine | 4.0 | 4.0 | 4.0 | 4.0 | 4.0 | 4.0 | 4.0 | 4.0 |
| | Mixed liquid | | C-1 | C-1 | C-1 | C-1 | C-1 | C-1 | C-1 | C-1 |
| | | | 94.95 | 94.95 | 94.95 | 94.95 | 94.95 | 94.95 | 94.95 | 94.95 |
| | pH | | 8.8 | 9.0 | 9.0 | 9.0 | 9.0 | 9.0 | 9.0 | 9.0 |
| Evaluation | Solubility | W | A | C | B | B | B | B | A | A |
| | | TiAlC | A | C | B | A | A | B | A | A |

| | | | | | | | | |
|---|---|---|---|---|---|---|---|---|
| Residue removability | A | A | A | A | A | A | A | A |
| Residual properties after rinsing | A | A | A | A | A | A | C | B |

TABLE 2

| | | | Exam- | Exam- | Exam- | Exam- | Exam- | Exam- | Exam- | Exam- |
|---|---|---|---|---|---|---|---|---|---|---|
| | | | ple A9 | ple A10 | ple A11 | ple A12 | ple A13 | ple A14 | ple A15 | ple A16 |
| Composition | Resin | Kind | E-9 | E-10 | E-11 | E-12 | E-13 | E-4 | E-15 | E-16 |
| | | ClogP | 1.8 | 1.8 | 1.8 | 1.8 | 2.5 | 1.4 | 1.0 | 0.7 |
| | | Ratio a/b | 5/95 | 90/10 | 30/70 | 30/70 | 50/50 | 50/50 | 50/50 | 40/60 |
| | | Amount (parts) | 0.05 | 0.05 | 0.05 | 0.05 | 0.20 | 0.03 | 0.08 | 0.01 |
| | | Kind | | | | | | E-14 | | |
| | | ClogP | | | | | | 0.8 | | |
| | | Ratio a/b | | | | | | 50/50 | | |
| | | Amount (parts) | | | | | | 0.03 | | |
| | pH adjusting | Citric acid | 1.0 | 1.0 | 1.0 | 0.5 | 1.0 | 1.2 | 1.0 | 1.0 |
| | agent | Monoethanolamine | 4.0 | 4.0 | 4.0 | 4.0 | 4.0 | 4.0 | 4.0 | 3.5 |
| | Mixed liquid | | C-1 | C-1 | C-1 | C-1 | C-5 | C-3 | C-4 | C-2 |
| | | | 94.95 | 94.95 | 94.95 | 95.45 | 94.80 | 94.77 | 94.92 | 95.49 |
| | pH | | 9.0 | 9.0 | 9.0 | 9.0 | 2.0 | 10.0 | 3.0 | 12.0 |
| Evaluation | Solubility | W | B | A | B | A | A | A | A | A |
| | | TiAlC | B | A | A | A | A | A | A | A |

| | | | | | | | | |
|---|---|---|---|---|---|---|---|---|
| Residue removability | A | A | A | A | B | A | A | B |
| Residual properties after rinsing | A | B | A | B | A | A | A | A |

TABLE 3

| | | | | | | | | | | | | Compar- |
|---|---|---|---|---|---|---|---|---|---|---|---|---|
| | | | | | | | | | | | | ative |
| | | | Exam- | Exam- | Exam- | Exam- | Exam- | Exam- | Exam- | Exam- | Exam- | Exam- |
| | | | ple A17 | ple A18 | ple A19 | ple A20 | ple A21 | ple A22 | ple A23 | ple A24 | ple A25 | ple A1 |
| Composition | Resin | Kind | E-1 | E-17 | E-18 | E-19 | E-20 | E-21 | E-22 | E-23 | E-24 | CE-1 |
| | | ClogP | 1.8 | 0.8 | 0.7 | 0.7 | 1.8 | 0.9 | 1.9 | 1.8 | 1.9 | 0.4 |
| | | Ratio a/b | 30/70 | 70/30 | 10/90 | 10/90 | 10/90 | 10/90 | 10/90 | 30/70 | 10/90 | — |
| | | Amount (parts) | 0.05 | 0.08 | 0.05 | 0.05 | 0.05 | 0.05 | 1.05 | 0.05 | 1.05 | 0.05 |
| | | Kind | | | | | | | | | | |
| | | ClogP | | | | | | | | | | |
| | | Ratio a/b | | | | | | | | | | |
| | | Amount (parts) | | | | | | | | | | |
| | pH adjusting | Citric acid | 1.0 | 1.0 | 1.0 | 1.0 | 1.0 | 1.0 | 1.0 | 1.0 | 1.0 | 1.0 |
| | agent | Monoethanolamine | 4.0 | 4.0 | 4.0 | 4.0 | 4.0 | 4.0 | 4.0 | 4.0 | 4.0 | 4.0 |
| | Mixed liquid | | C-6 | C-1 | C-1 | C-1 | C-3 | C-1 | C-2 | C-1 | C-2 | C-1 |
| | | | 94.95 | 94.92 | 94.95 | 94.95 | 94.95 | 94.95 | 93.95 | 94.95 | 93.95 | 94.95 |
| | pH | | 7.0 | 8.0 | 8.8 | 9.0 | 9.0 | 9.0 | 10.0 | 8.8 | 10.0 | 9.0 |
| Evaluation | Solubility | W | A | A | A | A | A | A | A | A | A | D |
| | | TiAlC | A | A | A | A | A | A | A | A | A | D |

| | | | | | | | | | | |
|---|---|---|---|---|---|---|---|---|---|---|
| Residue removability | B | A | A | A | A | A | A | A | A | B |
| Residual properties after rinsing | A | A | A | A | A | A | A | A | A | C |

TABLE 4

| Mixed liquid | | C-1 | C-2 | C-3 | C-4 | C-5 | C-6 |
|---|---|---|---|---|---|---|---|
| Removing | Hydroxylamine | 5.0 | | 4.5 | | | |
| agent | Ammonium hydroxide | | 5.0 | | | | |
| | Tetramethylammonium hydroxide | | | 0.5 | | | |
| | Hydrofluoric acid | | | | 5.0 | | |
| | Sulfuric acid | | | | | 2.5 | |
| Oxidizing | Hydrogen peroxide | | | | | 2.5 | |
| agent | | | | | | | |
| Corrosion | 5-methyl-1H-benzotriazole | 0.06 | 0.06 | | | 0.06 | |
| inhibitor | 5-mercapto-1-phenyl-1H-tetrazole | | | 0.06 | | | |
| | n-dodecyl mercaptan | | | | 0.06 | | |
| Surfactant | PERSOFT SF-T | 1.0 | | | | | |
| | NISSAN CATION BB | | 1.0 | | | | |
| | NONION K-220 | | | | 1.0 | | |
| | NISSAN ANON BL | | | | | 1.0 | |
| Antifoaming | KF-6701 | | 1.0 | | | | |
| agent | | | | | | | |
| Solvent | Propylene glycol | | | 4.0 | | | |
| | Water | 93.94 | 92.94 | 90.94 | 93.94 | 93.94 | 100 |

| | | | | | | |
|---|---|---|---|---|---|---|
| Total (parts by mass) | 100 | 100 | 100 | 100 | 100 | 100 |

As it is apparent from the results in Tables 1 to 3, it was confirmed that the compositions of Examples A1 to A25 according to the present invention have an excellent solubility for the W film, an excellent solubility for the TiAlC film, an excellent removal performance for etching residues, and excellent residual properties of the composition after the rinsing treatment. On the other hand, the composition of Comparative Example A1 which contained the nitrogen-containing resin different from the specific resin had low evaluations in terms of the solubility for the W film and the solubility for the TiAlC film, and thus the above-described effect was not obtained.

In addition, from the results in Tables 1 to 3, it was confirmed that in a case where the specific resin has the repeating unit A derived from a polymerizable compound having at least one selected from the group consisting of a primary amino group, a secondary amino group, a tertiary amino group, and salts thereof, the performance of suppressing the dissolution of the W film and the TiAlC film is more excellent (the comparison between Example A2 and Examples A3 to A5), and it was confirmed that in a case where the specific resin has the repeating unit A derived from a polymerizable compound having a primary amino group or a salt thereof, the performance of suppressing the dissolution of the W film is still more excellent (the comparison between Examples A2 to A5 and Examples A6 to A8 and the like).

It was confirmed that in a case where the specific resin has the repeating unit A derived from a polymerizable compound having an aromatic ring, the performance of suppressing the dissolution of the W film and the Ti alloy metal film is more excellent (the comparison between Example A6 and Example A1, A8, and the like).

It was confirmed that in a case where the specific resin has the repeating unit B and the ratio a/b is 10/90 or more, the effect of the present invention is more excellent (the comparison between Example A9 and Example A1 and the like).

It was confirmed that in a case where the specific resin has the repeating unit B and the ratio a/b is 80/20 or less, the residual properties of the composition after rinsing are more excellent (the comparison between Examples A7 and A10 and Examples A6 and the like).

It was confirmed that in a case where the specific resin has the repeating unit B having a carboxylic acid group, the residual properties of the composition after rinsing are more excellent (the comparison between Example A8 and Example A1 and the like).

It was confirmed that in a case where the weight-average molecular weight Mw of the specific resin is 1,000 or more, the effect of the present invention is more excellent (the comparison between Example A11 and Example A1 and the like).

It was confirmed that in a case where the weight-average molecular weight Mw of the specific resin is 500,000 or less, the residual properties of the composition after rinsing are more excellent (the comparison between Example A12 and Example A14 and the like).

Further, the dissolution rate, the residue removability, and the residual rate after rinsing were evaluated for the compositions of Examples A1 to A25 according to the above-described method, except that instead of the W film, a specimen obtained by laminating, on a substrate, a film consisting of a W-based metal material obtained by adding, to tungsten, one element selected from the group consisting of boron, carbon, nitrogen, and phosphorus was produced. As a result, it was confirmed that regarding the compositions of Example A1 to Example A25, the dissolution rate, the residue removability, and the residual rate after rinsing with respect to the above-described film consisting of a W-based metal material are also as excellent as the case of being applied to the W film.

### Example B

### [Preparation of Composition]

A preparation method for each of the compositions of Examples B1 to B9 and Comparative Examples B1 and B2 will be described with reference to Example B1.

The resin E-1, citric acid, trishydroxymethylaminomethane (Tris), and ultrapure water were mixed such that amounts thereof were the contents shown in Table 5 described below. Then potassium hydroxide or nitric acid as a pH adjusting agent was added thereto such that the pH of the composition to be prepared was 6.0, and the obtained mixed liquid was sufficiently stirred with a stirrer to prepare a composition of Example B1.

According to the preparation method for the composition of Example B1, each of compositions of Examples B2 to B9 and Comparative Examples B1 and B2 having the compositions shown in Table 5 was prepared.

### [Evaluation of Composition]

### <Residue Removability>

Using each of the prepared compositions, the residue removability (washing performance) was evaluated in a case where a metal film subjected to the CMP treatment was washed.

Using FREX-300SII (a polishing device, manufactured by Ebara Corporation), a wafer (diameter: 12 inches) having a metal film consisting of tungsten on a surface was subjected to a CMP treatment using a polishing liquid (W2000, manufactured by Cabot Corporation) under conditions of a polishing liquid supply rate of 0.28 mL/(min cm²), a polishing pressure of 2.0 psi, and a polishing time of 60 seconds.

Thereafter, the temperature of each composition was adjusted to room temperature (23° C.), and scrub washing was carried out using each composition for 60 seconds, followed by drying. The number of defects on the polished surface of the obtained wafer was detected using a defect detection device, and each defect was observed with a scanning electron microscope (SEM) and subjected to defect classification. In a case of being necessary, the constitutional elements were analyzed by energy dispersion type X-ray analysis apparatus (EDAX) to specify the components. In this manner, the number of defects based on the residues due to the CMP treatment (the number of target defects) was determined, and the washing performance was evaluated according to the following evaluation standard (an evaluation 6 indicates that the washing performance is most excellent).

### (Evaluation Standard for Residue Removability)

6: The number of target defects is less than 20.

5: The number of target defects is 20 or more and less than 50.

4: The number of target defects is 50 or more and less than 100.

3: The number of target defects is 100 or more and less than 200.

2: The number of target defects is 200 or more and less than 300.

1: The number of target defects is 300 or more.

### <Solubility>

Using each composition, the solubility for the metal film was evaluated in a case where the metal film was washed.

A wafer (diameter: 12 inches) having a W film consisting of a tungsten simple body (W) on the surface was cut to prepare a 2 cm×2 cm square wafer coupon. The thickness of the W film was set to 200 nm. The wafer coupon was immersed in each composition (liquid temperature: 23° C.) for 30 minutes under a stirring condition of a stirring rotation speed of 250 rpm.

Before and after the immersion test, the film thickness of the W film was measured with an optical film thickness meter Ellipsometer M-2000 (manufactured by J. A. Woollam Co.). From the measured film thicknesses before and after immersion, the dissolution rate (A/min) of the W film in a case where each composition was used was calculated. From the calculated dissolution rate, the solubility of the W film (the performance of suppressing the dissolution of the W film) was evaluated based on the following evaluation standard. It is indicated that the slower the dissolution rate is, the more suppression of the dissolution of the W film occurs by the composition (an evaluation 6 indicates that the solubility is most excellent).

### (Evaluation Standard for Solubility of W Film)

6: The dissolution rate of the W film is 0.2 Å/min or less.

5: The dissolution rate of the W film is 0.2 Å/min or more and 0.5 Å/min or less.

4: The dissolution rate of the W film is 0.5 Å/min or more and 1 Å/min or less.

3: The dissolution rate of the W film is 1 Å/min or more and 3 Å/min or less.

2: The dissolution rate of the W film is 3 Å/min or more and 5 Å/min or less.

1: The dissolution rate of the W film is more than 5 Å/min.

Table 5 shows the composition of each of the compositions used in Examples B1 to B9 and Comparative Examples B1 and B2, and the evaluation results thereof.

In Table 5, the column of “ClogP” of “Resin” indicates a ClogP value of a polymerizable compound from which the repeating unit A (the repeating unit containing a nitrogen atom in the resin CE-1) contained in each resin is derived. The ClogP value was calculated according to the above-described method.

In Tables 1 to 3, the column of “Amount (%)” of each component indicates the content (unit: % by mass) of each component with respect to the total mass of the composition.

“Remainder” in the column of “Water” indicates that the water content is the remainder of the composition other than the resin, the removing agent, and the pH adjusting agent.

The numerical value in the column of “pH” indicates the pH of the above-described composition at 25° C. which is measured with the pH meter.

In the column of “Removing agent” in the table, “Tris” means trishydroxymethylaminomethane, “TMAH” means tetramethylammonium hydroxide, “MEA” means monoethanolamine, “Removing agent 1” means tris(2-hydroxyethyl)methylammonium hydroxide, and “Removing agent 2” means 2-(dimethylamino)-2-methyl-1-propanol.

TABLE 5

| | Composition | | | | | | | | | | |
|---|---|---|---|---|---|---|---|---|---|---|---|
| | Resin | | Removing agent | | | | | pH | | Evaluation | |
| | | Amount | | Amount | | Amount | | adjusting | | Residue | W film |
| | Kind | (%) | Kind | (%) | Kind | (%) | Water | agent | pH | removability | solubility |
| Example B1 | E-1 | 0.05 | Citric acid | 1.5 | Tris | 4.5 | Remainder | KOH, | 6.0 | 6 | 6 |
| | | | | | | | | HNO3 | | | |
| Example B2 | E-3 | 0.05 | Citric acid | 1.5 | Tris | 4.5 | Remainder | KOH, | 6.0 | 6 | 5 |
| | | | | | | | | HNO3 | | | |
| Example B3 | E-2 | 0.05 | Citric acid | 1.5 | Tris | 4.5 | Remainder | KOH, | 6.0 | 6 | 4 |
| | | | | | | | | HNO3 | | | |
| Example B4 | E-5 | 0.05 | Citric acid | 1.5 | Tris | 4.5 | Remainder | KOH, | 6.0 | 6 | 5 |
| | | | | | | | | HNO3 | | | |
| Example B5 | E-16 | 0.05 | Citric acid | 1.5 | Tris | 4.5 | Remainder | KOH, | 6.0 | 6 | 6 |
| | | | | | | | | HNO3 | | | |
| Example B6 | E-2 | 0.05 | Tartaric acid | 1 | TMAH | 2 | Remainder | KOH, | 5.0 | 6 | 5 |
| | | | | | | | | HNO3 | | | |
| Example B7 | E-3 | 0.05 | Succinic | 2 | MEA | 3 | Remainder | KOH, | 6.5 | 6 | 4 |
| | | | acid | | | | | HNO3 | | | |
| Example B8 | E-4 | 0.05 | Citric acid | 1 | Removing | 3.4 | Remainder | KOH, | 9.0 | 6 | 5 |
| | | | | | agent 1 | | | HNO3 | | | |
| Example B9 | E-5 | 0.05 | EDTA | 3 | Removing | 4.2 | Remainder | KOH, | 4.0 | 6 | 6 |
| | | | | | agent 2 | | | HNO3 | | | |
| Comparative | — | — | Citric acid | 1.5 | Tris | 4.5 | Remainder | KOH, | 6.0 | 6 | 2 |
| Example B1 | | | | | | | | HNO3 | | | |
| Comparative | CE-1 | 0.05 | Citric acid | 1.5 | Tris | 4.5 | Remainder | KOH, | 6.0 | 6 | 2 |
| Example B2 | | | | | | | | HNO3 | | | |

As shown in the table, it was confirmed that in the compositions of Examples B1 to B9 according to the present invention, both the solubility for the W film and the removal performance of residues on the wafer subjected to the CMP treatment are excellent. On the other hand, the compositions of Comparative Examples B1 and B2, which did not contain the specific resin, had a low evaluation of the solubility for the W film, and thus the above-described effect was not obtained.

In the evaluation test of the residue removal performance, a wafer having a metal film consisting of W on a surface was subjected to a CMP treatment, and then the polished surface of the wafer subjected to the CMP treatment was subjected to a buff polishing treatment.

In the buff polishing treatment, each composition adjusted to room temperature (23° C.) was used as a washing solution for buff polishing. In addition, a buff polishing treatment was carried out by using the polishing device used in the CMP treatment under conditions of a polishing pressure of 2.0 psi, a supply rate of the washing solution for buff polishing of 0.28 mL/(min·cm²), and a polishing time of 60 seconds.

Then, the wafer that had been subjected to the buff polishing treatment was washed over 30 seconds using each composition adjusted to room temperature (23° C.) and then subjected to a drying treatment.

As a result of evaluating the residue removal performance of the composition with respect to the obtained polished surface of the wafer according to the test method described in <Residue removability> described above, it was confirmed that the same evaluation results as those of the composition of each of Examples described above are exhibited.

### Explanation of References

1: substrate
2: metal layer
3: etching stop layer
4: insulating film
5: metal hard mask
6: hole
10: laminate
11: inner wall
11a: cross-sectional wall
11b: bottom wall
12: dry etching residues

What is claimed is:

1. A composition for a semiconductor device, comprising:
a resin having a repeating unit A derived from a polymerizable compound containing a nitrogen atom; and
water,
wherein a ClogP of the polymerizable compound is 0.5 or more, and
a solubility of the resin in water at 25° C. is 0.01% by mass or more.

2. The composition according to claim 1,
wherein the polymerizable compound includes at least one selected from the group consisting of compounds represented by Formulae (1) to (3),
CH₂=CR¹-L¹-(X′)ₙ₁ (1)
in Formula (1), R¹ represents a hydrogen atom or an alkyl group having 1 to 4 carbon atoms, L¹ represents an (n1+1)-valent linking group, X¹ represents a group selected from the group consisting of a primary amino group, a secondary amino group, a tertiary amino group, and salts thereof, as well as a quaternary ammonium cationic group, and n1 represents an integer of 1 to 5, where in a case where n1 is an integer of 2 to 5, a plurality of X” 's may be the same or different from each other,
CH₂=CR²—X² (2)
in Formula (2), R² represents a hydrogen atom or an alkyl group having 1 to 4 carbon atoms, and X² represents a nitrogen-containing heterocyclic ring having at least one group selected from the group consisting of a primary amino group, a secondary amino group, a tertiary amino group, and salts these groups, and a quaternary ammonium cationic group, 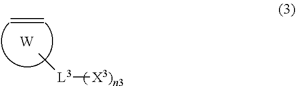
in Formula (3), a ring W represents a hydrocarbon ring or a heterocyclic ring, which has an ethylenically unsaturated group, L³ represents a single bond or an (n3+1)-valent linking group, X³ represents a group selected from the group consisting of a primary amino group, a secondary amino group, a tertiary amino group, and salts these groups, and a quaternary ammonium cationic group, and n3 represents an integer of 1 to 5, where in a case where n3 is an integer of 2 to 5, a plurality of X³'s may be the same or different from each other.

3. The composition according to claim 1,
wherein the polymerizable compound has at least one selected from the group consisting of a primary amino group, a secondary amino group, a tertiary amino group, and salts thereof.

4. The composition according to claim 1,
wherein the polymerizable compound has a primary amino group or a salt thereof.

5. The composition according to claim 1,
wherein the polymerizable compound has an aromatic ring.

6. The composition according to claim 1,
wherein the polymerizable compound is a compound represented by Formula (1a), 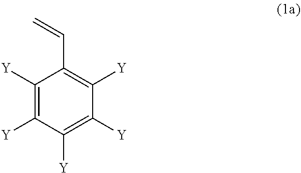
in the formula, Y's each independently represent a hydrogen atom or -L²-L³-NH₂, provided that at least two Y's represent -L²-L³-NH₂, L² represents a single bond or an oxygen atom, and L³ represents an aliphatic hydrocarbon group having 1 to 5 carbon atoms.

7. The composition according to claim 1,
wherein the resin further has a repeating unit B having an acid group.

8. The composition according to claim 7,
wherein the acid group is a carboxy group.

9. The composition according to claim 7,
wherein a ratio a/b of a molar number a of the repeating unit A to a molar number b of the repeating unit B is 10/90 to 80/20 where the repeating unit A and the repeating unit B are contained in the resin.

10. The composition according to claim 1,
wherein the resin has a weight-average molecular weight of 1,000 to 500,000.

11. The composition according to claim 1, further comprising:
a removing agent.

12. The composition according to claim 1,
wherein a content of the resin is 10 to 10,000 ppm by mass with respect to a total mass of the composition.

13. The composition according to claim 1, further comprising:
at least one selected from the group consisting of an oxidizing agent, a corrosion inhibitor, a surfactant, an antifoaming agent, and an organic solvent.

14. The composition according to claim 1,
wherein the composition is a composition for a substrate having a metal-containing substance which contains at least one of tungsten or a tungsten alloy.

15. The composition according to claim 1,
wherein the composition is used as a washing solution for removing etching residues or a washing solution for removing residues from a substrate after chemical mechanical polishing.

16. A substrate treatment method comprising:
a step A of removing a metal-containing substance on a substrate by using the composition according to claim 1.

17. The substrate treatment method according to claim 16, further comprising:
a step C of subjecting, after the step A, the substrate obtained in the step A to a rinsing treatment by using a rinsing liquid.

18. A manufacturing method for a semiconductor device, comprising:
a step of treating a substrate by using the composition according to claim 1.

19. A compound represented by Formula (1a), 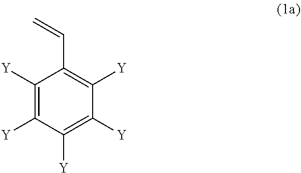
in the formula, Y's each independently represent a hydrogen atom or -L²-L³-NH₂, provided that at least two Y's represent -L²-L³-NH₂, L² represents a single bond or an oxygen atom, and L³ represents an aliphatic hydrocarbon group having 1 to 5 carbon atoms.

20. A resin comprising:
a repeating unit A derived from the compound according to claim 19.

21. A composition comprising:
the compound according to claim 19.

22. A compound represented by Formula (1b), 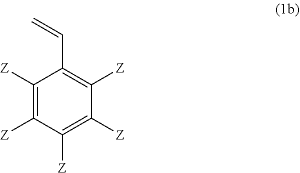
in the formula, Z's each independently represent a hydrogen atom or -L²-L³-OH, provided that at least two Z's represent -L²-L³-OH, L² represents a single bond or an oxygen atom, and L³ represents an aliphatic hydrocarbon group having 1 to 5 carbon atoms.
